(19) United States
(12) Patent Application Publication
(10) Pub. No.: US 2024/0190823 A1
(43) Pub. Date: Jun. 13, 2024

(54) NRF2 INHIBITORS AND USES THEREOF

(71) Applicant: Board of Trustees of Michigan State University, East Lansing, MI (US)

(72) Inventors: Karen Liby, Holt, MI (US); Di Zhang, Okemos, MI (US); Aaron Odom, East Lansing, MI (US); Zhilin Hou, East Lansing, MI (US)

(21) Appl. No.: 18/548,058

(22) PCT Filed: Feb. 25, 2022
(86) PCT No.: PCT/US2022/017971

§ 371 Date: Aug. 25, 2023

Related U.S. Application Data
(60) Provisional application No. 63/154,306, filed on Feb. 26, 2021.

(51) Int. Cl. C07D 213/74; A61P 35/00; C07D 213/85; C07D 215/58; C07D 241/26; C07D 401/04; C07D 401/12; C07D 471/04

(52) U.S. Cl. CPC C07D 213/74; A61P 35/00; C07D 213/85; C07D 215/58; C07D 241/26; C07D 401/04; C07D 401/12; C07D 471/04

(57) ABSTRACT

The disclosure relates to compounds of the formulae (I), (II), (lla), (III), (IV), and (IVa) and pharmaceutically acceptable salts, polymorphs, prodrugs, solvates or clathrates thereof, and pharmaceutical compositions comprising such compounds. The disclosure also relates to treating cancer with such compounds and compositions.

### CROSS-REFERENCE TO RELATED APPLICATIONS

This application claims the benefit of U.S. Provisional Appl. Ser. No. 63/154,306, filed Feb. 26, 2021, which is incorporated by reference as if fully set forth herein.

### BACKGROUND

As a master regulator of homeostasis, the Nuclear factor erythroid-2-related factor 2-Kelch-like ECH-associated protein 1-ARE (Nrf2-Keap1-ARE) pathway plays critical roles in various cellular processes including redox-balancing, detoxification, proliferation, inflammation, and metabolism. Nuclear factor erythroid-2-related factor 2 (Nrf2) belongs to a subfamily of basic leucine zipper (bZIP) proteins. As a transcription factor, the activity of Nrf2 is tightly controlled. Under basal conditions, Kelch-like ECH-associated protein 1 (Keap1), acting as an adaptor protein, binds to Nrf2 and targets it to the Cullin3-based ubiquitin E3 ligase complex for protein degradation. Upon exposure to cellular stresses, Nrf2 is released from Keap1 and translocates into the nucleus to activate the expression of a network of downstream cytoprotective genes.

Nrf2 signaling protects cells against both endogenous and exogenous insults. Carcinogenesis is exacerbated in Nrf2-knockout mice vs. wildtype mice in a wide variety of preclinical models, including skin cancer induced by ultraviolet light, liver cancer induced by aflatoxin, cancer in the forestomach induced by polycyclic hydrocarbons, bladder cancer induced by nitrosamines, and colon cancer induced by inflammation. Numerous Nrf2 activators have been tested as chemopreventive agents to prevent or delay tumor development, and the beneficial effects of these agents are greatly dampened in Nrf2 KO mice.

However, recently loss-of-function mutations in KEAP1 and gain-of-function mutations in NFE2L2 (gene encoding Nrf2) have been discovered in many human cancers, especially lung cancers. Up to 30% of human lung cancers were found to have either KEAP1 (deletions and mutations, predominately in lung adenocarcinomas) or NFE2L2 (amplifications and mutations, predominately in lung squamous cell carcinomas) genetic alterations. These genetic changes in the tumors provide a growth-promoting role for this pathway. Accumulating evidence has demonstrated that constitutive activation of the Nrf2 pathway, via genetic or epigenetic mechanisms, favors tumor growth via several mechanisms. Nrf2 acts downstream of key oncoproteins including Kras, Myc, BRAF and PI3K to promote tumor cell survival and growth. Nrf2 can also regulate cancer metabolism by reprograming cells into anabolic pathways for rapid biosynthesis. The expression of various metabolic enzymes and transporters is directly regulated via Nrf2. Moreover, the constitutive activation of Nrf2 is associated with chemoresistance, and inhibiting Nrf2 expression sensitizes tumor cells to chemotherapies.

In contrast to Nrf2 activators, only a few Nrf2 inhibitors have been reported. Brusatol was the first Nrf2 inhibitor identified, and it remains the most potent known inhibitor. However, a mass spectrometry profiling study suggested that brusatol functions as a global protein synthesis inhibitor rather than a specific inhibitor of Nrf2. High throughput screens also identified AEM1, ML385, and IM3829 as small molecules that inhibit Nrf2 activity. These molecules possess different chemical properties and kinetics to inhibit Nrf2, but all significantly enhanced the susceptibility of Keap1 mutant tumors to radiotherapy or chemotherapies. However, none of them has been widely used because of either lack of efficacy or selectivity.

### SUMMARY

There is therefore a need for efficacious and/or selective small molecules that inhibit Nrf2 that do not have the drawbacks of known compounds described herein.

To develop Nrf2 inhibitors, the inventors screened and identified a series of small molecules that were shown to inhibit the Nrf2 pathway both in vitro and in vivo. In vitro, the expression of Nrf2 and its downstream targets, redox-balancing, and cell proliferation were measured following treatment with the compounds described herein. In vivo, the efficacy of the compounds described herein, alone or in combination with chemotherapy, e.g., carboplatin, for treating KEAP1 mutant tumors in a model of non-small cell lung cancer (NSCLC) were also evaluated.

### BRIEF DESCRIPTION OF THE DRAWINGS

FIG. 1A is the chemical structure of compound 1.

FIG. 1B is a plot of inhibition of Nrf2 transcriptional activity (measured using a luciferase assay) as a function of the concentration of compound 1.

FIG. 1C a plot of cell viability as a function of the concentration of compound 1.

FIG. 1D is a plot of change in gene expression of the noted genes following treatment with compound 1.

FIG. 1E is a photograph of a western blot following treatment of A549 cells with compound 1 for 24 hours

FIG. 2A is a photograph of a western blot following treatment of A549 cells with compound 1 for 1-24 hours.

FIG. 2B is a is a plot of change in gene expression as a function of time following treatment with compound 1.

FIGS. 2C-2F are photographs of western blots following treatment with compound 1 under various conditions.

FIGS. 3A-E are photographs of western blots following treatment with compound 1 under various conditions.

FIG. 4A is a plot of reactive oxygen species as a function of compound 1 concentration, where cells have been stimulated with tert-butyl hydroperoxide (tBHP) for 15 minutes.

FIG. 4B is a plot of percent inhibition of cell proliferation as a function of the log of the concentration of compound 1. The compounds described herein generally exhibit a percent inhibition of Nrf2 expression at 5 μM of at least 10%, at least 15%, at least 20%, at least 25%, at least 30%, at least 35%, at least 40%, at least 45%, at least 50%, at least 55%, at least 60% or higher inhibition of Nrf2 expression, such as from about 10% to about 60%, about 15% to about 70%, about 20% to about 60%, about 25% to about 75%, about 30% to about 80%, about 35% to about 75%,a bout 50% to about 80%, about 40% to about 90%, about 45% to about 65%, about 50% to about 95%, about 50% to about 65%, about 75% to about 99% or about 50% to about 85% Nrf2 expression at a 5 μM concentration of the compounds described herein. Compound 1, for example, exhibits a 51.04±11.35% Nrf2 inhibition at a 5 μM concentration of the compound.

FIG. 4C is photographs of A549, H460 and A427 cells that were plated in 0.6% soft agar for 7 days and treated with increasing concentrations of compound 1.

FIG. 5A is grids showing percent inhibition of cell proliferation as a function of treatment with compound 1 and various chemotherapies in vitro.

FIG. 5B is a plot of tumor volume as a function of various treatments, including treatment with compound 1.

FIG. 5C is a plot of body weight as a function of various treatments, including treatment with compound 1.

FIG. 5D is photographs of the amount of proliferation (determined by PCNA immunohistochemical staining) in tumors 4 mm in diameter treated with either vehicle, carboplatin (5 mg/kg), compound 1 (50 mg/kg, BID) or the combination for 4 weeks.

FIG. 5E is photographs of Nrf2 expression in tumors as evaluated by immunohistochemistry.

It is to be understood that the drawings are not intended to limit the scope of the present teachings in any way.

### DESCRIPTION

The above and other objects, features, and advantages of the present invention will become more apparent when the above drawings are taken in conjunction with the following description.

The disclosure relates to a compound of the formula (I):

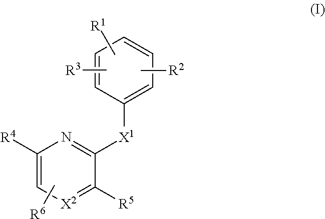

or a pharmaceutically acceptable salt, polymorph, prodrug, solvate or clathrate thereof, wherein:
R¹—R⁶ are each independently H, alkyl, aryl, arylalkyl, OR⁷, cyano, amino, amido or halo, wherein R⁷ is H, alkyl, arylalkyl or a protecting group or R⁴ and R⁶, together with the atoms to which they are attached, form an aryl or a heteroaryl group;
X¹ is X³, —O—X⁴—, or —N(R⁸)—X⁴—, wherein X³ is alkyl (e.g., CH₂), X⁴ is a bond or alkyl, and R⁸ is H, alkyl, arylalkyl or acyl; and
X² is CR¹ or N.

In compounds of formula (I), R⁶ can be alkyl (e.g., 1 to 6 carbon atoms (C₁-C₆), such as methyl, ethyl, n-propyl, n-butyl, n-pentyl, n-hexyl, n-heptyl, n-octyl, isopropyl, iso-butyl, sec-butyl, t-butyl, neopentyl, isopentyl, and 2,2-dimethylpropyl groups) or aryl (e.g., aryl groups contain from 6 to 10 carbon atoms (C₆-C₁₀) in the ring portions of the groups, such as phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups). Alternatively, or in combination, in the compounds of formula (I), X² can be N. Or alternatively, or in combination, in the compounds of formula (I), X² can be CR¹, wherein R¹ can be H. Alternatively, or in combination, in the compounds of formula (I), R¹—R³ can each independently H, alkyl (e.g., 1 to 6 carbon atoms (C₁-C₆), such as methyl, ethyl, n-propyl, n-butyl, n-pentyl, n-hexyl, n-heptyl, n-octyl, isopropyl, iso-butyl, sec-butyl, t-butyl, neopentyl, isopentyl, and 2,2-dimethylpropyl groups), OR⁷ (wherein R⁷ is H, alkyl, arylalkyl (such as benzyl) or a protecting group or halo, such as fluoro (F), bromo (Br) or chloro (CI)). Alternatively, or in combination, in the compounds of formula (I), R⁵ can be cyano (C≡N) or amido (e.g., —C(O)NR², wherein each R can be H, alkyl, arylalkyl or arylalkyl). Alternatively, or in combination, in the compounds of formula (I), R⁴ can be H or alkyl (e.g., 1 to 6 carbon atoms (C₁-C₆), such as methyl, ethyl, n-propyl, n-butyl, n-pentyl, n-hexyl, n-heptyl, n-octyl, isopropyl, iso-butyl, sec-butyl, t-butyl, neopentyl, isopentyl, and 2,2-dimethylpropyl groups). For example, in the compounds of the formula (I), R⁴ can be H. Alternatively, or in combination, in the compounds of formula (I), X¹ can be —N(R⁵)—X⁴—. For example, X⁴ can be a bond, such that the X¹ becomes a group of the formula —N(R⁵)—, such that the nitrogen atom bridges the ring comprising the groups R¹—R³ and the ring comprising R⁴—R⁶ as follows:

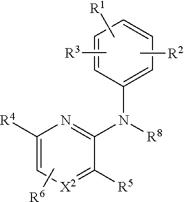

In the compounds of formula (I), R⁵ can be H, alkyl (e.g., 1 to 6 carbon atoms (C₁-C₆), such as methyl, ethyl, n-propyl, n-butyl, n-pentyl, n-hexyl, n-heptyl, n-octyl, isopropyl, iso-butyl, sec-butyl, t-butyl, neopentyl, isopentyl, and 2,2-dimethylpropyl groups) or acyl (e.g., —C(O)R, wherein R is H, alkyl, aryl or arylalkyl).

The disclosure also relates to compounds of the formula (I), wherein the compound of the formula (I) is a compound of the formula (II):

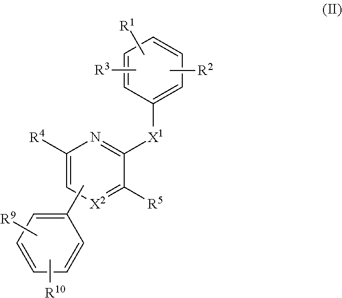

or a pharmaceutically acceptable salt, polymorph, prodrug, solvate or clathrate thereof, wherein:
R¹—R⁵, R⁹, and R¹⁹ are each independently H, alkyl, aryl, arylalkyl, OR⁷, cyano, amino, amido or halo, wherein R⁷ is H, alkyl, arylalkyl or a protecting group;
X¹ is X³, —O—X⁴—, or —N(R⁸)—X⁴—, wherein X³ is alkyl (e.g., CH₂), X⁴ is a bond or alkyl, and R⁸ is H, alkyl, arylalkyl or acyl; and
X² is CR¹ or N.

Compounds of the formula (II) include compounds of the formula (IIa):

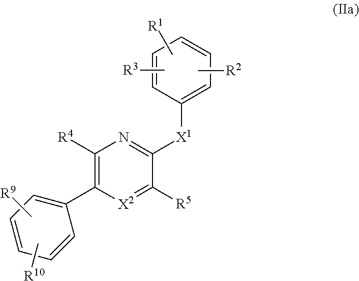

or a pharmaceutically acceptable salt, polymorph, prodrug, solvate or clathrate thereof.

In compounds of formula (II) or (IIa), X² can be N. Or alternatively, or in combination, in the compounds of formula (II) or (II), X² can be CR¹, wherein R¹ can be H. Alternatively, or in combination, in the compounds of formula (II) or (IIa), R¹—R³ can each independently H, alkyl (e.g., 1 to 6 carbon atoms (C₁-C₆), such as methyl, ethyl, n-propyl, n-butyl, n-pentyl, n-hexyl, n-heptyl, n-octyl, isopropyl, iso-butyl, sec-butyl, t-butyl, neopentyl, isopentyl, and 2,2-dimethylpropyl groups), OR⁷ (wherein R⁷ is H, alkyl, arylalkyl (such as benzyl) or a protecting group or halo, such as fluoro (F), bromo (Br) or chloro (Cl)). Alternatively, or in combination, in the compounds of formula (II) or (IIa), R⁵ can be cyano (C≡N) or amido (e.g., —C(O)NR₂, wherein each R can be H, alkyl, arylalkyl or arylalkyl). Alternatively, or in combination, in the compounds of formula (II) or (IIa), R⁴ can be H or alkyl (e.g., 1 to 6 carbon atoms (C₁-C₆), such as methyl, ethyl, n-propyl, n-butyl, n-pentyl, n-hexyl, n-heptyl, n-octyl, isopropyl, iso-butyl, sec-butyl, t-butyl, neopentyl, isopentyl, and 2,2-dimethylpropyl groups). For example, in the compounds of the formula (II) or (IIa), R⁴ can be H. Alternatively, or in combination, in the compounds of formula (II) or (IIa), X¹ can be —N(R⁸)—X⁴—. For example, X⁴ can be a bond, such that the X¹ becomes a group of the formula —N(R⁸)—, such that the nitrogen atom bridges the ring comprising the groups R¹—R³ and the ring comprising R⁴ and R⁵ as follows:

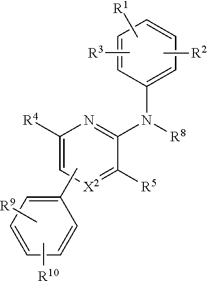

In the compounds of formula (II) or (IIa), R⁸ can be H, alkyl (e.g., 1 to 6 carbon atoms (C₁-C₆), such as methyl, ethyl, n-propyl, n-butyl, n-pentyl, n-hexyl, n-heptyl, n-octyl, isopropyl, iso-butyl, sec-butyl, t-butyl, neopentyl, isopentyl, and 2,2-dimethylpropyl groups) or acyl (e.g., —C(O)R, wherein R is H, alkyl, aryl or arylalkyl).

Also contemplated herein are compounds of the formula (IIb):

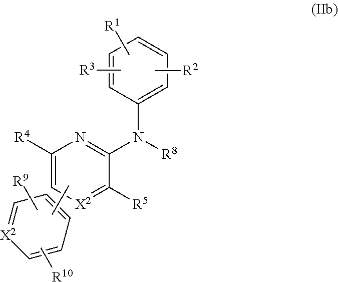

such as compounds of the formula:

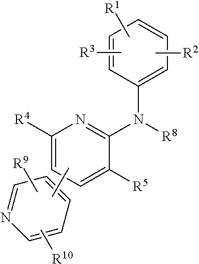

The disclosure also relates to compounds of the formula (I), wherein the compound of the formula (I) is a compound of the formula (IIc):

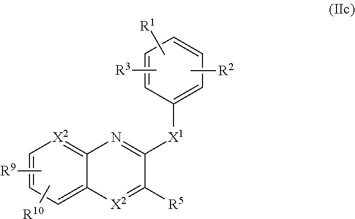

or a pharmaceutically acceptable salt, polymorph, prodrug, solvate or clathrate thereof, wherein:
R¹ —R³, R⁵, R⁹, and R¹⁹ are each independently H, alkyl, aryl, arylalkyl, OR⁷, cyano, amino, amido or halo, wherein R⁷ is H, alkyl, arylalkyl or a protecting group;
X¹ is X³, —O—X⁴—, or —N(R⁸)—X⁴—, wherein X³ is alkyl (e.g., CH 2), X⁴ is a bond or alkyl, and R⁸ is H, alkyl, arylalkyl or acyl; and
each X² is independently CR¹ or N.

Compounds of the formulae (I), (II), (IIa), (IIb), and (IIc) include, but are not limited to, compounds of the formulae:

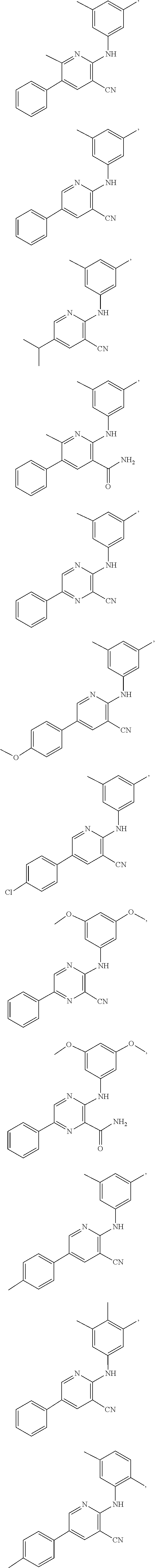

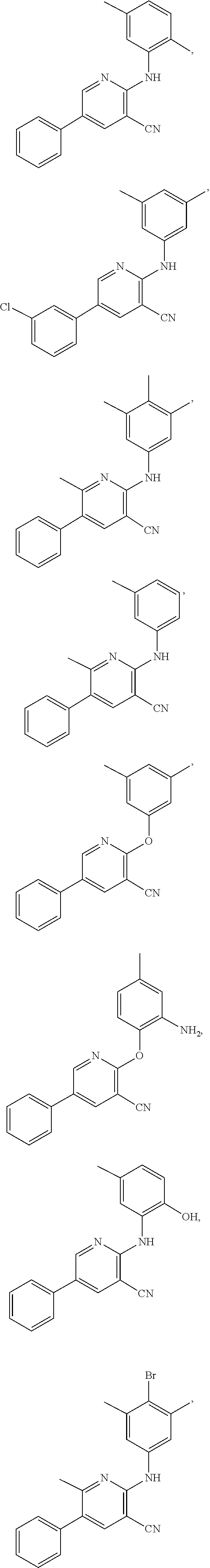

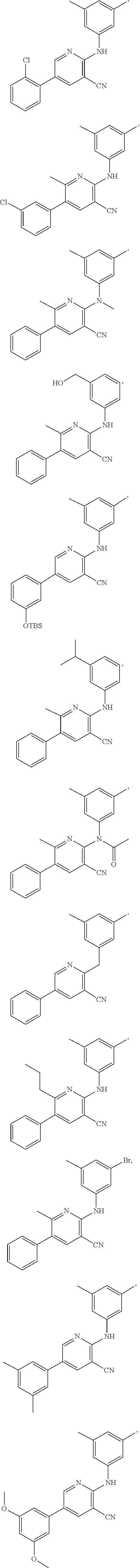

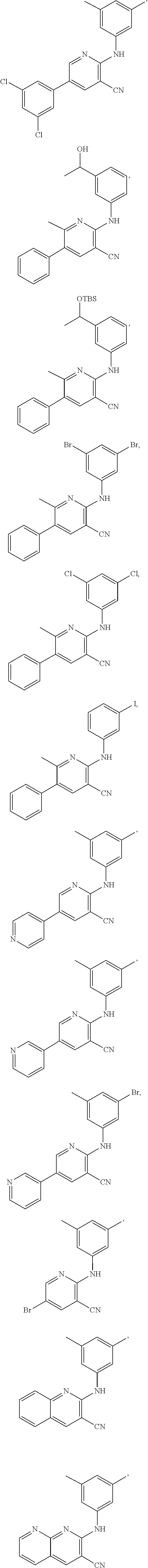

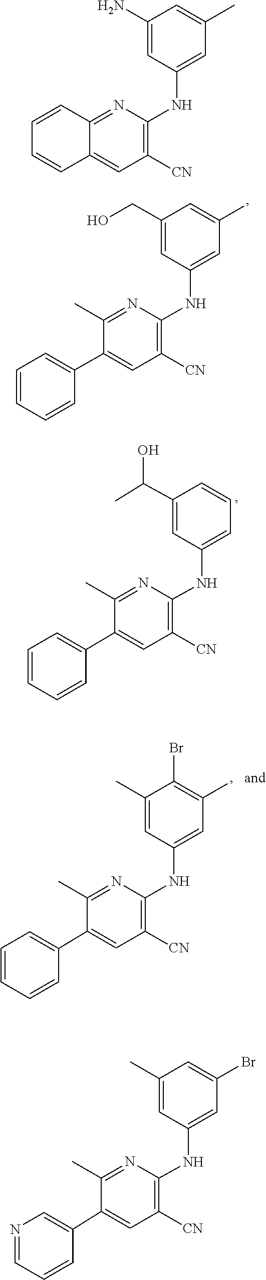

or a pharmaceutically acceptable salt, polymorph, prodrug, solvate or clathrate thereof.

The disclosure relates to a compound of the formula (III):

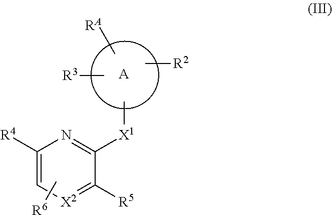

or a pharmaceutically acceptable salt, polymorph, prodrug, solvate or clathrate thereof, wherein:
A is aryl or heteroaryl;
R^{A} is H, alkyl, alkynyl, aryl, arylalkyl, OR⁷, C(O)OR⁷, cyano, amino, amido, or halo, wherein R⁷ is H, alkyl, arylalkyl or a protecting group;
R²—R⁶ are each independently H, alkyl, aryl, arylalkyl, OR⁷, cyano, amino, amido, or halo, wherein R⁷ is H, alkyl, arylalkyl or a protecting group;
X¹ is X³, —O—X⁴—, or —N(R⁸)—X⁴—, wherein X³ is alkyl (e.g., CH₂), X⁴ is a bond or alkyl, and R⁸ is H, alkyl, arylalkyl or acyl; and
X² is CR¹ or N.

In compounds of formula (III), R⁶ can be alkyl (e.g., 1 to 6 carbon atoms (C₁-C₆), such as methyl, ethyl, n-propyl, n-butyl, n-pentyl, n-hexyl, n-heptyl, n-octyl, isopropyl, iso-butyl, sec-butyl, t-butyl, neopentyl, isopentyl, and 2,2-dimethylpropyl groups) or aryl (e.g., aryl groups contain from 6 to 10 carbon atoms (C₆-C₁₀) in the ring portions of the groups, such as phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups). Alternatively, or in combination, in the compounds of formula (III), X² can be N. Or alternatively, or in combination, in the compounds of formula (III), X² can be CR¹, wherein R¹ can be H. Alternatively, or in combination, in the compounds of formula (III), R¹—R³ can each independently H, alkyl (e.g., 1 to 6 carbon atoms (C₁-C₆), such as methyl, ethyl, n-propyl, n-butyl, n-pentyl, n-hexyl, n-heptyl, n-5 octyl, isopropyl, iso-butyl, sec-butyl, t-butyl, neopentyl, isopentyl, and 2,2-dimethylpropyl groups), OR⁷ (wherein R⁷ is H, alkyl, arylalkyl (such as benzyl) or a protecting group or halo, such as fluoro (F), bromo (Br) or chloro (Cl)). Alternatively, or in combination, in the compounds of formula (III), R⁵ can be cyano (C≡N) or amido (e.g., —C(O)NR², wherein each R can be H, alkyl, arylalkyl or arylalkyl). Alternatively, or in combination, in the compounds of formula (III), R⁴ can be H or alkyl (e.g., 1 to 6 carbon atoms (C₁-C₆), such as methyl, ethyl, n-propyl, n-butyl, n-pentyl, n-hexyl, n-heptyl, n-octyl, isopropyl, iso-butyl, sec-butyl, t-butyl, neopentyl, isopentyl, and 2,2-dimethylpropyl groups). For example, in the compounds of the formula (III), R⁴ can be H. Alternatively, or in combination, in the compounds of formula (III), X¹ can be —N(R⁵)—X⁴—. For example, X⁴ can be a bond, such that the X¹ becomes a group of the formula —N(R⁵)—, such that the nitrogen atom bridges the ring comprising the groups R¹—R³ and the ring comprising R⁴—R⁶ as follows:

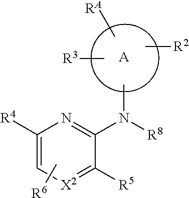

In the compounds of formula (III), R⁵ can be H, alkyl (e.g., 1 to 6 carbon atoms (C₁-C₆), such as methyl, ethyl, n-propyl, n-butyl, n-pentyl, n-hexyl, n-heptyl, n-octyl, isopropyl, iso-butyl, sec-butyl, t-butyl, neopentyl, isopentyl, and 2,2-dimethylpropyl groups) or acyl (e.g., —C(O)R, wherein R is H, alkyl, aryl or arylalkyl).

The disclosure also relates to compounds of the formula (III), wherein the compound of the formula (III) is a compound of the formula (IV):

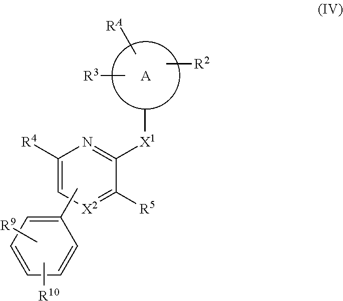

or a pharmaceutically acceptable salt, polymorph, prodrug, solvate or clathrate thereof, wherein:
R¹—R⁵, R⁹, and R¹⁹ are each independently H, alkyl, aryl, arylalkyl, OR⁷, cyano, amino, amido or halo, wherein R⁷ is H, alkyl, arylalkyl or a protecting group;
X¹ is X³, —O—X⁴—, or —N(R⁸)—X⁴—, wherein X³ is alkyl (e.g., CH₂), X⁴ is a bond or alkyl, and R⁸ is H, alkyl, arylalkyl or acyl; and
X² is CR¹ or N.

Compounds of the formula (IV) include compounds of the formula (IVa):

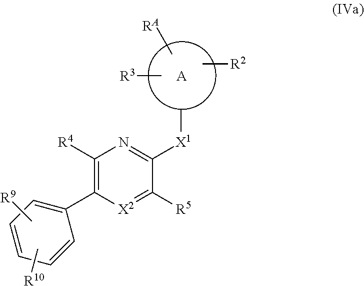

or a pharmaceutically acceptable salt, polymorph, prodrug, solvate or clathrate thereof.

In compounds of formula (IV) or (IVa), X² can be N. Or alternatively, or in combination, in the compounds of formula (IV) or (IVa), X² can be CR¹, wherein R¹ can be H. Alternatively, or in combination, in the compounds of formula (IV) or (IVa), R¹—R³ can each independently H, alkyl (e.g., 1 to 6 carbon atoms (C₁-C₆), such as methyl, ethyl, n-propyl, n-butyl, n-pentyl, n-hexyl, n-heptyl, n-octyl, isopropyl, iso-butyl, sec-butyl, t-butyl, neopentyl, isopentyl, and 2,2-dimethylpropyl groups), OR⁷ (wherein R⁷ is H, alkyl, arylalkyl (such as benzyl) or a protecting group or halo, such as fluoro (F), bromo (Br) or chloro (Cl)). Alternatively, or in combination, in the compounds of formula (IV) or (IVa), R⁵ can be cyano (C≡N) or amido (e.g., —C(O)NR₂, wherein each R can be H, alkyl, arylalkyl or arylalkyl). Alternatively, or in combination, in the compounds of formula (IV) or (IVa), R⁴ can be H or alkyl (e.g., 1 to 6 carbon atoms (C₁-C₆), such as methyl, ethyl, n-propyl, n-butyl, n-pentyl, n-hexyl, n-heptyl, n-octyl, isopropyl, iso-butyl, sec-butyl, t-butyl, neopentyl, isopentyl, and 2,2-dimethylpropyl groups). For example, in the compounds of the formula (IV) or (IVa), R⁴ can be H. Alternatively, or in combination, in the compounds of formula (IV) or (IVa), X¹ can be —N(R⁸)—X⁴—. For example, X⁴ can be a bond, such that the X¹ becomes a group of the formula —N(R⁸)—, such that the nitrogen atom bridges the ring comprising the groups R¹—R³ and the ring comprising R⁴ and R⁵ as follows:

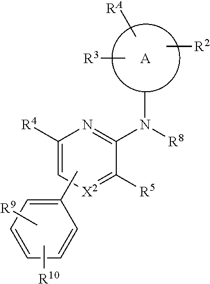

In the compounds of formula (IV) or (IVa), R⁸ can be H, alkyl (e.g., 1 to 6 carbon atoms (C₁-C₆), such as methyl, ethyl, n-propyl, n-butyl, n-pentyl, n-hexyl, n-heptyl, n-octyl, isopropyl, iso-butyl, sec-butyl, t-butyl, neopentyl, isopentyl, and 2,2-dimethylpropyl groups) or acyl (e.g., —C(O)R, wherein R is H, alkyl, aryl or arylalkyl).

Examples of the groups represented by the groups represented by A include, but are not limited to:

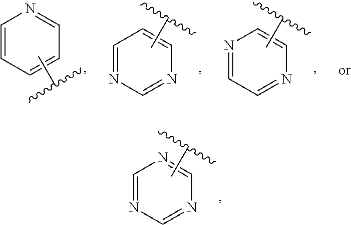

wherein each group can be substituted with R^{A}, R², and R³.

Also contemplated herein are compounds of the formulae:

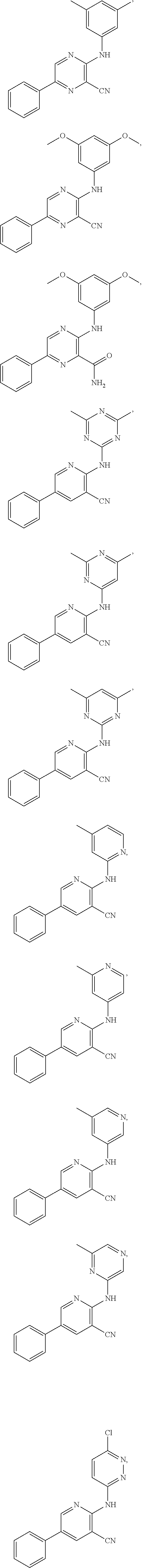

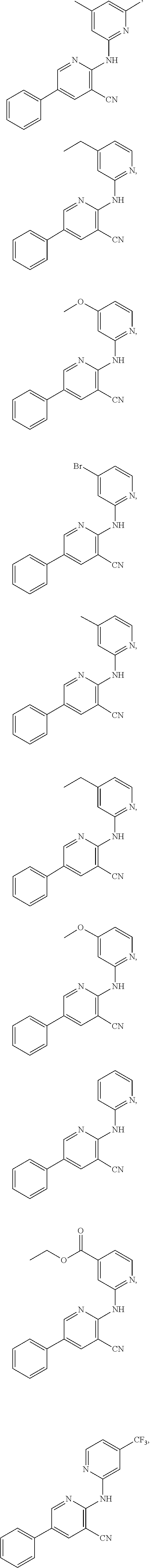

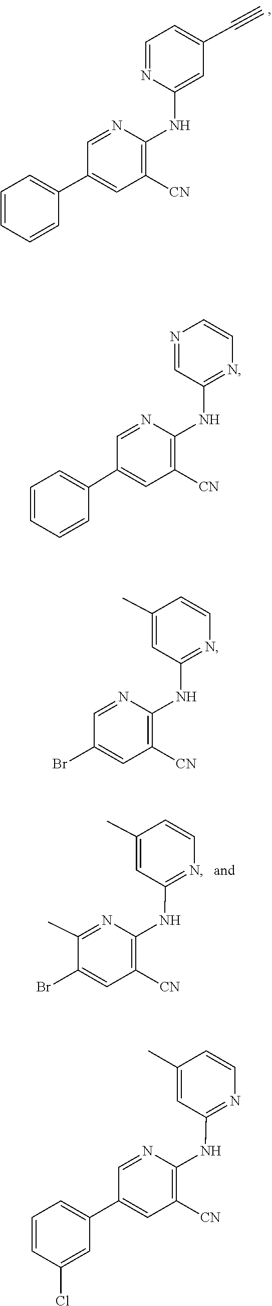

or a pharmaceutically acceptable salt, polymorph, prodrug, solvate or clathrate thereof.

While not wishing to be bound by any specific theory, it is believed that the compounds disclosed herein can be in equilibrium with a cyclized form of the compound. Thus, for example, compounds of the formula (III) where R⁵ is CN and A is of the formula

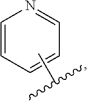

can be in equilibrium with compounds of the formula:

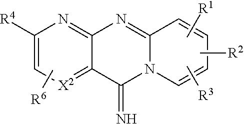

The term “substituted” or “substituent” as used herein refers to a group that can be or is substituted onto a molecule or onto another group (e.g., on an aryl or an alkyl group). Examples of substituents include, but are not limited to, a halogen (e.g., F, Cl, Br, and I), OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, —(CH₂)₀₋₂P(O)(OR)₂, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)C(O)OR, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein each R can be, independently, hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or two R groups bonded to a nitrogen atom or to adjacent nitrogen atoms can together with the nitrogen atom or atoms form a heterocyclyl, which can be mono- or independently multi-substituted.

The terms “alkyl” and “alkylene” as used herein refer to substituted or unsubstituted straight-chain and branched alkyl groups and cycloalkyl groups having from 1 to 40 carbon atoms (C₁-C₄₀), 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight-chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “cycloalkylalkyl” as used herein refers to substituted or unsubstituted alkyl groups as defined herein in which a hydrogen or carbon bond of an alkyl group as defined herein is replaced with a bond to a cycloalkyl group as defined herein. Representative cycloalkylalkyl groups include, but are not limited to, cyclopentylalkyl.

The term “alkylcycloalkyl” as used herein refers to substituted or unsubstituted cycloalkyl groups as defined herein in which a hydrogen of a cycloalkyl group as defined herein is replaced with a bond to an alkyl group as defined herein. Representative alkylcycloalkyl groups include, but are not limited to, alkylcyclopropyl.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. An example of an “acyl” group is an alkylenecarbonyl group (e.g., -alkylene-C(O)—) and alkylcarbonyl (e.g., —C(O)alkyl). When the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “heterocyclylcarbonyl” is an example of an acyl group that is bonded to a substituted or unsubstituted heterocyclyl group, as the term “heterocyclyl” is defined herein. An example of a heterocyclylcarbonyl group is a prolyl group, wherein the prolyl group can be a D- or an L-prolyl group.

The term “aryl” as used herein refers to substituted or unsubstituted cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” or “heterocyclo” as used herein refers to substituted or unsubstituted aromatic and non-aromatic ring compounds containing 3 or more ring members, of which, one or more (e.g., 1, 2 or 3) is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆), 3 to 5 carbon atoms (C₃-C₅) or 6 to 8 carbon atoms (C₆-C₈). A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. Representative heterocyclyl groups include, but are not limited to pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups. Examples of indolinonyl groups include groups having the general formula:

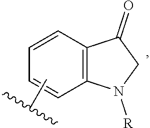

wherein R is as defined herein.
Examples of isoindolinonyl groups include groups having the general formula:

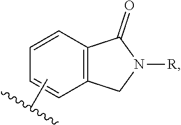

wherein R is as defined herein.
Examples of benzoxazolinyl groups include groups having the general formula:

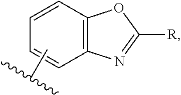

wherein R is as defined herein.
Examples of benzthiazolinyl groups include groups having the general formula:

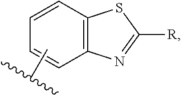

wherein R is as defined herein.
In some embodiments, the group R in benzoxazolinyl and benzthiazolinyl groups is an N(R)₂ group. In some embodiments, each R is hydrogen or alkyl, wherein the alkyl group is substituted or unsubstituted. In some embodiments, the alkyl group is substituted with a heterocyclyl group (e.g., with a pyrrolidinyl group).

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heterocyclylalkoxy” as used herein refers to alkyl groups as defined herein in which a hydrogen or carbon bond of an alkyl group as defined herein is replaced with a bond to a heterocyclyl group as defined herein and the alkyl group is attached to an oxygen. Representative heterocyclylalkoxy groups include, but are not limited to, —O—(CH₂)_{q}heterocyclyl, wherein q is an integer from 1 to 5. In some embodiments, heterocyclylalkoxy groups include —O—(CH₂)_{q}morpholinyl such as —O—CH₂CH₂-morpholine.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as defined herein. Examples of linear alkoxy groups include, but are not limited to, methoxy, ethoxy, propoxy, butoxy, pentyloxy, hexyloxy, and the like. Examples of branched alkoxy include, but are not limited to, isopropoxy, sec-butoxy, tert-butoxy, isopentyloxy, isohexyloxy, and the like. Examples of cyclic alkoxy include, but are not limited to, cyclopropyloxy, cyclobutyloxy, cyclopentyloxy, cyclohexyloxy, and the like. An alkoxy group can include one to about 12-20 or about 12-40 carbon atoms bonded to the oxygen atom, and can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein R is defined herein, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is defined herein, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

An example of a “alkylamino” is —NH-alkyl and —N(alkyl)₂.

An example of a “cycloalkylamino” group is —NH-cycloalkyl and —N(cycloalkyl)₂.

An example of a “cycloalkyl heterocycloamino” group is —NH-(heterocyclo cycloalkyl), wherein the heterocyclo group is attached to the nitrogen and the cycloalkyl group is attached to the heterocyclo group.

An example of a “heterocyclo cycloamino” group is —NH-(cycloalkyl heterocycle), wherein the cycloalkyl group is attached to the nitrogen and the heterocyclo group is attached to the cycloalkyl group.

The terms “halo,” “halogen,” and “halide”, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)₂ and the like.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 21st ed., Lippincott Williams & Wilkins, 2006, e.g., Chapter 38, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

The term “polymorph” generally refers to crystalline materials that have the same chemical composition but different molecular packing. The term “crystalline salt” includes crystalline structures with the same chemical materials but incorporating acid or base addition salts within the molecular packing of the crystalline structure.

The term “clathrate” generally refers to a compound in which molecules of one component (e.g., solvent) are physically trapped within the crystal structure of another.

The term “protecting group” refers to groups that prevent reaction at, among other groups, a hydroxyl group. Examples of suitable protecting groups include, but are not limited to silyl protecting groups (e.g., trimethylsilyl, t-butyldimethylsilyl, and t-butyl diphenylsilyl), tetrahydropyranyl protecting groups, ethoxyethyl protecting groups, benzyl protecting groups, naphthylmethyl protecting groups, p-methoxybenzyl ethers, and the like. See Peter G. M Wuts and Theodora W. Greene, Greene's Protective Groups in Organic Synthesis (4^{th} ed. 2007) for other commonly-used protecting groups for hydroxyl groups.

Pharmaceutical compositions comprising one or more compounds as described herein (e.g., a compound of the formula (I)) and one or more pharmaceutically acceptable carriers, diluents (e.g., fillers used to, among other things, increase weight and improve content uniformity in tablets, including starches, hydrolyzed starches, partially pregelatinized starches; other examples of diluents include anhydrous lactose, lactose monohydrate, and sugar alcohols such as sorbitol, xylitol and mannitol), excipients or combinations thereof. A “pharmaceutical composition” refers to a chemical or biological composition suitable for administration to a subject (e.g., mammal). Such compositions may be specifically formulated for administration via one or more of a number of routes including, but not limited to, buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. In addition, administration can be by means of capsule, drops, foams, gel, gum, injection, liquid, patch, pill, porous pouch, powder, tablet, or other suitable means of administration.

Also contemplated herein are pharmaceutical compositions comprising any compound described herein and at least one pharmaceutically acceptable excipient that is part of a nanoparticle, a liposomal or an exosomal formulation.

A “pharmaceutical excipient” or a “pharmaceutically acceptable excipient” comprises a carrier, sometimes a liquid, in which an active therapeutic agent is formulated. The excipient generally does not provide any pharmacological activity to the formulation, though it may provide chemical and/or biological stability, and release characteristics. Examples of suitable formulations can be found, for example, in Remington, The Science And Practice of Pharmacy, 20th Edition, (Gennaro, A. R., Chief Editor), Philadelphia College of Pharmacy and Science, 2000, which is incorporated by reference in its entirety.

As used herein “pharmaceutically acceptable carrier” or “excipient” includes any and all solvents, dispersion media, coatings, antibacterial and antifungal agents, isotonic and absorption delaying agents that are physiologically compatible. In one embodiment, the carrier is suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Pharmaceutical compositions may be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In some cases isotonic agents can be included in the pharmaceutical compositions, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the compounds described herein can be formulated in a time-release formulation, for example in a composition that includes a slow-release polymer. The active compounds can be prepared with carriers that will protect the compound against rapid release, such as a controlled-release formulation, including implants and microencapsulated delivery systems. Biodegradable, biocompatible polymers may be used, such as ethylene vinyl acetate, polyanhydrides, polyglycolic acid, collagen, polyorthoesters, polylactic acid and polylactic, and polyglycolic copolymers (PLG). Many methods for the preparation of such formulations are known to those skilled in the art.

Oral forms of administration are also contemplated herein. The pharmaceutical compositions may be orally administered as a capsule (hard or soft), tablet (film-coated, enteric-coated or uncoated), powder or granules (coated or uncoated) or liquid (solution or suspension). The formulations may be conveniently prepared by any of the methods well-known in the art. The pharmaceutical compositions may include one or more suitable production aids or excipients including fillers, binders, disintegrants, lubricants, diluents, flow agents, buffering agents, moistening agents, preservatives, colorants, sweeteners, flavors, and pharmaceutically compatible carriers.

For each of the recited embodiments, the compounds can be administered by a variety of dosage forms as known in the art. Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds, which can be included by admixture, are, for example, medically inert ingredients (e.g., solid and liquid diluent), such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The amount of active compound in a composition according to various embodiments may vary according to factors such as the disease state, age, gender, weight, patient history, risk factors, predisposition to disease, administration route, and pre-existing treatment regime (e.g., possible interactions with other medications). Dosage regimens may be adjusted to provide the optimum response. For example, a single bolus may be administered, several divided doses may be administered over time, or the dose may be proportionally reduced or increased as indicated by the exigencies of the situation. Desirably, a pharmaceutically effective amount is provided, and the amount is sufficient to provide a therapeutic effect to a subject having inflammation, such as inflammation associated with a disease or a disorder, or a prophylactic effect to a subject at risk for developing inflammation associated with a disease or disorder.

“Dosage unit form,” as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals. In therapeutic use for treatment of conditions in mammals (e.g., humans) for which the compounds of the various embodiments described herein, or an appropriate pharmaceutical composition thereof are effective, the compounds of the various embodiments described herein may be administered in an effective amount. The dosages as suitable for this invention may be a composition, a pharmaceutical composition or any other compositions described herein.

The dosage can be administered once, twice, or thrice a day, although more frequent dosing intervals are possible. The dosage may be administered every day, every 2 days, every 3 days, every 4 days, every 5 days, every 6 days, and/or every 7 days (once a week). In one embodiment, the dosage may be administered daily for up to and including 30 days, preferably between 7-10 days. In another embodiment, the dosage may be administered twice a day for 10 days. If the patient requires treatment for a chronic disease or condition, the dosage may be administered for as long as signs and/or symptoms persist. The patient may require “maintenance treatment” where the patient is receiving dosages every day for months, years, or for life. In addition, the composition of this invention may be to effect prophylaxis of recurring symptoms. For example, the dosage may be administered once or twice a day to prevent the onset of symptoms in patients at risk, especially for asymptomatic patients.

The compositions described herein may be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred routes of administration are buccal and oral. The administration can be local, where the composition is administered directly, close to, in the locality, near, at, about, or in the vicinity of, the site(s) of disease, e.g., inflammation, or systemic, wherein the composition is given to the patient and passes through the body widely, thereby reaching the site(s) of disease. Local administration can be administration to the cell, tissue, organ, and/or organ system, which encompasses and/or is affected by the disease, and/or where the disease signs and/or symptoms are active or are likely to occur. Administration can be topical with a local effect; composition is applied directly where its action is desired. Administration can be enteral wherein the desired effect is systemic (non-local), composition is given via the digestive tract. Administration can be parenteral, where the desired effect is systemic, composition is given by other routes than the digestive tract.

The term “treating,” as used herein, encompasses therapeutic (e.g., a subject with signs and symptoms of a disease state being treated) and prophylaxis. Prophylaxis and prophylactic encompass prevention and inhibition or delay of progression of a disease state.

The term “pharmaceutically effective amount” as used herein, refers to that amount of one or more compounds of the various embodiments described herein (e.g. a compound of the formula (I)) that elicits a biological or medicinal response in a tissue system, animal or human, that is being sought by a researcher, veterinarian, medical doctor or other clinician, which includes alleviation of the signs or symptoms of the disease or disorder being treated. In some embodiments, the pharmaceutically effective amount is that which may treat or alleviate the disease, signs or symptoms of the disease at a reasonable benefit/risk ratio applicable to any medical treatment. However, it is to be understood that the total daily usage of the compounds and compositions described herein may be decided by the attending physician within the scope of sound medical judgment. The specific pharmaceutically effective dose level for any particular patient will depend upon a variety of factors, including the condition being treated and the severity of the condition; activity of the specific compound employed; the specific composition employed; the age, body weight, general health, gender and diet of the patient; the time of administration, route of administration, and rate of excretion of the specific compound employed; the duration of the treatment; drugs used in combination or coincidentally with the specific compound employed; and like factors well known to the researcher, veterinarian, medical doctor or other clinician. It is also appreciated that the pharmaceutically effective amount can be selected with reference to any toxicity, or other undesirable side effect, that might occur during administration of one or more of the compounds described herein.

The disclosure also relates to methods for treating a tumor comprising administering one or more compounds described herein or the pharmaceutical compositions described herein, to a subject in need thereof. For example, the tumor can be a KEAP1 mutant tumor.

The disclosure also relates to methods for treating tumors with constitutive activation of the Nrf2 pathway (e.g., because of mutations in Keap1, NFE2L2 or epigenetic changes), such as small cell lung cancer (NSCLC), comprising administering one or more compounds described herein or the pharmaceutical compositions described herein, to a subject in need thereof.

Nrf2 has disparate roles in anti-viral immunity, but for certain viruses (e.g., hepatitis, dengue, Marburg, herpes, and influenza), Nrf2 inhibition can be beneficial. Some virus proteins are able to directly bind Keap1 to activate Nrf2 to confer a survival advantage to the virus, as is the case with the hemorrhagic fever-inducing Marburg virus. Nrf2-null mice have improved survival rates and reduced viral titers following infection with Marburg virus. In Dengue virus infected-mice, Nrf2 activation upregulated the proinflammatory protein CLEC5A which led to downstream inflammation, and Nrf2-null mice were protected from this inflammatory response. In human cells and in in vivo infection of mice, Nrf2 impairs innate antiviral immune responses against two herpes viruses, though the mechanism varied between species. See, e.g., Schaedler, S. et al. Hepatitis B virus induces expression of antioxidant response element-regulated genes by activation of Nrf2. J. Biol. Chem. 285, 41074-41086 (2010); Ivanov, A. V. et al. Hepatitis C virus proteins activate NRF2/ARE pathway by distinct ROS-dependent and independent mechanisms in HUH7 cells. PLoS One 6, e24957 (2011); Sugiyama, K. et al. Prominent steatosis with hypermetabolism of the cell line permissive for years of infection with hepatitis C virus. PLoS One 9, e94460 (2014); Murakami, Y. et al. Dual effects of the Nrf2 inhibitor for inhibition of hepatitis C virus and hepatic cancer cells. BMC Cancer 18, (2018); Olagnier, D. et al. Nrf2 negatively regulates STING indicating a link between antiviral sensing and metabolic reprogramming. Nat. Commun. 9, (2018); Gunderstofte, C. et al. Nrf2 Negatively Regulates Type I Interferon Responses and Increases Susceptibility to Herpes Genital Infection in Mice. Front. Immunol. 10, 2101 (2019); Page, A. et al. Marburgvirus Hijacks Nrf2-Dependent Pathway by Targeting Nrf2-Negative Regulator Keap1. Cell Rep. 6, 1026-1036 (2014); Kesic, M. J., Simmons, S. O., Bauer, R. & Jaspers, I. Nrf2 expression modifies influenza A entry and replication in nasal epithelial cells. Free Radic. Biol. Med. 51, 444-453 (2011); and Cheng, Y. L. et al. Activation of Nrf2 by the dengue virus causes an increase in CLECSA, which enhances TNF-α production by mononuclear phagocytes. Sci. Rep. 6, (2016), all of which are incorporated by reference as if set forth herein in their entirety.

Nrf2 has also been implicated in autoimmune disorders, including allergies. See, e.g., Klemm, P. et al. Nrf2 expression driven by Foxp3 specific deletion of Keap1 results in loss of immune tolerance in mice. Eur. J. Immunol. 00, 1-10 (2019); Wang, J., Liu, P., Xin, S., Wang, Z. & Li, J. Nrf2 suppresses the function of dendritic cells to facilitate the immune escape of glioma cells. Exp. Cell Res. 360, 66-73 (2017); and Rockwell C E, Zhang M, Fields P E, Klaassen C D. J Immunol. 2012 Feb. 15; 188(4):1630-7. Thus, Nrf2 inhibition can be beneficial in the treatment of autoimmune disorders.

The disclosure therefore also relates to methods for treating viral infections (e.g., influenza) and autoimmune disorders (e.g., allergies), comprising administering one or more compounds described herein or the pharmaceutical compositions described herein, to a subject in need thereof.

### EXAMPLES

The present disclosure can be better understood by reference to the following examples which are offered by way of illustration. The present invention is not limited to the examples given herein.

### Materials and Methods

### Synthesis

Compound 1 was synthesized as described in Advanced Synthesis & Catalysis 356:1811-22 (2014), which is incorporated by reference as if fully set forth herein:

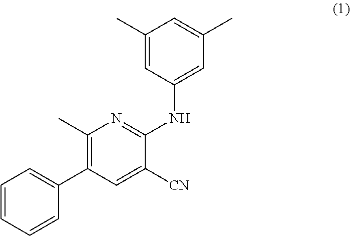 [SMILES: Cc1cc(C)cc(Nc2nc(C)c(-c3ccccc3)cc2C#N)c1]

Compound 1 was dissolved in DMSO to make a 50 mmol/L stock concentration and diluted to the listed concentrations for each experiment. An equivalent amount of DMSO was used as a vehicle control. The purity of the compound used in these studies was confirmed to be >98% by using gas chromatography with flame-ionization detection.

### Cell Culture

A NRF2/ARE luciferase reporter stably transfected into MCF7 cells were purchased from Signosis (Santa Clara, CA). Cells were cultured in DMEM+10% FBS+1% Pen/Strep (Corning Cellgro, Mediatech, Manassas, VA). A549, H460, A427, MCF7, MCF10A, Jurkat cells were purchased from ATCC and cultured at recommended conditions. A549 cells were maintained in F12K+10% FBS+1% Pen/Strep. H460 cells were maintained in RPMI+10% FBS+1% Pen/Strep. A427 cells were maintained in DMEM/F12+10% FBS+1% Pen/Strep. MCF10A cells were cultured in DMEM/F12 supplemented with 5% equine serum (Gemini Bio), 0.29 M sodium bicarbonate solution (Sigma-Aldrich), 10 mM Hepes (Sigma-Aldrich), 1% glutamine (Sigma-Aldrich), 10 μg/mL insulin (Sigma-Aldrich), 20 ng/mL EGF (Sigma-Aldrich), 1 mg/mL hydrocortisone (Sigma-Aldrich), 150 μg/mL cholera toxin (Sigma-Aldrich), and 1% Pen/Strep. Jurkat cells were maintained in RPMI+10% FBS+1% Pen/Strep. Keap1 CRISPR KO and βTrCP CRISPR KO Jurkat cells were generated as described in J Pharmacol Exp Ther 361: 259-67 (2017). The FBS concentration was reduced to 1% when treated with inhibitors.

### Nrf2 Activity Luciferase Reporter Assay

Ten thousand NRF2/ARE reporter cells were plated in a 96 well plate (white) with 1% FBS in the media. Cells were treated with different concentrations of compound 1 for 24 hours. tert-butylhydroquinone (tBHQ) was added 1 hour after compound 1 to activate the Nrf2 pathway. Cell viability was detected by Celltiter-fluor (Promega) and luciferase activity was detected by Steady-glo (Promega) using Synergy Neo HTS multi-mode microplate reader (BioTek).

### RT-qPCR and Western Blotting

A549 cells were treated with compound 1 at indicated concentrations for 24 hours. Total RNA was isolated using a RNeasy Mini Kit (QIAGEN). RNA concentrations were determined using NanoDrop. For each sample, 500 ng RNA was used to synthesize cDNA with high capacity cDNA reverse transcription kits (AppliedBiosystems). Primers were ordered from IDT. AppliedBiosystems Fast SYBR Green Master Mix and the QuantStudio 7 Flex Real-Time PCR system were used to detect gene expression. The delta-delta Ct method was applied to calculate relative gene expression. Values were normalized to the reference gene GAPDH and expressed as fold change compared to DMSO treated samples.

### Western Blotting

A549 or MCF7 cells treated with compound 1 were lysed in RIPA buffer (5 M NaCl, 1 M Tris-Cl, pH 7.4, 0.5 M EDTA, 25 mM deoxycholic acid, 1% triton-X, 0.1% SDS) containing protease inhibitors (1 mM PMSF, 2 μg/ml aprotinin and 5 μg/ml leupeptin) added just prior to use. Protein concentrations were measured using the BCA assay (Sigma-Aldrich). 20 μg of protein were separated by 10% SDS-PAGE gels and transferred to nitrocellulose membranes. Nrf2 (Novus Biologicals, 1:1000), NQO1 (Abcam, 1:1000), HO-1 (Abcam, 1:1000), Keap1 (Cell Signaling Technology, 1:1000), NF-κB (Cell Signaling Technology, 1:1000), IκBα (Cell Signaling Technology, 1:1000) Cyclin G1 (Santa Cruz, 35 1:1000), β-TrCP (Cell Signaling Technology, 1:1000), STAT3 (Cell Signaling Technology, 1:1000), Ubiquitin (Cell Signaling Technology, 1:1000), Histone 3 (Cell Signaling Technology, 1:4000), GAPDH (Santa Cruz, 1:4000) and Vinculin (Cell Signaling Technology, 1:4000) primary antibodies were applied to detect the corresponding proteins. Secondary antibodies (anti-rabbit or anti-mouse linked to HRP, 1:1000) were purchased from Cell Signaling Technology. ECL Western blotting substrate (GE Healthcare Life Sciences, UK) was used to detect the signal. Images shown are representative of three independent experiments. Protein quantification was done using ImageJ.

### Reactive Oxygen Species (ROS) Assay

A549 cells were treated with compound 1 for 24 hours. DCFDA (10 μM) was added for 2 hrs as an ROS indicator and then cells were treated with tert-butyl hydroperoxide (tBHP, 250 μM) for 15 minutes before harvesting. Then cells were washed with PBS and trypsinized to a single cell suspension. The cell pellet was resuspended in PBS and analyzed using a flow cytometer (Accuri, BD) with the FLA-1 channel. 50,000 events were acquired for each sample. Mean fluorescence intensity (MFI) was calculated using FlowJo software.

### Cell Growth Assay

To evaluate cell proliferation in 2D culture, a MTT assay was performed. Cells were seeded in 96-well plates with 2000 cells/well in their corresponding growth media. Various cell lines were treated with compound 1 for 72 hrs. To assess cell growth in 3D culture, A549, H460 and A427 cells were plated in 0.6% soft agar for 7 days. A Cytation 3 imaging reader from BioTek with Gen5 3.04 software was used to quantify colonies. Seven pictures were taken every 100 μm and superimposed together by z-projection function. Colonies>50 μm in diameter were quantified for each cell line.

### Chemoresistance Assay

One thousand A549 cells/well were plated in 384 well plates and treated with compound 1 and chemotherapy drugs using a series of concentrations. Cell viability was detected by Celltiter-glo (Promega) after 72 hours of treatment. Data was normalized to the DMSO control and presented as a percentage of cell viability. Data represent three independent experiments.

### In Vivo Treatment Study

Male athymic nude mice (Harlan Laboratories, 6 week-old) were injected in the flank with 5×10⁶ A549 cells. Cell line authenticity and absence of pathogens in the A549 cells were confirmed before establishing the model (I DEXX BioAnalytics). Once the tumors reached 4 mm in diameter as measured by a caliper, mice were randomized into four groups (6 mice/group) and treated daily (M-F) i.p. with either vehicle (10% DMSO/10% Cremophor/80% saline), compound 1 (50 mg/kg, BID), carboplatin (5 mg/kg), or the combination of compound 1 and carboplatin. All mice were weighed twice a week, and tumors were measured twice a week using a caliper. All mice were sacrificed after 4 weeks of treatment and harvested for analysis of Nrf2 expression and cell proliferation.

### Immunohistochemistry

Tumors were harvested and fixed in 10% neutral buffered formalin. Sectioned were obtained for immunohistochemistry staining. Sodium citrate buffer (10 mM, Vector) was used for antigen retrieval. 3% hydrogen peroxide (15-minute incubation) was used to quench the endogenous peroxidase activity. Sections were stained with PCNA (1:200, Santa Cruz) or Nrf2 (1:200, Novus Biologicals) antibodies for 1 hour at room temperature or overnight at 4° C., respectively. Anti-mouse and anti-rabbit secondary antibodies conjugated to HRP were purchased from Cell Signaling Technology. Signal was detected using a DAB kit (Cell Signaling Technology) and sections were counterstained with hematoxylin (Vector).

### Statistical Analysis

The in vitro experiments were performed in triplicate and were repeated independently at least three times. Unless noted otherwise, data are presented as mean±SE. In vitro and in vivo results were analyzed using one-way ANOVA followed by a Tukey test or one-way ANOVA on ranks and the Dunn test if the data did not fit a normal distribution (Prism 6). Tumor volume curves were analyzed using two-way repeated measures ANOVA followed by Tukey's multiple comparisons test. A p value<0.05 was considered statistically significant.

### Example 1: Compound 1 Inhibits Nrf2 Transcriptional Activity and Suppresses the Expression of Genes Downstream of Nrf2

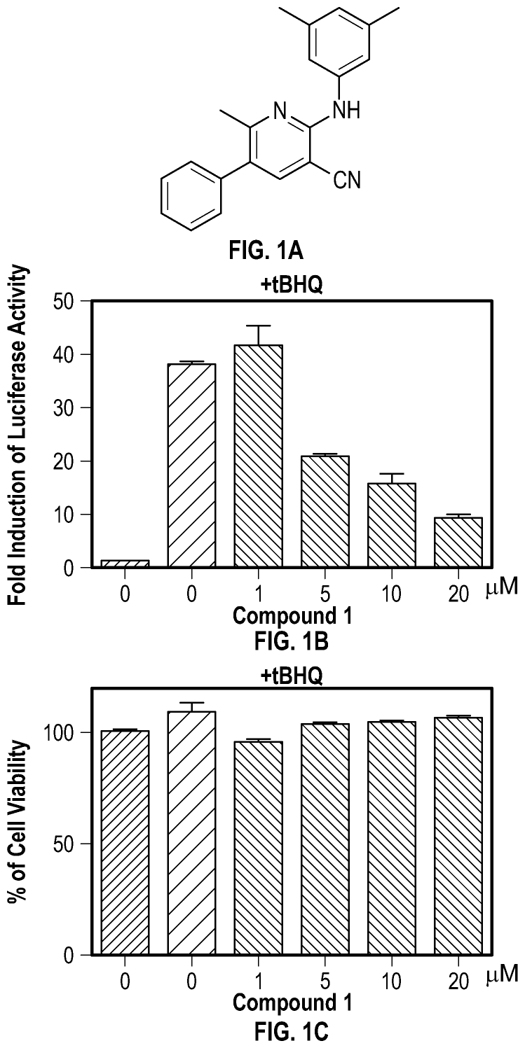

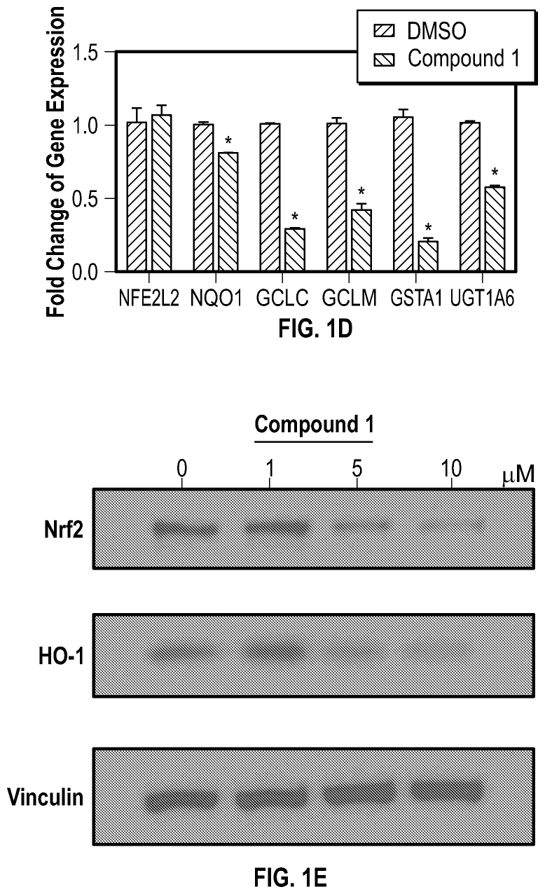

To identify Nrf2 inhibitors, approximately 600 small molecules synthesized at Michigan State University were screened using a cell-based luciferase reporter assay coupled with a cytotoxicity readout. A commercially available NRF2/ARE luciferase reporter stably expressed in MCF-7 cells was used as a tool for the initial screen. Hit compounds were further validated in KEAP1-mutant cell lines, such as A549 cells (human lung adenocarcinoma cells). MCF-7 cells (human breast cancer cells), stably transfected with a Nrf2 binding site and firefly luciferase coding region, were treated with library compounds at 10 μM for 24 hours. tert-Butylhydroquinone (tBHQ), a well-known Nrf2 activator, was added 1 hour after the library compounds to stimulate the activation of the Nrf2 pathway. Before the detection of luciferase activity, cell viability was measured using a cell-titer fluor assay. From the primary screen, Compound 1 was identified as a hit (FIG. 1A). Compound 1 inhibited Nrf2 transcriptional activity induced by tBHQ in a dose-dependent manner (FIG. 1B) without reducing cell viability (FIG. 1C).

Nrf2 regulates a wide variety of downstream cellular processes, such as detoxification, maintenance of redox homeostasis, and heme metabolism. To validate the inhibitory effects of compound 1 on the Nrf2 pathway in A549 cells, mRNA expression of targets downstream of Nrf2 was detected after 24 hours of treatment with compound 1: NAD(P)H dehydrogenase (quinone 1) (NQO1), glutamate-cysteine ligase catalytic subunit (GCLC), glutamate-cysteine ligase regulatory subunit (GCLM), Glutathione S-transferase 1A1 (GST1A1), and UDP-glucuronosyltransferase (UGT1A6). Compound 1 significantly (p<0.05) decreased the expression of all these downstream genes in A549 cells (FIG. 1D). compound 1 did not affect the mRNA expression of NFE2L2, the gene that encodes the Nrf2 protein (FIG. 1D). Furthermore, compound 1 inhibited Nrf2 pathway activity at the protein level, as the protein expression of heme oxygenase-1 (HO-1), another downstream target of Nrf2, was dose-dependently decreased after treatment (FIG. 1E).

### Example 2: Compound 1 Enhances Nrf2 Degradation Through the Proteasome

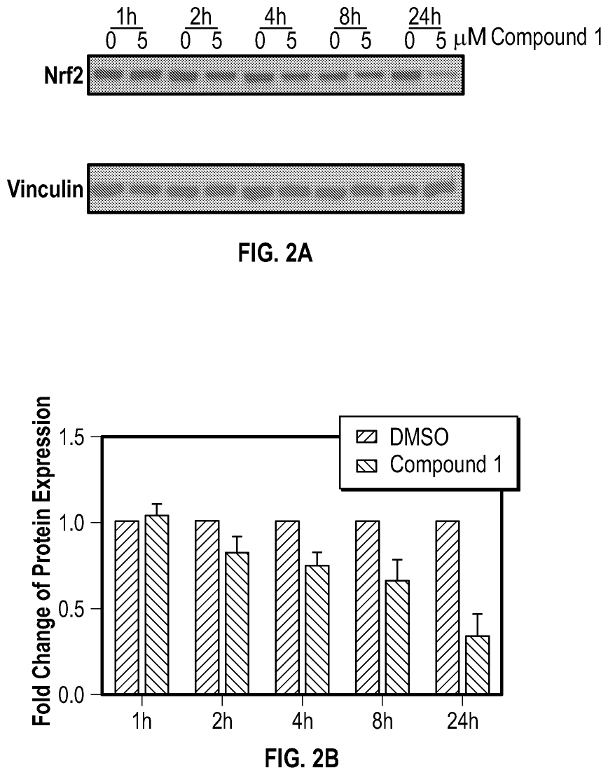

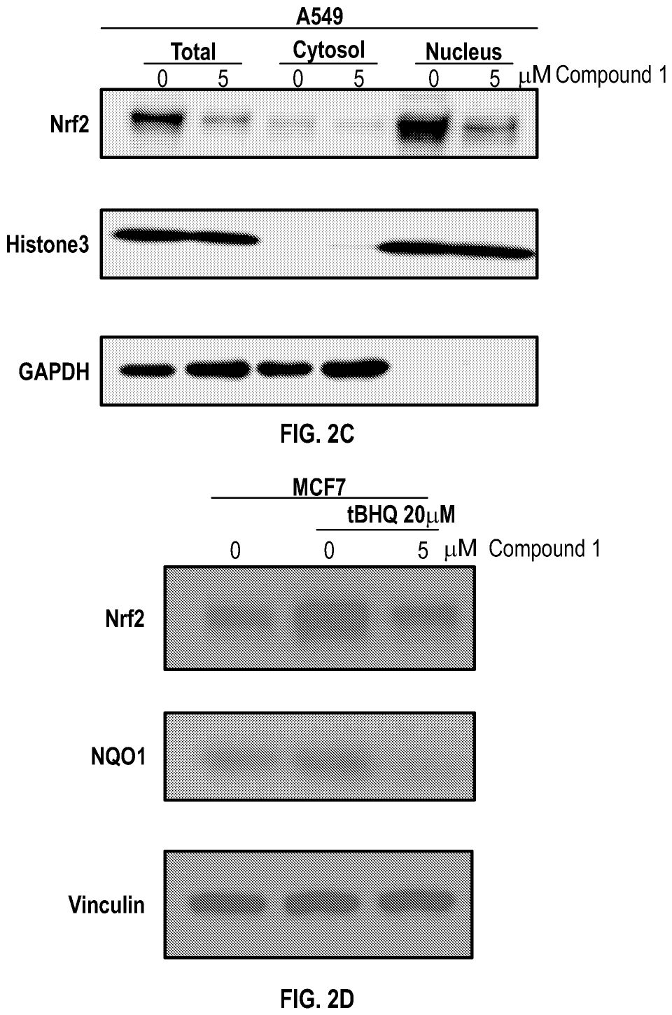

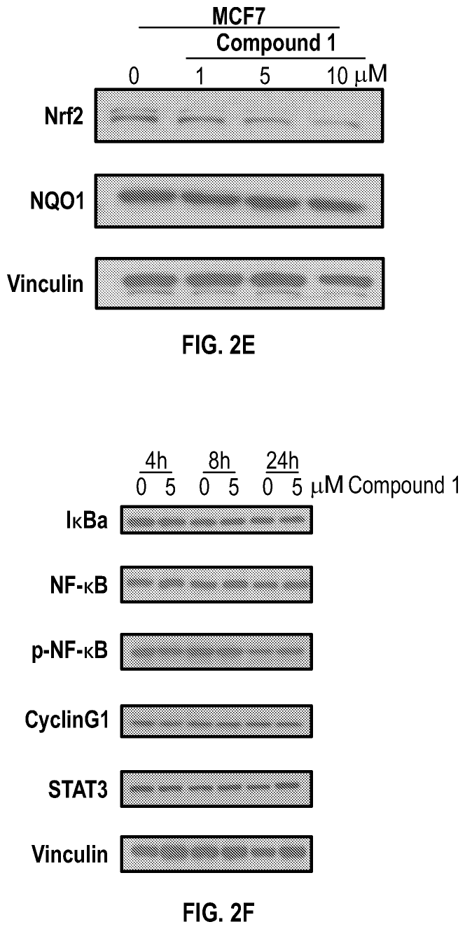

In A549 cells, Nrf2 is constitutively activated because of a mutation in the KEAP1 gene and is expressed at a higher level than in KEAP1 wildtype cells. compound 1 decreased the protein level of Nrf2 in a dose- (FIG. 1E) and time- (FIGS. 2A and B) dependent manner. The largest reduction in Nrf2 protein was found 24 hours post-treatment with compound 1, which is a different kinetic profile than other Nrf2 inhibitors such as brusatol. Nrf2 protein is mainly located in the nucleus in A549 cells because of its constitutive activation. Although the cytosolic expression of Nrf2 was very low, Compound 1 did reduce Nrf2 expression in both the cytosol and nucleus (FIG. 2C). In addition, the effects of compound 1 on Nrf2 protein level is independent of the Nrf2 mutation status. In MCF-7 cells, where Keap1 and Nrf2 are wild-type, compound 1 decreased both tBHQ-induced expression of Nrf2 (FIG. 2D) and basal expression of Nrf2 protein (FIG. 2E). NQO1, a downstream target of Nrf2, protein was also decreased after treatment (FIG. 2D). Notably, compound 1 did not alter the overall expression pattern of abundant cellular proteins (data not shown). compound 1 also did not change levels of NF-κB pathway proteins (IκBa, NF-κB, p-NF-κB) or other fast turnover proteins such as cyclinG1 and STAT3 (FIG. 2F).

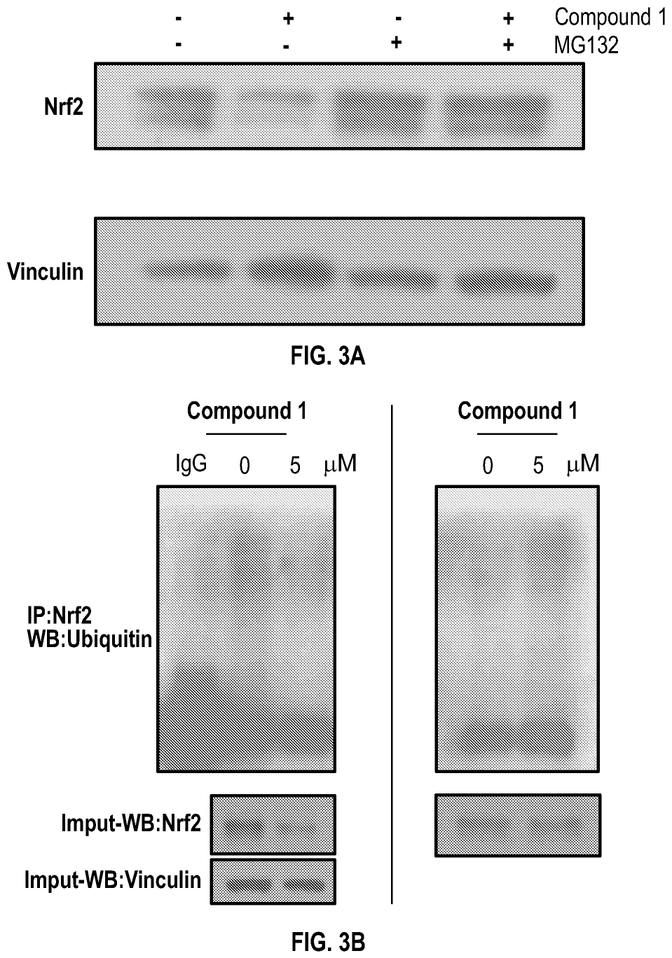

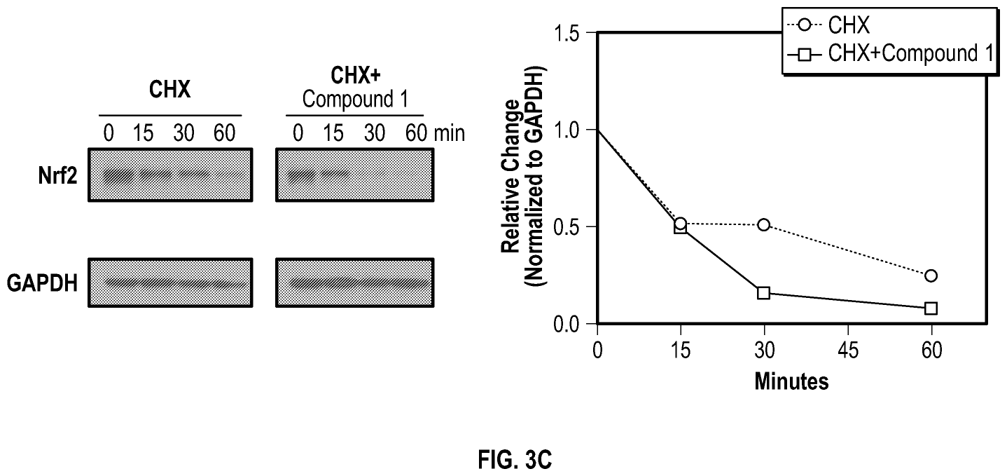

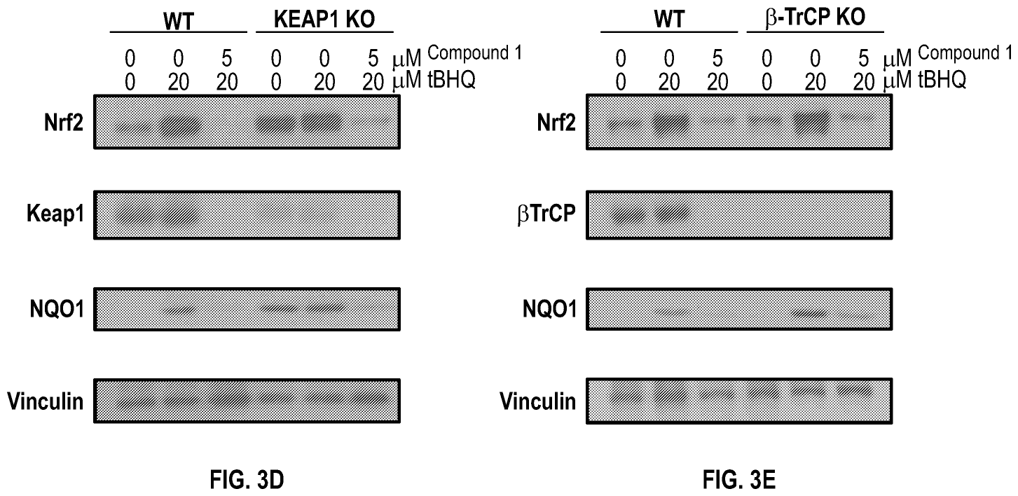

The protein level of Nrf2 is tightly balanced between synthesis and degradation. To investigate whether compound 1 affects the degradation of Nrf2 protein, A549 cells were treated with MG132, a proteasome inhibitor. MG132 blocked the decrease in protein expression of Nrf2 when treated with compound 1 (FIG. 3A), suggesting that compound 1enhances the protein degradation of Nrf2 via the proteasome system. Additionally, compound 1 increased the ubiquitination of Nrf2 (FIG. 3B). In this experiment, A549 cells were treated with either DMSO or compound 1 for 24 hours, and Nrf2 protein was pulled down using agarose beads. Ubiquitin level was then evaluated on the Nrf2 protein. Because compound 1 decreased Nrf2 protein level, no change in the ubiquitin level was observed when the same amount of total protein was loaded to compare between the DMSO control and treatment (FIG. 3B left). However, a higher level of ubiquitination of Nrf2 protein was present when the same amount of Nrf2 protein was loaded (FIG. 3B right). The cycloheximide chase assay is widely used to assess the stability of proteins. A549 cells were pretreated with DMSO or compound 1 for 4 hours. After washout with PBS, cells were treated with 1 μM cycloheximide for 0-60 minutes before being harvested to detect the protein level of Nrf2. At 30 min, Nrf2 protein levels were lower in cells pretreated with compound 1 and then cycloheximide compared to the DMSO pretreated controls, suggesting that compound 1 facilitates the decreased stability of Nrf2 protein (FIG. 3C). Keap1-Cullin3 and βTrCP-Cullin1 are two major pathways that lead to Nrf2 degradation (28,29). However, compound 1 enhances Nrf2 degradation in a largely Keap1- and βTrCP-independent manner, as compound 1 still reduced Nrf2 protein expression in Keap1 CRISPR KO and βTrCP CRISPR KO Jurkat cells (FIGS. 3C and D).

### Example 3: Compound 1 Dampens the Anti-Oxidative Response and Inhibits Cell Growth in Cancer Cells

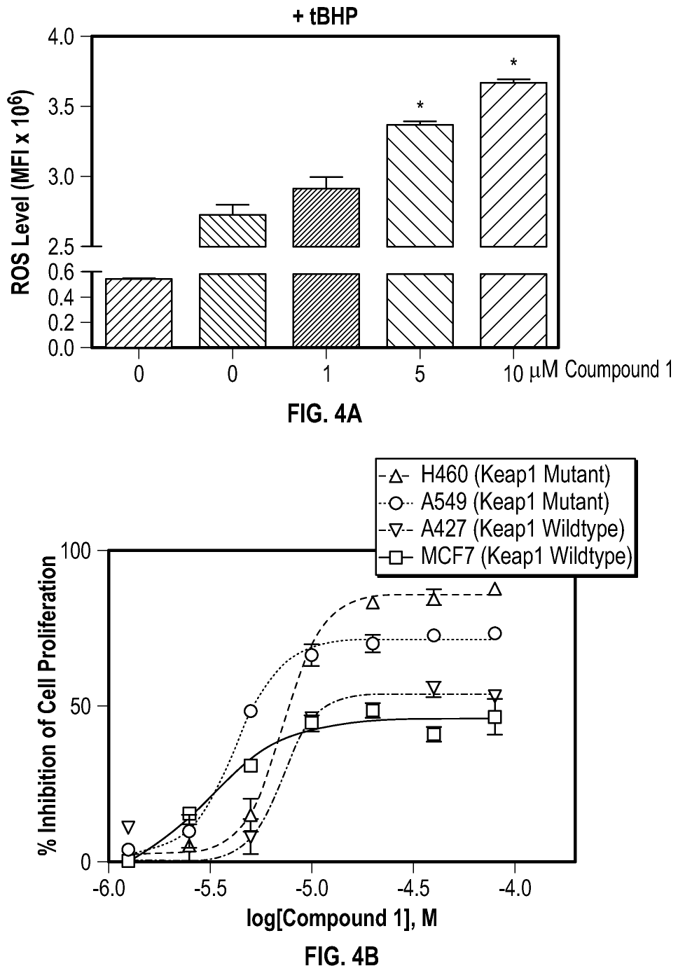

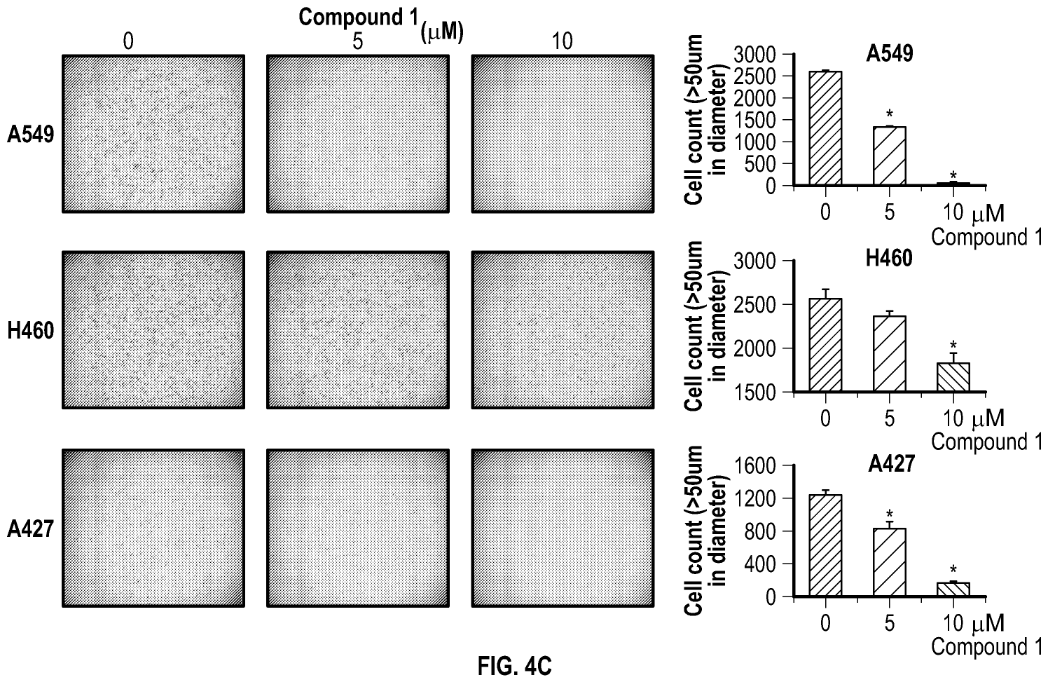

Nrf2 is a master regulator of anti-oxidative responses and plays critical roles in maintaining redox balancing. Nrf2 activators can attenuate oxidative stresses. To test the effects of compound 1 on the production of reactive oxygen species (ROS), A549 cells were treated with compound 1 for 24 hours and then stimulated with tert-butyl hydroperoxide (tBHP) for 15 minutes. compound 1 dose-dependently increased ROS production in A549 cells (FIG. 4A), which functionally validated the inhibitor as the anti-oxidative response regulated by the Nrf2 pathway was suppressed.

Besides its anti-oxidative properties, Nrf2 also contributes to tumor cell growth. Nrf2 not only regulates cell proliferation as a downstream mediator of several onco-proteins but also reprograms cellular metabolism to favor anabolic pathways. Cancer cells were treated with compound 1 for 72 hours and cell viability was detected using the MTT assay. compound 1 inhibited proliferation in various cancer cells, including both Keap1 mutant and Keap1 wild-type cells. Notably, Nrf2-addicted cancer cell lines (A549 and H460 with mutant Keap1) showed a higher sensitivity to compound 1 treatment compared to non-addicted cells (A427 and MCF-7 with wildtype Keap1). In MCF-10A cells (a non-tumorigenic epithelial cell line), compound 1 did not have any effects on cell viability at concentrations<20 μM (data not shown). The suppression of compound 1 on cell growth was also confirmed in 3D cell culture. Three different human lung cancer cell lines, including A549, H460 and A427 cells, were plated in 0.6% soft agar and treated with compound 1 for 7 days. Compound 1 significantly (p<0.05) suppressed the growth of all three lung cancer cells grown in soft agar in a dose-dependent manner (FIG. 4C).

### Example 4: Compound 1 Enhances the Sensitivity of Cancer Cells to Chemotherapeutic Drugs In Vitro and In Vivo

Drug resistance during chemotherapy remains a major obstacle in cancer treatment. Activation of the Nrf2 pathway is one mechanism that tumor cells hijack to induce chemoresistance. Nrf2 regulates a series of drug metabolizing enzymes and efflux transporters so that drug exposure could be lower in cancer cells with increased Nrf2 expression and activity. Moreover, Nrf2-induced chemoresistance can be redox-mediated. ROS-mediated apoptosis is a common mechanism of action for many chemotherapeutic agents. Cancer cells upregulate antioxidants to protect them from high level of ROS by turning on the Nrf2 pathway (34). The Nrf2 pathway can also induces drug resistance through the activation of autophagy.

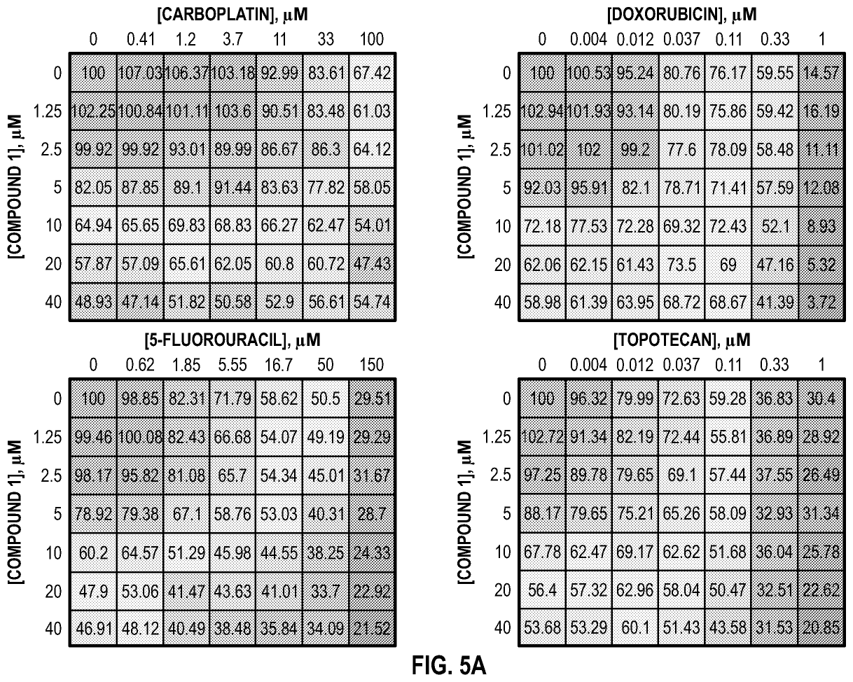

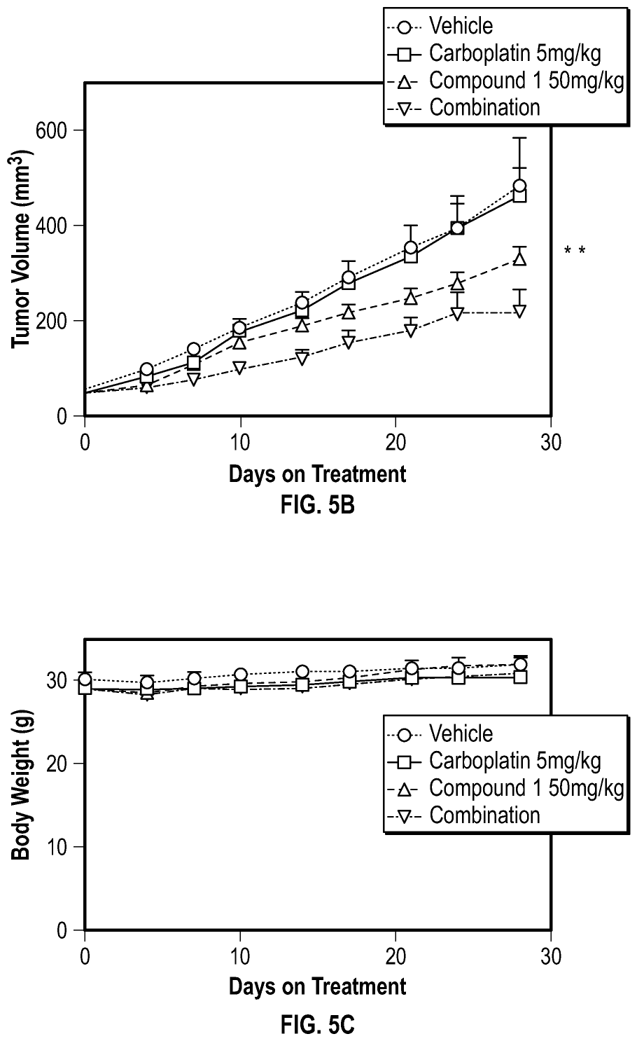

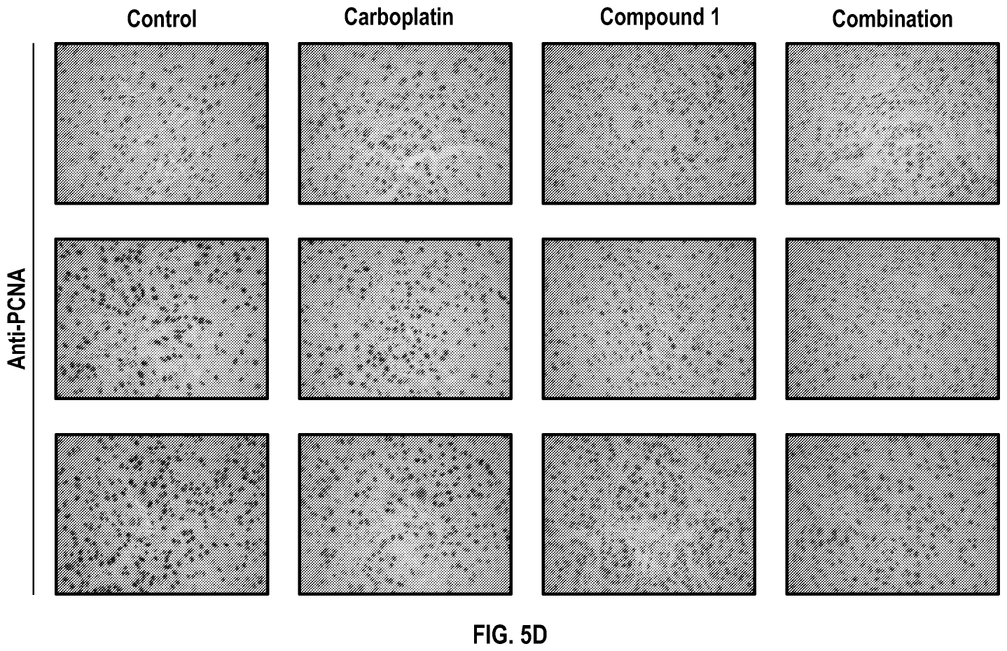

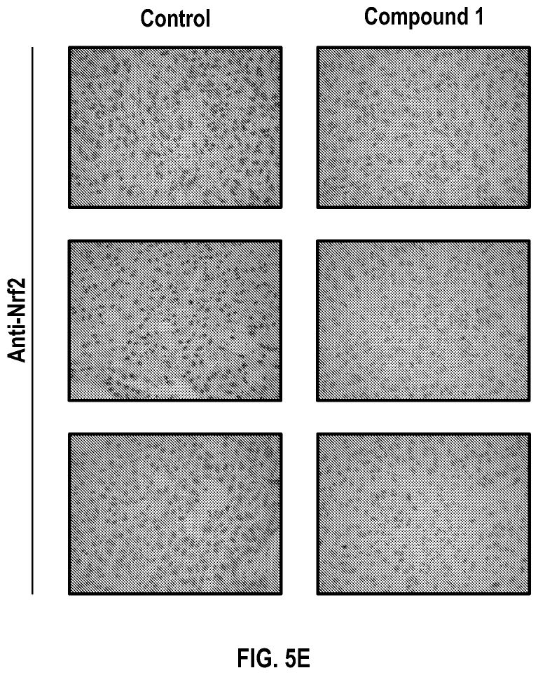

To test whether compound 1 could overcome the resistance to chemotherapies, A549 cells were treated with several commonly used chemotherapeutic agents in combination with compound 1. Additive effects of compound 1 were observed with all the chemotherapy drugs, including carboplatin, doxorubicin, 5-fluorouracil, and topotecan (FIG. 5A). Lower cell viability was induced by chemotherapeutic agents when used in combination with compound 1 compared to the drugs as single agents. Furthermore, enhanced efficacy was achieved when chemotherapy was combined with compound 1 in vivo. A subcutaneous xenograft model was established using A549 cells. Tumors 5 mm in diameter were treated with either vehicle, low dose carboplatin, Compound 1, or the combination for four weeks. Carboplatin, a standard of care chemotherapy for treatment of lung cancer, failed to arrest tumor growth at this low dose (5 mg/kg) when used alone (FIG. 5B). As a single agent, compound 1 appeared to slow tumor growth, but the change was not statistically significant. However, the combination of compound 1 and carboplatin significantly slowed tumor growth compared to the vehicle control (p<0.01) and carboplatin alone (p<0.05) (FIG. 5B). Compound 1 was generally well tolerated in the mice without inducing any changes on body weight over time (FIG. 5C). The combination therapy inhibited cell proliferation as indicated by a decrease in PCNA staining in the tumors (FIG. 5D). Consistent with the in vitro results, treatment with Compound 1 diminished the expression of Nrf2 protein in the tumors compared to the vehicle control group (FIG. 5E).

The Examples presented herein demonstrate the emergence of compound 1 as a novel Nrf2 pathway inhibitor. Compound 1 decreased Nrf2 protein in KEAP1 mutant cancer cells and suppressed the expression of targets genes downstream of Nrf2. These effects are likely because of enhanced degradation of Nrf2 through the proteasome system. Compound 1 not only inhibited the proliferation of cancer cells, especially Nrf2 addicted ones, but also sensitized lung cancer cells to chemotherapies. These results described a novel pharmacological tool compound to study this important signaling pathway and confirmed the relevance of using an Nrf2 pathway inhibitor in combination with chemotherapies in Nrf2 addicted lung cancers.

Compound 1 presents different features compared to other reported Nrf2 pathway inhibitors. Brusatol, the most potent known Nrf2 inhibitor, acts rapidly in reducing Nrf2 protein expression as the maximum reduction of Nrf2 occurs within 2-4 hours. The inhibition of Nrf2 by brusatol is also reversible as Nrf2 protein levels returned to basal levels within 8 hours. This fast-acting behavior is consistent with the reported mechanism of brusatol in inhibiting global protein synthesis. In contrast, it took 24 hours for compound 1 to reach peak inhibition. Compound 1 had no effect on Nrf2 transcripts. The different kinetics suggest that compound 1 regulates the Nrf2 pathway in a mechanism other than inhibiting the synthesis of genes or proteins. Additionally, the effect of compound 1 on Nrf2 protein was largely Keap1- and βTrCP-independent as compound 1 maintained its inhibitory effects on Nrf2 in Keapi and βTrCP knockout cells. This regulation is different than the compound clobetasol propionate, for example, which promotes βTrCP-dependent degradation of Nrf2. Compound 1, with different mechanisms, provides a new option to the list of pharmacological tools that inhibit the Nrf2 pathway.

Compound 1 showed promising selectivity for targeting the Nrf2 pathway. It did not alter the general expression pattern of enriched cellular proteins. It also did not regulate proteins in the NF-κB pathway, which has a similar mechanism of regulation as Nrf2. When treated with compound 1, Nrf2 addicted cells (Keap1 mutant) were more sensitive than Nrf2/Keap1 wild type cells. Notably, compound 1 did not affect proliferation of normal epithelial cells at concentrations that inhibited the growth of tumor cells. All these results suggest an appropriate therapeutic window for compound 1 to target the Nrf2 pathway in cancer cells without inducing global inhibitory effects.

Targeting the Nrf2 pathway has emerged as a new opportunity to treat tumors with constitutively activated Nrf2. However, patients may not benefit from Nrf2 inhibitors equally. It can be critical to determine which patient populations would benefit the most and how to maximize the utility of Nrf2 inhibition. For example, the effects of Nrf2 inhibitors may depend on the status of other genes. Targeting co-dependent vulnerabilities or synthetic lethal partners often enhances efficacy. Nrf2-mediated antioxidant activity can be upregulated by oncogenes, such as Kras, B-Raf, and Myc, to detoxify the increased ROS found in tumors, as genetic deletion of Nrf2 impairs tumorigenesis driven by activating Kras mutations. Loss of Nrf2 has also been shown to impair EGFR signaling, thus inhibiting cell proliferation in pancreatic cancer (41). In KRAS-mutant lung adenocarcinoma patients, KEAP1 mutations often accompany the loss of LKB1. LKB1-deficient cells with Nrf2 activation have enhanced cell survival and better maintenance of energetic and redox homeostasis in a glutamine-dependent manner. They are more sensitive to glutamine inhibitors.

As immunotherapies have become the first-line therapy in lung cancer, it is also critical to understand the effects of Nrf2 inhibitors on the immune system. The dual role of Nrf2 in cancer has focused on normal epithelial cells vs. cancer cells. In contrast, the effects of Nrf2 activity on immune cells within the tumor microenvironment have not been fully characterized. An unfavorable immune signature has been identified in advanced lung tumors from Nrf2 knockout mice in a carcinogen-induced mouse model of lung cancer. In contrast, other studies reported that Nrf2 activation promotes the polarization of macrophages to a M2 phenotypes and drives epithelial-mesenchymal transition. These differing results suggest that the effects of Nrf2 activation in the tumor microenvironment are likely context dependent.

### Synthetic Examples 5-80

### General Considerations

All manipulations were carried out under an inert dinitrogen atmosphere in an MBraun glovebox or using standard Schlenk techniques. Toluene was sparged with dinitrogen and passed over an activated alumina column prior to use. Tetrahydrofuran, n-hexane, and 1,4-dioxane were dried over sodium-benzophenone radical, refluxed, and distilled under dinitrogen prior to use. Ethanol was dried over magnesium, refluxed, and distilled under dinitrogen prior to use. p-Cymene was sparged with dinitrogen and distilled from CaH₂ prior to use. All deuterated NMR solvents were purchased from Cambridge Isotope Laboratories. Benzene-d6 was dried over CaH₂ and distilled under dinitrogen. CDCl₃ was dried over P₂O₅ and distilled under dinitrogen. Synthesis of tert-butylisonitrile was made according to the literature procedure and purified by distillation under dry dinitrogen. Synthesis of Ti(dpm)(NMe₂)₂ and Ti(NMe₂)₃/SiO₂⁷⁰⁰ was done according to the literature procedures.

Ti(NMe₂)₄ was purchased from Gelest and used as received. Palladium acetate was purchased from Strem and used as received. Tris(dibenzylideneacetone)dipalladium was purchased from Oakwood and used as received. BINAP was purchased from Alfa Aesar Chemicals and used as received. DMAPF was purchased from Sigma Aldrich and used as received. 1-phenyl-1-propyne and phenylacetylene were purchased from Combi-block and distilled from barium oxide prior to use.

### Abbreviations

1,1′-bis[bis(dimethylamino)phosphino]ferrocene=DMAPF, 4,5-Bis(diphenylphosphino)-9,9-dimethylxanthene=Xantphos, (2,2′-bis(diphenylphosphino)-1,1′-binaphthyl)=BINAP, N-bromosuccinimide=NBS, mCPBA=meta-chloroperoxybenzoic acid, 5,5-dimethyldipyrrolylmethane=H₂dpm

### Synthesis of Compounds

### General Procedure A: Synthesis of 2-amino-nicotinonitrile Derivatives via Titanium-Catalyzed Multicomponent Coupling

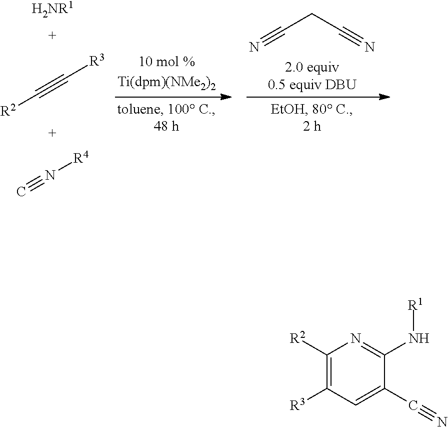

In the glovebox, a 15 mL pressure tube equipped with a stir bar was loaded with Ti(dpm)(NMe₂)₂ (32 mg, 0.1 mmol, 10 mol %) in dry toluene (2 mL). To the solution was added arylamine (1.0 mmol, 1.0 equiv), alkyne (1.0 mmol, 1.0 equiv), and tert-butylisonitrile (1.5 mmol, 1.5 equiv). The pressure tube was sealed with a Teflon screw cap and taken out of the glovebox. With stirring, the solution was heated for 48 h at 100° C. in a silicone oil bath. The pressure tube was cooled to room temperature. Then, the tube was charged with malononitrile (132 mg, 2.0 mmol), DBU (76 mg, 0.5 mmol), molecular sieves (200 mg), and ethanol (2 mL). The mixture was heated for 2-12 h at 80° C. in an oil bath. The crude product was purified by column chromatography (silica gel, hexanes:EtOAc 10:1) to afford the desired products.

### 2-((3,5-dimethylphenyl)amino)-5-phenylnicotinonitrile (1b)

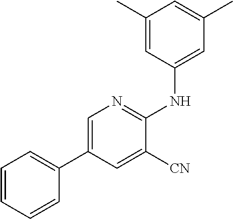 [SMILES: Cc1cc(C)cc(Nc2ncc(-c3ccccc3)cc2C#N)c1]

General procedure A was followed using 3,5-dimethylaniline (125 mL, 1.0 mmol, 1.0 equiv), phenylacetylene (110 mL, 1.0 mmol, 1.0 equiv), tert-butylisonitrile (170 mL, 1.5 mmol, 1.5 equiv), Ti(dpm)(NMe₂)₂ (32 mg, 0.1 mmol, 10 mol %), and 2 mL of dry toluene. The second step used malononitrile (132 mg, 2.0 mmol, 2.0 equiv), DBU (76 mg, 0.5 mmol, 0.5 equiv), molecular sieves (200 mg), 2 mL of ethanol and was heated for 2 h. Removal of solvent afforded product as a white solid (58 mg, 19%). M.p.: 114-115° C. ¹H NMR (CDCl₃, 500 MHz, 21° C.): 8.63 (s, 1H), 7.98 (s, 1H), 7.52-7.43 (m, 3H), 7.42-7.38 (m, 1H), 7.23 (s, 2H), 6.95 (s, 1H), 6.80 (s, 1H), 2.35 (s, 6H). ¹³C{¹} NMR (CDCl₃, 126 MHz, 21° C.): 155.34, 151.00, 139.78, 138.96, 138.71, 136.27, 129.39, 128.07, 127.56, 126.40, 126.22, 118.99, 116.61, 115.71, 21.61. LRMS (EI): calc'd: 299, found: 298.

Alternative method: A 15 mL pressure tube was charged with Ti(NMe₂)_{3/}SiO₂⁷⁰⁰ (320 mg), 2,6-dimethylphenylamidate (26 mg, 0.10 mmol), p-cymene (1 mL), and a Teflon coated stir bar. This mixture was stirred at room temperature for 5 min. Separately, a solution containing 3,5-dimethylaniline (242 mg, 2.0 mmol), CyNC (218 mg, 2.0 mmol), and phenylacetylene (408 mg, 4.0 mmol) in p-cymene was prepared (with a total volume of ˜2 mL). This solution was added to the contents of the pressure tube, which immediately resulted in a color change from pale yellow-orange to bright red. The tube was sealed and transferred from the glovebox to a preheated aluminum block (180° C.) where it was heated and stirred for 2 h. GC-FID of the crude 3CC reaction mixture at this point showed ˜90% yield of the 3 CC product. The tube was removed from heat and allowed to cool to room temperature before being opened in air. The following reagents were then added: 200 mg of activated 3 Å molecular sieves, 3 mL of dry EtOH, DBU (151 mg, 1 mmol), and malononitrile (264 mg, 4 mmol). The tube was once again sealed and heated to 80° C. in an oil bath, with stirring, for 2 h. After 2 h the reaction was cooled. The crude product could be identified by GC-MS as the targeted pyridine. The contents of the pressure tube were transferred to a round bottom flask, and the volatiles removed by rotary evaporation. This resulted in ˜2 mL of a viscous brown oil, which was purified by column chromatography (hexanes, gradient with 0-10% EtOAc, Al₂O₃ packing, product fluoresces under long-UV, Rf ˜0.5). From the column fractions, solvent was removed by rotary evaporation to yield the product as a waxy tan solid. This waxy solid was washed with hexanes to afford an off-white powder that was pure by several methods of characterization. Yield: 62 mg, 11%. Additionally, from this powder, X-ray quality crystals were grown from a solution of acetone and diethyl ether layered with hexane and stored at −20° C. overnight.

### 2-methyl-1-penten-3-yne (1c-I)

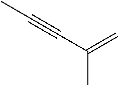

In the glovebox, a 100 mL, 1-neck Schlenk flask equipped with a stir bar was loaded with 2-methyl-1-buten-3-yne (4.78 g, 72.4 mmol) in dry THF (30 mL). The flask was placed inside a liquid nitrogen-cooled cold-well for 10 min. Then, the flask was removed from the cold-well, and n-butyllithium (2.5 M in hexane, 32 mL, 80 mmol) was added dropwise in 3 portions (10 mL each portion). The solution was cooled in the in the cold-well for 5 min between each portion. After the addition was completed, the mixture was stirred in the cold-well for 1 h. With stirring, iodomethane (11.3 g, 79.6 mmol) was added dropwise. The solution was allowed to warm to room temperature and stirred for 1 h. The flask was taken out of glovebox and water (30 mL) was added. The organic layer was collected and dried over Na₂SO₄. The product was obtained from fractional distillation (3.1 g, 38.8 mmol, 53.5%). ¹H NMR and ¹³C NMR were consistent with those previously reported. ¹H NMR (CDCl₃, 500 MHz, 21° C.): δ 5.17 (s, 1H), 5.12 (s, 1H), 1.92 (s, 3H), 1.84 (d, J=1.2 Hz, 3H).

### 2-((3,5-dimethylphenyl)amino)-6-methyl-5-(prop-1-en-2-yl)nicotinonitrile (1c-II)

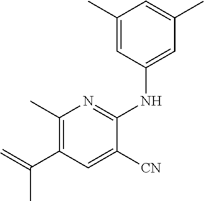 [SMILES: C=C(C)c1cc(C#N)c(Nc2cc(C)cc(C)c2)nc1C]

General procedure A was followed using 3,5-dimethylaniline (125 mL, 1.0 mmol, 1.0 equiv), 2-methyl-1-penten-3-yne 1c-I (106 mL, 1.0 mmol, 1.0 equiv), tert-butylisonitrile (170 mL, 1.5 mmol, 1.5 equiv), Ti(dpm)(NMe₂)₂ (31 mg, 0.1 mmol, 10 mol %), and 2 mL of dry toluene. The second step used malononitrile (132 mg, 2.0 mmol, 2.0 equiv), DBU (76 mg, 0.5 mmol, 0.5 equiv), molecular sieves (200 mg), 2 mL of ethanol and was heated for 2 h. Removal of solvent afforded product as a yellow oil (47 mg, 17%). ¹H NMR (CDCl₃, 500 MHz, 21° C.): δ 7.50 (s, 1H), 7.28 (s, 2H), 6.82 (s, 1H), 6.74 (s, 1H), 5.28-5.22 (m, 1H), 4.91 (dd, J=1.6, 0.8 Hz, 1H), 2.50 (s, 3H), 2.33 (s, 6H), 2.02-2.01 (m, 3H). LRMS (El): calc'd: 277; found: 276. The NMR shows a compound with modest purity, that was used without further purification in the next step.

### 6-Methyl-2-[(3,5-dimethylphenyl)amino]-5-(isopropyl)nicotinonitrile (1c)

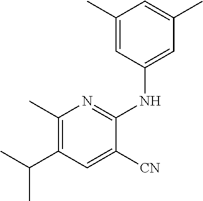 [SMILES: Cc1cc(C)cc(Nc2nc(C)c(C(C)C)cc2C#N)c1]

6-Methyl-2-[(3,5-dimethylphenyl)amino]-5-(prop-1-en-2-1.5 yl)nicotinonitrile 1c-II (80 mg, 0.28 mmol) was dissolved in dry ethanol (6 mL) in a 100 mL Schlenk flask. Palladium on carbon (10%, 100 mg) was added. The flask was flushed with purified dinitrogen, then with dihydrogen gas. The joint was fit with an adaptor for a hydrogen-filled balloon and was stirred at room temperature (25° C.) for 30 min. Purification was accomplished by filtration through neutral alumina, followed by column chromatography (neutral alumina, hexanes:EtOAc 10:1), which afforded the desired compound as a yellow liquid (35%, 28 mg, 0.1 mmol). ¹H NMR (CDCl₃, 500 MHz, 21° C.): 7.59 (s, 1H), 7.29 (s, 2H), 6.77 (s, 1H), 6.73 (s, 1H), 3.06 (hept, J=6.9 Hz, 1H), 2.54 (s, 3H), 2.34 (s, 6H), 1.22 (d, J=6.9 Hz, 6H). ¹³C{¹H} NMR (CDCl₃, 126 MHz, 21° C.): 160.1, 153.4, 139.2, 138.6, 138.1, 132.5, 124.9, 117.7, 117.3, 90.6, 29.8, 28.5, 23.0, 21.6. LRMS (El): calc'd: 279; found: 279.

### 2-((3,5-dimethylphenyl)amino)-5-(p-tolyl)nicotinonitrile (1d)

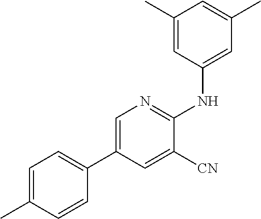

General procedure A was followed using 3,5-dimethylaniline (125 mL, 1.0 mmol, 1.0 equiv), 4-ethynyltoluene (127 mL, 1.0 mmol, 1.0 equiv), tert-butylisonitrile (170 mL, 1.5 mmol, 1.5 equiv), Ti(dpm)(NMe₂)₂ (32 mg, 0.1 mmol, 10 mol %), and dry toluene (2 mL). The second step used malononitrile (132 mg, 2.0 mmol, 2.0 equiv), DBU (76 mg, 0.5 mmol, 0.5 equiv), molecular sieves (200 mg), 2 mL of ethanol and was heated for 2 h. Removal of solvent afforded product as a white solid (31 mg, 10%). M.p.: 112-113° C. ¹H NMR (CDCl₃, 500 MHz, 21° C.): δ 8.62 (d, J=2.5 Hz, 1H), 7.95 (d, J=2.5 Hz, 1H), 7.39 (d, J=8.1 Hz, 2H), 7.28 (d, J=7.9 Hz, 2H), 7.23 (s, 2H), 6.94 (s, 1H), 6.79 (s, 1H), 2.41 (s, 3H), 2.35 (s, 6H). ¹³C{1H} NMR (CDCl₃, 126 MHz, 21° C.): δ 150.79, 139.53, 138.41, 137.99, 133.34, 130.07, 127.53, 126.21, 126.09, 118.88, 116.66, 21.60, 21.27. LRMS (EI): calc'd: 313; found: 312.

### 2-((3,5-dimethylphenyl)amino)-5-(4-methoxyphenyl)nicotinonitrile (1e)

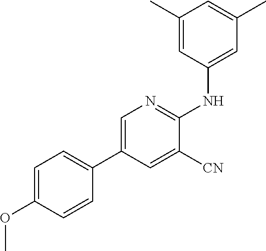 [SMILES: COc1ccc(-c2cnc(Nc3cc(C)cc(C)c3)c(C#N)c2)cc1]

General procedure A was followed using 3,5-dimethylaniline (125 mL, 1.0 mmol, 1.0 equiv), 4-ethynylanisole (130 mL, 1.0 mmol, 1.0 equiv), tert-butylisonitrile (170 mL, 1.5 mmol, 1.5 equiv), Ti(dpm)(NMe₂)₂ (32 mg, 0.1 mmol, 10 mol %), and dry toluene (2 mL). The second step used malononitrile (132 mg, 2.0 mmol, 2.0 equiv), DBU (76 mg, 0.5 mmol, 0.5 equiv), molecular sieves (200 mg), 2 mL of ethanol and was heated for 2 h. Removal of solvent afforded product as a white solid (39 mg, 12%). M.p.: 141-142° C. ¹H NMR (CDCl₃, 500 MHz, 21° C.): δ 8.59 (d, J=2.4 Hz, 1H), 7.92 (d, J=2.9 Hz, 1H), 7.42 (d, J=8.5 Hz, 2H), 7.23 (s, 2H), 7.00 (d, J=8.6 Hz, 2H), 6.92 (s, 1H), 6.79 (s, 1H), 3.86 (s, 3H), 2.34 (s, 6H). ¹³C{1H} NMR (CDCl₃, 126 MHz, 21° C.): δ 159.71, 154.94, 150.59, 139.32, 138.94, 138.46, 128.73, 127.54, 127.37, 126.07, 118.85, 116.70, 114.82, 93.19, 55.55, 21.61. LRMS (El): calc'd: 329; found: 328.

### 5-(4-chlorophenyl)-2-((3,5-dimethylphenyl)amino)nicotinonitrile (1f)

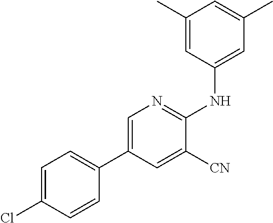 [SMILES: Cc1cc(C)cc(Nc2ncc(-c3ccc(Cl)cc3)cc2C#N)c1]

General procedure A was followed using 3,5-dimethylaniline (125 mL, 1.0 mmol, 1.0 equiv), 1-chloro-4-ethynylbenzene (136 mg, 1.0 mmol, 1.0 equiv), tert-butylisonitrile (170 mL, 1.5 mmol, 1.5 equiv), Ti(dpm)(NMe₂)₂ (32 mg, 0.1 mmol, 10 mol %), and dry toluene (2 mL). The second step used malononitrile (132 mg, 2.0 mmol, 2.0 equiv), DBU (76 mg, 0.5 mmol, 0.5 equiv), molecular sieves (200 mg), 2 mL of ethanol and was heated for 2 h. Removal of solvent afforded product as light yellow crystals (55 mg, 17%). M.p.: 147-148° C. ¹H NMR (CDCl₃, 500 MHz, 21° C.): δ 8.59 (d, J=2.5 Hz, 1H), 7.93 (d, J=2.5 Hz, 1H), 7.47-7.38 (m, 4H), 7.23 (s, 2H), 6.99 (s, 1H), 6.80 (s, 1H), 2.35 (s, 6H). ¹³C{1H} NMR (CDCl₃, 126 MHz, 21° C.): δ 155.42, 150.78, 139.58, 138.95, 138.17, 134.69, 134.20, 129.56, 127.57, 126.32, 126.28, 119.03, 116.40, 93.27, 21.59. LRMS (EI): calc'd: 333; found: 332.

### 5-(3-chlorophenyl)-2-((3,5-dimethylphenyl)amino)-6-methylnicotinonitrile (1g)

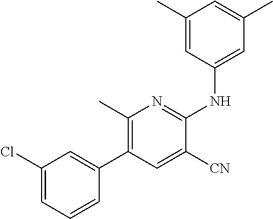 [SMILES: Cc1cc(C)cc(Nc2nc(C)c(-c3cccc(Cl)c3)cc2C#N)c1]

General procedure A was followed using 3,5-dimethylaniline (250 mL, 2.0 mmol, 1.0 equiv), 1-chloro-3-(prop-1-yn-1-yl)benzene (300 mg, 2.0 mmol, 1.0 equiv), tert-butylisonitrile (340 mL, 3.0 mmol, 1.5 equiv), Ti(dpm)(NMe₂)₂ (64 mg, 0.2 mmol, 10 mol %), and 4 mL of dry toluene. The second step used malononitrile (264 mg, 4.0 mmol, 2.0 equiv), DBU (152 mg, 1.0 mmol, 0.5 equiv), molecular sieves (400 mg), 4 mL of ethanol and was heated for 2 h. Removal of solvent afforded product as a white solid (127 mg, 18%). M.p.: 136-137° C. ¹H NMR (CDCl₃, 500 MHz, 21° C.): δ 7.60 (s, 1H), 7.38-7.34 (m, 2H), 7.31 (s, 2H), 7.29-7.27 (m, 1H), 7.19-7.13 (m, 1H), 6.92 (s, 1H), 6.77 (s, 1H), 2.46 (s, 3H), 2.34 (s, 7H). ¹³C{1H} NMR (CDCl₃, 126 MHz, 21° C.): δ 154.56, 142.24, 140.06, 138.80, 138.63, 134.63, 130.03, 129.32, 127.96, 127.48, 126.59, 125.64, 118.27, 116.60, 90.52, 24.19, 21.65.

### 5-(3-chlorophenyl)-2-((3,5-dimethylbhenvflamino)nicotinonitrile (1h)

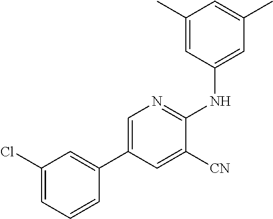 [SMILES: Cc1cc(C)cc(Nc2ncc(-c3cccc(Cl)c3)cc2C#N)c1]

General procedure A was followed using 3,5-dimethylaniline (125 mL, 1.0 mmol, 1.0 equiv), 1-chloro-3-ethynylbenzene (136 mg, 1.0 mmol, 1.0 equiv), tert-butylisonitrile (170 mL, 1.5 mmol, 1.5 equiv), Ti(dpm)(NMe₂)₂ (32 mg, 0.1 mmol, 10 mol %), and 2 mL of dry toluene. The second step used malononitrile (132 mg, 2.0 mmol, 2.0 equiv), DBU (76 mg, 0.5 mmol, 0.5 equiv), molecular sieves (200 mg), 2 mL of ethanol and was heated for 2 h. Removal of solvent afforded product as a white solid (76 mg, 23%). M.p.: 125-126° C. ¹H NMR (CDCl₃, 500 MHz, 21° C.): δ 8.60 (s, 1H), 7.96 (s, 1H), 7.53-7.46 (m, 1H), 7.45-7.32 (m, 3H), 7.23 (s, 2H), 6.99 (s, 1H), 6.81 (s, 1H), 2.35 (s, 6H). ¹³C{1H} NMR (CDCl₃, 126 MHz, 21° C.): δ 155.47, 150.82, 139.62, 138.87, 137.99, 137.94, 130.50, 127.95, 126.37, 126.27, 125.98, 124.35, 118.96, 116.22, 93.18, 21.47. LRMS (EI): calc'd: 333; found: 332.

### 5-(3-((tert-butyldimethylsilyl)oxy)phenyl)-2-((3,5-dimethylphenyl)amino)nicotinonitrile (1i)

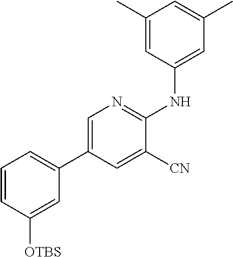 [SMILES: Cc1cc(C)cc(Nc2ncc(-c3cccc(O[Si](C)(C)C(C)(C)C)c3)cc2C#N)c1]

General procedure A was followed using 3,5-dimethylaniline (250 mL, 2.0 mmol, 1.0 equiv), tert-butyl(3-ethynylphenoxy)dimethylsilane (464 mg, 2.0 mmol, 1.0 equiv), tert-butylisonitrile (340 mL, 3.0 mmol, 1.5 equiv), Ti(dpm)(NMe₂)₂ (64 mg, 0.2 mmol, 10 mol %), and 5 mL of dry toluene. The second step used malononitrile (264 mg, 4.0 mmol, 2.0 equiv), DBU (152 mg, 1.0 mmol, 0.5 equiv), molecular sieves (200 mg), 2 mL of ethanol and was heated for 2 h. Removal of solvent afforded product as a white solid (160 mg, 19%). M.p.: 104-105° C. ¹H NMR (CDCl₃, 500 MHz, 21° C.): δ 8.61 (d, J=2.4 Hz, 1H), 7.94 (d, J=2.5 Hz, 1H), 7.32 (t, J=7.9 Hz, 1H), 7.23 (s, 2H), 7.08 (d, J=7.8 Hz, 1H), 6.99-6.94 (m, 2H), 6.86 (dd, J=8.1, 1.7 Hz, 1H), 6.80 (s, 1H), 2.35 (s, 6H), 1.01 (s, 9H), 0.24 (s, 6H). ¹³C{1H} NMR (CDCl₃, 126 MHz, 21° C.): δ 156.54, 155.36, 150.96, 139.74, 138.95, 138.33, 137.63, 130.38, 127.32, 126.20, 119.70, 119.36, 118.99, 118.12, 116.61, 93.17, 25.81, 21.60, 18.37, −4.20.

### 5-phenyl-2-((3,4,5-trimethylphenyl)amino)nicotinonitrile (1j)

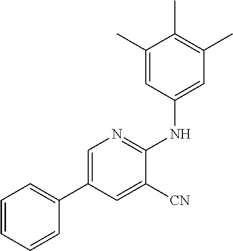 [SMILES: Cc1cc(Nc2ncc(-c3ccccc3)cc2C#N)cc(C)c1C]

General procedure A was followed using 3,4,5-trimethylaniline (140 mL, 1.0 mmol, 1.0 equiv), phenylacetylene (110 mL, 1.0 mmol, 1.0 equiv), tert-butylisonitrile (170 mL, 1.5 mmol, 1.5 equiv), Ti(dpm)(NMe₂)₂ (32 mg, 0.1 mmol, 10 mol %), and 2 mL of dry toluene. The second step used malononitrile (132 mg, 2.0 mmol, 2.0 equiv), DBU (76 mg, 0.5 mmol, 0.5 equiv), molecular sieves (200 mg), 2 mL of ethanol and was heated for 2 h. Removal of solvent afforded product as a white solid (43 mg, 14%). ¹H NMR (CDCl₃, 500 MHz, 21° C.): δ 8.61 (s, 1H), 7.96 (s, 1H), 7.54-7.43 (m, 4H), 7.39 (t, J=7.7 Hz, 1H), 7.23 (s, 2H), 6.91 (s, 1H), 2.32 (s, 6H), 2.17 (s, 3H). ¹³C{1H} NMR (CDCl₃, 126 MHz, 21° C.): δ 155.58, 151.00, 139.62, 137.25, 136.23, 135.18, 131.60, 129.24, 127.85, 127.10, 126.22, 121.06, 116.61, 92.73, 20.83, 15.04. LRMS (EI): calc'd: 313; found: 312.

### 5-(2-chlorophenyl)-2-((3,5-dimethylphenyl)amino)nicotinonitrile (1k)

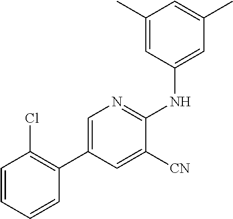 [SMILES: Cc1cc(C)cc(Nc2ncc(-c3ccccc3Cl)cc2C#N)c1]

General procedure A was followed using 3,5-dimethylaniline (250 mL, 1.0 mmol, 1.0 equiv), 1-chloro-3-ethynylbenzene (272 mg, 2.0 mmol, 1.0 equiv), tert-butylisonitrile (340 mL, 3.0 mmol, 1.5 equiv), Ti(dpm)(NMe₂)₂ (64 mg, 0.2 mmol, 10 mol %), and 5 mL of dry toluene. The second step used malononitrile (264 mg, 4.0 mmol, 2.0 equiv), DBU (152 mg, 1.0 mmol, 0.5 equiv), molecular sieves (400 mg), 4 mL of ethanol and was heated for 2 h. Removal of solvent afforded product as a white solid (149 mg, 22%). M.p.: 139-140° C. ¹H NMR (CDCl₃, 500 MHz, 21° C.): δ 8.46 (d, J=1.9 Hz, 1H), 7.99-7.85 (m, 1H), 7.53-7.47 (m, 1H), 7.39-7.28 (m, 3H), 7.24 (s, 2H), 6.99 (s, 1H), 6.81 (s, 1H), 2.35 (s, 6H). ¹³C{1H} NMR (CDCl₃, 126 MHz, 21° C.): δ 155.26, 152.76, 142.17, 138.85, 138.03, 135.24, 134.99, 130.90, 130.32, 129.49, 127.37, 126.24, 119.04, 116.33, 109.53, 92.40, 21.47. LRMS (EI): calc'd: 333; found: 332.

### 2-((3,5-dimethylphenyl)amino)-5-phenyl-6-propylnicotinonitrile (1l)

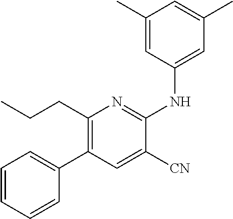 [SMILES: CCCc1nc(Nc2cc(C)cc(C)c2)c(C#N)cc1-c1ccccc1]

General procedure A was followed using 3,5-dimethylaniline (125 mL, 1.0 mmol, 1.0 equiv), 1-phenyl-1-pentyne (160 mL, 1.0 mmol, 1.0 equiv), tert-butylisonitrile (170 mL, 1.5 mmol, 1.5 equiv), Ti(dpm)(NMe₂)₂ (31 mg, 0.1 mmol, 10 mol %), and 2 mL of dry toluene. The second step used malononitrile (132 mg, 2.0 mmol, 2.0 equiv), DBU (76 mg, 0.5 mmol, 0.5 equiv), molecular sieves (200 mg), 2 mL of ethanol and was heated for 2 h. Removal of solvent afforded product as a white powder (113 mg, 34%). M.p.: 126-127° C. ¹H NMR (CDCl₃, 500 MHz, 21° C.): δ 7.60 (s, 1H), 7.43 (t, J=7.2 Hz, 2H), 7.38 (t, J=7.3 Hz, 1H), 7.36 (s, 2H), 7.26 (d, 2H), 6.92 (s, 1H), 6.75 (s, 1H), 2.70 (t, J=7.6 Hz, 2H), 2.34 (s, 6H), 1.79 (h, J=7.4 Hz, 2H), 0.90 (t, J=7.4 Hz, 3H). ¹³C{1H} NMR (CDCl₃, 126 MHz, 21° C.): δ 163.60, 154.38, 142.28, 138.96, 138.61, 138.39, 129.35, 128.67, 128.15, 127.69, 125.10, 117.95, 116.90, 90.13, 37.73, 21.78, 21.59, 14.08. LRMS (EI): calc'd: 341; found: 340.

### 2-((3-bromo-5-methylphenyl)amino)-6-methyl-5-phenylnicotinonitrile (1m)

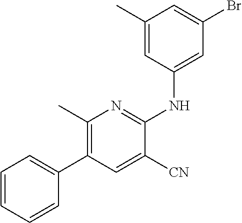 [SMILES: Cc1cc(Br)cc(Nc2nc(C)c(-c3ccccc3)cc2C#N)c1]

The general procedure A was followed using 3-bromo-5-methylaniline (370 mg, 2.0 mmol, 1.0 equiv), 1-phenyl-1-propyne (250 mL, 2.0 mmol, 1.0 equiv), tert-butylisonitrile (340 mL, 3.0 mmol, 1.5 equiv), Ti(dpm)(NMe₂)₂ (62 mg, 0.2 mmol, 10 mol %), and 5 mL of dry toluene. The second step used malononitrile (264 mg, 4.0 mmol, 2.0 equiv), DBU (152 mg, 1.0 mmol, 0.5 equiv), molecular sieves (400 mg), 4 mL of ethanol and was heated for 12 h. Removal of solvent afforded product as a white powder (168 mg, 19%). M.p.: 161-162° C. ¹H NMR (CDCl₃, 500 MHz, 21° C.): δ 7.63 (s, 1H), 7.46 (s, 2H), 7.44 (t, J=7.4 Hz, 2H), 7.38 (t, J=7.3 Hz, 1H), 7.27 (d, J=8.5 Hz, 2H), 6.87 (s, 1H), 2.78 (s, 3H), 2.47 (s, 3H). ¹³C{1H} NMR (CDCl₃, 126 MHz, 21° C.): δ 160.36, 154.07, 142.26, 138.87, 138.18, 137.55, 130.20, 129.20, 128.79, 128.42, 127.86, 121.36, 119.96, 116.73, 90.67, 29.71, 24.25. LRMS (EI): calc'd: 377; found: 378.

### 6-methyl-5-phenyl-2-(m-tolylamino)nicotinonitrile (1n)

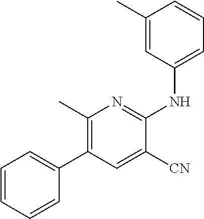 [SMILES: Cc1cccc(Nc2nc(C)c(-c3ccccc3)cc2C#N)c1]

General procedure A was followed using m-toluidine (214 mL, 2.0 mmol, 1.0 equiv), 1-phenyl-1-propyne (250 mL, 2.0 mmol, 1.0 equiv), tert-butylisonitrile (340 mL, 3.0 mmol, 1.5 equiv), Ti(dpm)(NMe₂)₂ (62 mg, 0.2 mmol, 10 mol %), and 5 mL of dry toluene. The second step used malononitrile (264 mg, 4.0 mmol, 2.0 equiv), DBU (152 mg, 1.0 mmol, 0.5 equiv), molecular sieves (400 mg), 4 mL of ethanol and was heated for 2 h. Removal of solvent afforded product as a white powder (155 mg, 29%). M.p.:117-118° C. ¹H NMR (CDCl₃, 500 MHz, 21° C.):δ 7.64 (s, 1H), 7.59 (d, J=8.1 Hz, 1H), 7.49-7.42 (m, 3H), 7.39 (t, J=8.0 Hz, 1H), 7.31-7.23 (m, 4H), 6.94 (s, 1H), 6.93 (s, 1H), 2.49 (s, 3H), 2.39 (s, 3H). ¹³C{1H} NMR (CDCl₃, 126 MHz, 21° C.): 6160.36, 154.29, 142.23, 139.00, 138.95, 138.31, 129.23, 128.95, 128.75, 128.18, 127.78, 124.40, 120.85, 117.36, 116.85, 90.48, 24.28, 21.74. LRMS (EI): calc'd: 299; found: 298.

### 2-((2,5-dimethylphenyl)amino)-6-methyl-5-phenylnicotinonitrile (1o)

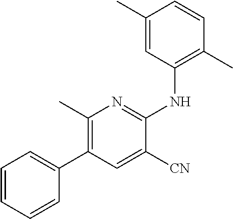 [SMILES: Cc1ccc(C)c(Nc2nc(C)c(-c3ccccc3)cc2C#N)c1]

General procedure A was followed using 2,5-xylidine (249 mL, 2.0 mmol, 1.0 equiv), 1-phenyl-1-propyne (250 mL, 2.0 mmol, 1.0 equiv), tert-butylisonitrile (340 mL, 3.0 mmol, 1.5 equiv), Ti(dpm)(NMe₂)₂ (62 mg, 0.2 mmol, 10 mol %), and 5 mL of dry toluene. The second step used malononitrile (264 mg, 4.0 mmol, 2.0 equiv), DBU (152 mg, 1.0 mmol, 0.5 equiv), molecular sieves (400 mg), 4 mL of ethanol and was heated for 2 h. Removal of solvent afforded product as a white powder (96 mg, 15%). M.p.: 128-129° C. ¹H NMR (CDCl₃, 500 MHz, 21° C.):δ 7.81 (s, 1H), 7.62 (s, 1H), 7.44 (t, J=7.3 Hz, 2H), 7.41-7.35 (m, 1H), 7.30-7.26 (m, 2H), 7.13 (d, J=7.7 Hz, 1H), 6.92 (d, J=8.4 Hz, 1H), 6.79 (s, 1H), 2.43 (s, 3H), 2.36 (s, 3H), 2.31 (s, 3H). ¹³C{1H} NMR (CDCl₃, 126 MHz, 21° C.): δ 160.48, 154.88, 142.24, 138.39, 136.92, 136.36, 130.57, 129.24, 128.73, 127.95, 127.73, 126.91, 125.43, 123.25, 116.89, 90.25, 24.29, 21.44, 17.81. LRMS (EI): calc'd: 313; found: 312.

### 6-methyl-5-phenyl-2-((3,4,5-trimethylphenyl)amino)nicotinonitrile (1p)

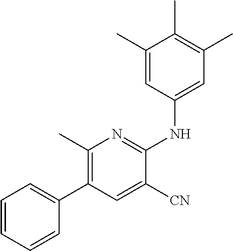 [SMILES: Cc1cc(Nc2nc(C)c(-c3ccccc3)cc2C#N)cc(C)c1C]

General procedure A was followed using 3,4,5-trimethylaniline (280 mL, 2.0 mmol, 1.0 equiv), 1-phenyl-1-propyne (250 mL, 2.0 mmol, 1.0 equiv), tert-butylisonitrile (340 mL, 3.0 mmol, 1.5 equiv), Ti(dpm)(NMe₂)₂ (62 mg, 0.2 mmol, 10 mol %), and 5 mL of dry toluene. The second step used malononitrile (264 mg, 4.0 mmol, 2.0 equiv), DBU (152 mg, 1.0 mmol, 0.5 equiv), molecular sieves (400 mg), 4 mL of ethanol and was heated for 2 h. Removal of solvent afforded product as a white powder (98 mg, 15%). M.p.: 167-177° C. ¹H NMR (CDCl₃, 500 MHz, 21° C.): δ 7.61 (s, 1H), 7.48-7.42 (m, 2H), 7.41-7.36 (m, 1H), 7.34 (s, 2H), 7.29-7.27 (m, 2H), 6.84 (s, 1H), 2.52 (s, 3H), 2.31 (s, 6H), 2.17 (s, 3H). ¹³C{1H} NMR (CDCl₃, 126 MHz, 21° C.): δ 160.38, 154.55, 142.20, 138.44, 137.13, 135.96, 130.65, 129.24, 128.72, 127.79, 127.69, 119.89, 116.99, 90.10, 24.29, 20.99, 15.08. LRMS (EI): calc'd: 327; found: 326.

### 2-((3-isopropylphenyl)amino)-6-methyl-5-phenylnicotinonitrile (1q)

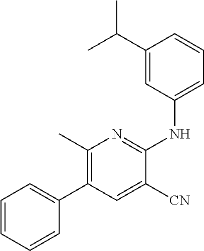 [SMILES: Cc1nc(Nc2cccc(C(C)C)c2)c(C#N)cc1-c1ccccc1]

General procedure A was followed using 3-isopropylaniline (281 mL, 2.0 mmol, 1.0 equiv), 1-phenyl-1-propyne (250 mL, 2.0 mmol, 1.0 equiv), tert-butylisonitrile (340 mL, 3.0 mmol, 1.5 equiv), Ti(dpm)(NMe₂)₂ (62 mg, 0.2 mmol, 10 mol %), and 5 mL of dry toluene. The second step used malononitrile (264 mg, 4.0 mmol, 2.0 equiv), DBU (152 mg, 1.0 mmol, 0.5 equiv), molecular sieves (400 mg), 4 mL of ethanol and was heated for 2 h. Removal of solvent afforded product as a white powder (120 mg, 18%). M.p.: 114-115° C. ¹H NMR (CDCl₃, 500 MHz, 21° C.): δ 7.64 (s, 1H), 7.59-7.51 (m, 2H), 7.44 (t, J=7.3 Hz, 2H), 7.38 (t, J=7.3 Hz, 1H), 7.32-7.27 (m, 3H), 7.26 (s, 1H), 6.99 (s, 1H), 6.97 (s, 1H), 2.93 (hept, J=7.5 Hz, 1H), 2.47 (s, 3H), 1.29 (d, J=6.9 Hz, 6H). ¹³C{1H} NMR (CDCl₃, 126 MHz, 21° C.): δ 154.30, 149.95, 142.24, 139.00, 138.32, 130.13, 129.23, 128.96, 128.76, 128.15, 127.78, 121.93, 118.35, 117.70, 116.88, 90.42, 34.30, 24.28, 24.11. LRMS (EI): calc'd: 327; found: 326.

### 2-((3,5-dichlorophenyl)amino)-6-methyl-5-phenylnicotinonitrile (1r)

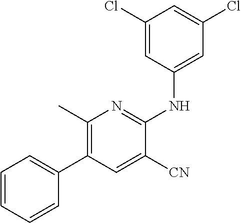 [SMILES: Cc1nc(Nc2cc(Cl)cc(Cl)c2)c(C#N)cc1-c1ccccc1]

General procedure A was followed using 3,5-dichloroaniline (324 mg, 2.0 mmol, 1.0 equiv), 1-phenyl-1-propyne (250 mL, 2.0 mmol, 1.0 equiv), tert-butylisonitrile (340 mL, 3.0 mmol, 1.5 equiv), Ti(dpm)(NMe₂)₂ (62 mg, 0.2 mmol, 10 mol %), and dry toluene (5 mL). The second step used malononitrile (264 mg, 4.0 mmol, 2.0 equiv), DBU (152 mg, 1.0 mmol, 0.5 equiv), molecular sieves (400 mg), 4 mL of ethanol and was heated for 12 h. Removal of solvent afforded product as a white powder (200 mg, 28%). M.p.: 211-212° C. ¹H NMR (CDCl₃, 500 MHz, 21° C.): δ 7.72 (d, J=1.7 Hz, 2H), 7.68 (s, 1H), 7.45 (t, J=7.2 Hz, 2H), 7.40 (t, J=7.3 Hz, 1H), 7.28 (d, J=6.9 Hz, 2H), 7.08 (t, J=1.8 Hz, 1H), 6.98 (s, 1H), 2.52 (s, 3H). ¹³C{1H} NMR (CDCl₃, 126 MHz, 21° C.): δ 160.36, 153.33, 142.43, 140.94, 137.77, 135.26, 129.50, 129.16, 128.88, 128.10, 123.20, 118.09, 116.21, 91.46, 24.16. LRMS (EI): calc'd: 353; found: 352.

### 2-((3,5-dibromophenyl)amino)-6-methyl-5-phenylnicotinonitrile (1s)

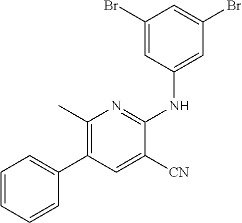 [SMILES: Cc1nc(Nc2cc(Br)cc(Br)c2)c(C#N)cc1-c1ccccc1]

General procedure A was followed using 3,5-dibromoaniline (502 mg, 2.0 mmol, 1.0 equiv), 1-phenyl-1-propyne (250 mL, 2.0 mmol, 1.0 equiv), tert-butylisonitrile (340 mL, 3.0 mmol, 1.5 equiv), Ti(dpm)(NMe₂)₂ (62 mg, 0.2 mmol, 10 mol %), and dry toluene (5 mL). Then, malononitrile (264 mg, 4.0 mmol, 2.0 equiv), DBU (152 mg, 1.0 mmol, 0.5 equiv), molecular sieves (400 mg), 4 mL of ethanol were added, and the solution was heated for 12 h. Removal of solvent afforded product as a white powder (168 mg, 19%). M.p.: 202-203° C. ¹H NMR (CDCl₃, 500 MHz, 21° C.): δ 7.91 (d, J=1.5 Hz, 2H), 7.68 (s, 1H), 7.45 (t, J=7.3 Hz, 2H), 7.40 (t, J=7.3 Hz, 1H), 7.37 (t, J=1.5 Hz, 1H), 7.28 (d, J=6.9 Hz, 2H), 6.98 (s, 1H), 2.51 (s, 3H). ¹³C{1H} NMR (CDCl₃, 126 MHz, 21° C.): δ 160.44, 153.29, 142.32, 141.33, 137.82, 129.51, 129.16, 128.86, 128.50, 128.07, 123.01, 121.30, 116.25, 91.40, 24.22. LRMS (EI): calc'd: 443; found: 443.

### 2-((3-iodophenyl)amino)-6-methyl-5-phenylnicotinonitrile (1t)

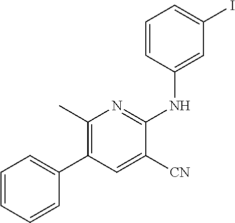 [SMILES: Cc1nc(Nc2cccc(I)c2)c(C#N)cc1-c1ccccc1]

General procedure A was followed using 3-iodoaniline (438 mg, 2.0 mmol, 1.0 equiv), 1-phenyl-1-propyne (250 mL, 2.0 mmol, 1.0 equiv), tert-butylisonitrile (340 mL, 3.0 mmol, 1.5 equiv), Ti(dpm)(NMe₂)₂ (62 mg, 0.2 mmol, 10 mol %), and dry toluene (5 mL). The second step used malononitrile (264 mg, 4.0 mmol, 2.0 equiv), DBU (152 mg, 1.0 mmol, 0.5 equiv), molecular sieves (400 mg), 4 mL of ethanol and was heated for 6 h. Removal of solvent afforded product as a white powder (310 mg, 38%). M.p.: 144-145° C. ¹H NMR (CDCl₃, 500 MHz, 21° C.): δ 8.25-8.21 (m, 1H), 7.66 (s, 1H), 7.62 (ddd, J=8.2, 2.2, 0.8 Hz, 1H), 7.47-7.41 (m, 3H), 7.41-7.37 (m, 1H), 7.30-7.27 (m, 2H), 7.08 (t, J=8.0 Hz, 1H), 6.94 (s, 1H), 2.50 (s, 3H). ¹³C{¹H} NMR (CDCl₃, 126 MHz, 21° C.): δ 160.39, 153.71, 142.27, 140.30, 138.04, 132.29, 130.50, 129.19, 128.90, 128.81, 128.76, 127.94, 119.18, 116.52, 94.29, 90.94, 24.24.

### 2-((3-(((tert-butyldimethylsilyl)oxy)methyl)phenyl)amino)-6-methyl-5-5-phenylnicotinonitrile (1u-I)

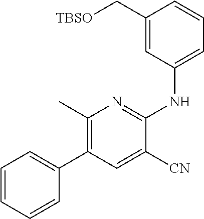 [SMILES: Cc1nc(Nc2cccc(CO[Si](C)(C)C(C)(C)C)c2)c(C#N)cc1-c1ccccc1]

General procedure A was followed using 3-(((tert-butyldimethylsilyl)oxy)methyl)aniline (474 mg, 2.0 mmol, 1.0 equiv), 1-phenyl-1-propyne (250 mL, 2.0 mmol, 1.0 equiv), tert-butylisonitrile (340 mL, 3.0 mmol, 1.5 equiv), Ti(dpm)(NMe₂)₂ (64 mg, 0.2 mmol, 10 mol %), and 4 mL of dry toluene. The second step used malononitrile (264 mg, 4 mmol, 2.0 equiv), DBU (152 mg, 1.0 mmol, 0.5 equiv), molecular sieves (400 mg), 4 mL of ethanol and was heated for 2 h. Removal of solvent afforded product as light yellow oil (384 mg, 45%). ¹H NMR (CDCl₃, 500 MHz, 21° C.): δ 7.64 (s, 2H), 7.61 (d, J=7.6 Hz, 1H), 7.44 (t, J=7.3 Hz, 2H), 7.38 (t, J=7.4 Hz, 1H), 7.32 (t, J=7.8 Hz, 1H), 7.28 (d, J=6.8 Hz, 2H), 7.06 (d, J=7.6 Hz, 1H), 7.00 (s, 1H), 4.77 (s, 2H), 2.47 (s, 3H), 0.96 (s, 9H), 0.13 (s, 6H). The NMR shows a compound with modest purity, that was used without further purification in the next step.

### 2-((3-(1-hydroxyethyl)phenyl)amino)-6-methyl-5-phenylnicotinonitrile (1u)

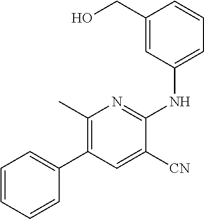 [SMILES: Cc1nc(Nc2cccc(CO)c2)c(C#N)cc1-c1ccccc1]

In a 20 mL glass vial, 2-((3-(((tert-butyldimethylsilyl)oxy)methyl)phenyl)amino)-6-methyl-5-phenylnicotinonitrile (1u-I) (400 mg, 0.93 mmol) was dissolved in 5 mL of THF with a stir bar. To this solution, 3 mL of 1 M TBAF solution (3.0 mmol) in THF was added dropwise under room temperature. The colorless solution became bright orange. After 1 h of stirring, the solvent was removed and crude product was purified by column chromatography (silica hexanes:EtOAc 4:1), which afforded the desired compound as colorless crystals (54%, 163 mg). M.p.: ° C. ¹H NMR (DMSO-d₆, 500 MHz, 21° C.): δ 8.34 (s, 1H), 8.20 (s, 1H), 8.13 (d, J=9.6 Hz, 1H), 8.02 (t, J=7.2 Hz, 3H), 7.95 (t, J=7.4 Hz, 1H), 7.94-7.90 (m, 2H), 7.87 (t, J=7.8 Hz, 1H), 7.62 (d, J=8.6 Hz, 1H), 5.22-5.09 (m, 2H), 3.77 (t, J=6.0 Hz, 1H), 2.94 (s, 3H). ¹³C{1H} NMR (DMSO-d₆, 126 MHz, 21° C.): δ 159.72, 154.42, 143.04, 142.86, 139.61, 138.32, 129.18, 128.55, 127.99, 127.56, 121.52, 119.81, 119.41, 116.35, 90.76, 63.68, 23.28.

### 2-((3-(1-((tert-butyldimethylsilyl)oxy)ethyl)phenyl)amino)-6-methyl-5-phenylnicotinonitrile (1v-I)

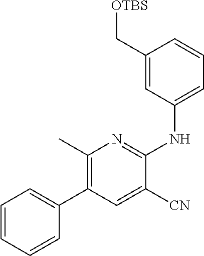 [SMILES: Cc1nc(Nc2cccc(CO[Si](C)(C)C(C)(C)C)c2)c(C#N)cc1-c1ccccc1]

General procedure A was followed using 3-(1-((tert-butyldimethylsilyl)oxy)ethyl)aniline (251 mg, 1.0 mmol, 1.0 equiv), 1-phenyl-1-propyne (125 mL, 1.0 mmol, 1.0 equiv), tert-butylisonitrile (170 mL, 1.5 mmol, 1.5 equiv), Ti(dpm)(NMe₂)₂ (32 mg, 0.1 mmol, 10 mol %), and 2 mL of dry toluene. The second step used malononitrile (132 mg, 2 mmol, 2.0 equiv), DBU (76 mg, 0.5 mmol, 0.5 equiv), molecular sieves (200 mg), 2 mL of ethanol and was heated for 2 h. Removal of solvent afforded product as a white powder (146 mg, 33%). M.p.: 69-70° C. ¹H NMR (CDCl₃, 500 MHz, 21° C.): δ 7.63 (d, J=8.4 Hz, 2H), 7.59 (s, 1H), 7.44 (t, J=7.4 Hz, 2H), 7.38 (t, J=7.4 Hz, 1H), 7.31 (t, J=7.9 Hz, 1H), 7.28 (d, J=7.1 Hz, 2H), 7.08 (d, J=7.6 Hz, 1H), 6.98 (s, 1H), 4.89 (q, 1H), 2.47 (s, 3H), 1.44 (d, J=6.3 Hz, 3H), 0.91 (s, 9H), 0.08 (s, 3H), 0.01 (s, 3H). ¹³C{1H} NMR (CDCl₃, 126 MHz, 21° C.): δ 160.34, 154.29, 148.08, 142.24, 138.86, 138.31, 129.23, 128.86, 128.75, 128.20, 127.78, 120.58, 118.58, 117.27, 116.85, 90.48, 70.92, 27.42, 26.06, 24.29, 18.45, −4.56, −4.66.

### 2-((3-(1-hydroxyethyl)phenyl)amino)-6-methyl-5-phenylnicotinonitrile (1v)

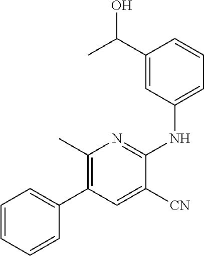 [SMILES: Cc1nc(Nc2cccc(C(C)O)c2)c(C#N)cc1-c1ccccc1]

In a 20 mL glass vial, 2-((3-(1-((tert-butyldimethylsilyl)oxy)ethyl)phenyl)amino)-6-methyl-5-phenylnicotinonitrile (1v-I) (50 mg, 0.11 mmol) was dissolved in 5 mL of THF with a stir bar. To this solution, 1 mL of 1 M TBAF solution (1.0 mmol) in THF was added dropwise under room temperature. The colorless solution became bright orange. After 1 h of stirring, the solvent was removed and crude product was purified by column chromatography (silica hexanes:EtOAc 3:1), which afforded the desired compound as colorless crystals (54%, 20 mg). M.p.: 194-195° C. ¹H NMR (DMSO-d₆, 500 MHz, 21° C.): δ 11.25 (s, 1H), 8.29 (s, 1H), 8.06 (s, 1H), 7.77 (d, J=8.1 Hz, 1H), 7.63 (s, 2H), 7.50-7.43 (m, 4H), 7.37 (ddd, J=8.6, 5.6, 2.3 Hz, 1H), 5 7.24 (t, J=7.8 Hz, 1H), 6.93 (d, J=7.6 Hz, 1H), 4.70 (q, J=6.4 Hz, 1H), 2.42 (s, 3H), 1.34 (d, J=6.4 Hz, 3H). ¹³C{1H} NMR (DMSO-d₆, 126 MHz, 21° C.): δ 170.01, 156.86, 153.28, 148.09, 140.25, 139.06, 138.69, 129.33, 128.37, 127.00, 125.89, 118.60, 117.27, 116.22, 107.79, 68.16, 25.98, 23.47. LRMS (EI): calc'd: 329; found: 328.

### tert-butyl (3-((3-cyano-6-methyl-5-phenylpyridin-2-yl)amino)benzyl)carbamate (1w-I)

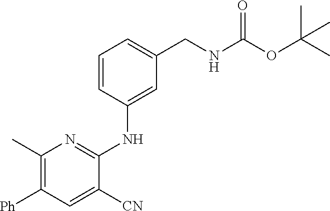 [SMILES: Cc1nc(Nc2cccc(CNC(=O)OC(C)(C)C)c2)c(C#N)cc1-c1ccccc1]

General procedure was followed using 2-chloro-6-methyl-5-phenylnicotinonitrile (200 mg 1.0 equiv), tert-butyl (3-aminobenzyl)carbamate (213 mg, 1.2 equiv), palladium(II) acetate (9 mg, 4 mol %), Xantphos (24 mg, 4.4 mol %), cesium carbonate (1146 mg, 4.0 equiv), and 3 mL of dry toluene. Column condition: 5% of EtOAc in hexanes. Removal of solvent afforded product as a colorless oil (220 mg, 53%). ¹H NMR (CDCl₃, 500 MHz, 21° C.): δ 7.67 (s, 1H), 7.64 (s, 1H), 7.61 (d, J=8.1 Hz, 1H), 7.45 (t, J=7.2 Hz, 2H), 7.39 (t, J=7.4 Hz, 1H), 7.33 (t, J=7.9 Hz, 1H), 7.28 (d, J=6.8 Hz, 2H), 7.02 (d, J=8.4 Hz, 1H), 7.01 (s, 1H), 4.91 (s, 1H), 4.36 (d, J=6.1 Hz, 2H), 2.49 (s, 3H), 1.47 (s, 9H). ¹³C{1H} NMR (CDCl₃, 126 MHz, 21° C.): δ 160.21, 155.90, 154.01, 142.15, 139.87, 139.31, 138.10, 129.22, 129.09, 128.65, 128.30, 127.71, 122.39, 119.10, 116.61, 90.53, 79.55, 53.46, 44.68, 28.45, 24.14.

### 2-((3-(aminomethyl)phenyl)amino)-6-methyl-5-phenylnicotinonitrile (1w)

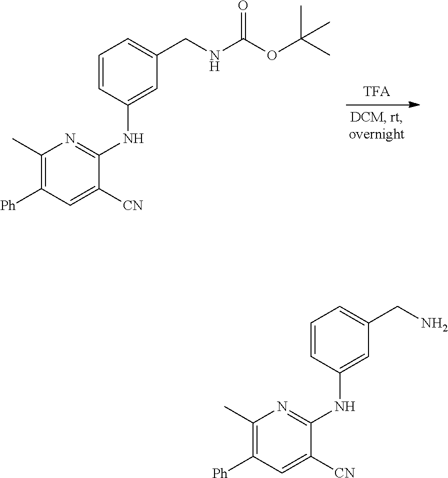

Tert-butyl (3-((3-cyano-6-methyl-5-phenylpyridin-2-yl)amino)benzyl)carbamate was dissolved in 5 mL of DCM in a 20 mL glass vial. To the solution, a 1 mL of TFA (1 mL) was added dropwise and let stir under room temperature overnight. The solution was neutralized by saturated Na₂CO₃ solution. The organic phase was separated, dried over Na2SO₄, and then evaporated. The crude product was purified by column chromatography on silica gel (60% EtOAc in hexanes). Removal of solvent afforded product as a colorless oil (144 mg, 86%). ¹H NMR (CDCl₃, 500 MHz, 21° C.): δ 7.65 (d, J=7.9 Hz, 1H), 7.63 (s, 1H), 7.63 (s, 1H), 7.44 (t, J=7.3 Hz, 2H), 7.38 (t, J=7.4 Hz, 1H), 7.33 (t, J=7.8 Hz, 1H), 7.30-7.25 (m, 2H), 7.10 (s, 1H), 7.05 (d, J=7.6 Hz, 1H), 3.90 (s, 2H), 2.48 (s, 3H). ¹³C{1H} NMR (CDCl₃, 126 MHz, 21° C.): δ 160.17, 154.10, 144.22, 142.16, 139.25, 138.11, 129.13, 129.08, 128.63, 128.17, 127.67, 122.06, 118.71, 118.54, 116.67, 90.46, 46.46, 24.16.

### 2-((3,5-dimethylphenyl)(methyl)amino)-6-methyl-5-phenylnicotinonitrile (2a)

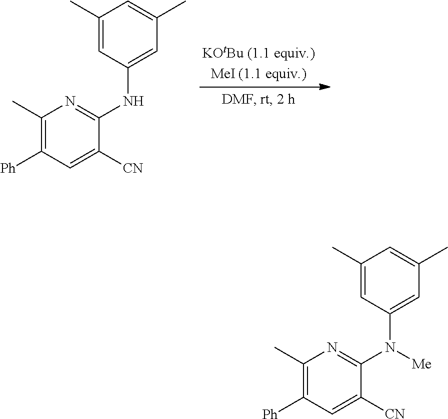

In a 20 mL glass vial, 2-((3,5-dimethylphenyl)amino)-6-methyl-5-phenylnicotinonitrile (80 mg, 1.0 equiv), potassium tert-butoxide (32 mg, 1.1 equiv), and 3 mL of DMF were loaded with a stir bar. The mixture was stirred under room temperature for 10 min before methyl iodide (40 mg, 1.1 equiv) was added dropwise. The mixture was stirred for another 2 h under room temperature. The reaction mixture was poured onto water and extracted with 20 mL of EtOAc. The organic layer was washed with brine, dried with sodium sulfate, and evaporated. The crude product was purified by column chromatography on silica gel (hexane/ethyl acetate) to afford pure product as a white solid (46 mg, 55%). M.p.: 106-107° C. ¹H NMR (CDCl₃, 500 MHz, 21° C.): δ 7.52 (s, 1H), 7.42 (t, J=7.6 Hz, 2H), 7.35 (t, J=7.3 Hz, 1H), 7.28 (d, J=7.9 Hz, 2H), 6.94 (s, 1H), 6.89 (s, 2H), 3.52 (d, J=1.5 Hz, 3H), 2.47 (d, J=1.5 Hz, 3H), 2.33 (s, 6H). ¹³C{1H} NMR (CDCl₃, 126 MHz, 21° C.): δ 159.35, 157.10, 146.51, 144.88, 139.60, 138.52, 129.21, 128.65, 128.56, 127.53, 127.29, 124.04, 116.72, 92.44, 40.73, 24.15, 21.53.

LRMS (EI): calc'd: 327; found: 327.

### N-(3-cyano-6-methyl-5-phenylpyrid in-2-yl)-N-(3,5-dimethylphenyl)acetamide (2b)

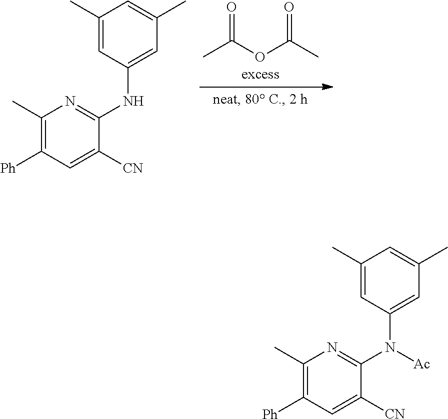

In a 15 mL pressure tube, 2-((3,5-dimethylphenyl)amino)-6-methyl-5-phenylnicotinonitrile (100 mg, 0.32 mmol) and acetic anhydride (2 mL, 21 mmol) were loaded with a stir bar. The tube was sealed and heated in an oil bath at 80° C. for 2 h. After the reaction was cooled to room temperature, the reaction mixture was poured onto water and extracted with 20 mL of EtOAc. The organic layer was washed with brine, dried with sodium sulfate, and evaporated. The crude product was purified by column chromatography on silica gel (hexane/ethyl acetate) to afford pure product as a white solid (78 mg, 69%). M.p.: 161-162° C. ¹H NMR (CDCl₃, 500 MHz, 21° C.): δ 7.83 (s, 1H), 7.51-7.43 (m, 3H), 7.32-7.28 (m, 2H), 7.18 (s, 2H), 7.02 (s, 1H), 2.51 (s, 3H), 2.36 (s, 6H), 2.15 (s, 3H). ¹³C{1H} NMR (CDCl₃, 126 MHz, 21° C.): δ 171.42, 161.32, 154.59, 142.53, 140.61, 139.55, 137.22, 136.30, 130.42, 128.95, 128.60, 126.50, 115.94, 106.44, 24.14, 22.99, 21.38. LRMS (EI): calc'd: 355; found: 355.

### 2-((3,5-dimethylphenyl)amino)-6-methyl-5-phenylnicotinamide (2c)

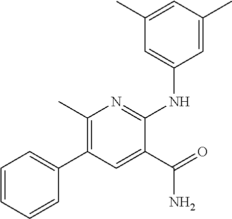 [SMILES: Cc1cc(C)cc(Nc2nc(C)c(-c3ccccc3)cc2C(N)=O)c1]

2-((3,5-dimethylphenyl)amino)-6-methyl-5-phenylnicotinonitrile (110 mg, 0.35 mmol) was placed in a 50 mL round bottom flask with a stir bar and 4 mL of EtOH. This solution was stirred, and tetrabutylammonium hydroxide solution (0.5 mL, 40% in water) was added. An air condenser was attached to the flask, and the solution was heated, in air, until it was actively refluxing. The solution was sampled at 30 min intervals and checked by TLC for completion (watched the starting pyridine disappear). When the starting material no longer appeared by TLC, the reaction was removed from heat and allowed to cool (˜4 h). Once cooled, the solution was neutralized with ammonium chloride until the pH was between 7 and 8. The crude solution was then extracted with DCM several times (5 mL×5). The combined organic fractions were dried over Na₂SO₄, filtered to remove the drying agent, and concentrated by rotary evaporation. This provided the crude product as a sticky orange oil. This oil was washed with water to remove excess NⁿBu₄Cl generated during neutralization. The resulting residue was rinsed with hexanes and dried once more by rotary evaporation. This provided the product as a yellow powder (yield: 27 mg, 23%). From this powder, X-ray quality crystals were grown from a solution of CHCl₃ layered with n-pentane at −20° C. for 3 d. The crystals contain CHCl₃ in the lattice, so this method of purification is undesirable for biological samples due to this solvation. ¹H NMR (CDCl₃, 500 MHz, 21° C.): 10.52 (s, 1H), 7.54 (s, 1H), 7.47-7.40 (m, 4H), 7.39-7.34 (m, 1H), 7.34-7.29 (m, 2H), 6.67 (s, 1H), 5.77 (s (br), 2H), 2.47 (s, 3H), 2.33 (s, 6H). ¹³ C NMR (126 MHz, CDCl₃) δ 170.48, 159.57, 154.20, 140.25, 139.65, 138.34, 137.73, 129.38, 128.63, 127.32, 126.31, 124.07, 118.08, 106.60, 23.88, 21.70. LRMS (EI): calc'd: 331; found: 331.

### 2-((4-bromo-3,5-dimethylphenyl)amino)-6-methyl-5-phenylnicotinonitrile (2d)

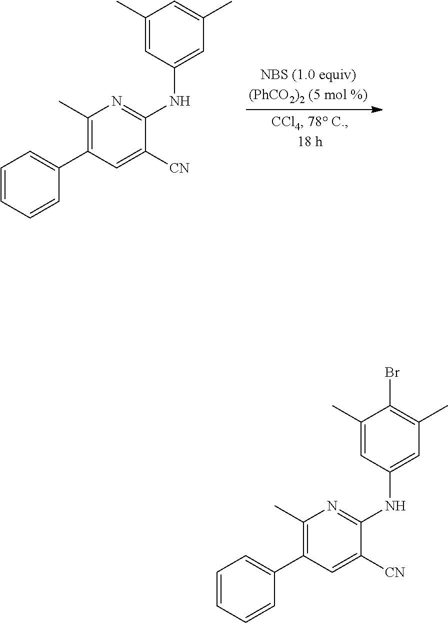 [SMILES: Cc1cc(C)cc(Nc2nc(C)c(-c3ccccc3)cc2C#N)c1 ; Cc1cc(Nc2nc(C)c(-c3ccccc3)cc2C#N)cc(C)c1Br]

In a 50 mL round bottom flask, N-bromosuccinimide (66 mg, 0.37 mmol, 1.0 equiv) and benzoyl peroxide (5.0 mg, 0.05 equiv) was dissolved in 3 mL of 0014 with a stir bar. A solution of 2-((3,5-dimethylphenyl)amino)-6-methyl-5-phenylnicotinonitrile (115 mg, 0.37 mmol, 1.0 equiv) in 3 mL of CCl₄was added. The round bottom flask was fixed with a condenser and heated in an oil bath at 78° C. for 18 h. After the reaction was cooled to room temperature, the reaction mixture was diluted by adding 50 mL of EtOAc, then washed with brine. The organic layer was dried with sodium sulfate and evaporated. The crude product was purified by column chromatography on silica gel (hexane/ethyl acetate) to afford pure product as an off-white solid (124 mg, 86%). ¹H NMR (CDCl₃, 500 MHz, 21° C.): δ 7.63 (s, 1H), 7.46 (s, 2H), 7.44 (t, J=7.5 Hz, 2H), 7.38 (t, J=7.3 Hz, 1H), 7.28 (s, 1H), 6.87 (s, 1H), 2.47 (s, 3H), 2.43 (s, 6H). ¹³C{1H} NMR (CDCl₃, 126 MHz, 21° C.): δ 160.36, 154.07, 142.26, 138.87, 138.18, 137.55, 129.20, 128.78, 128.40, 127.86, 121.36, 119.96, 116.73, 90.67. LRMS (EI): calc'd: 391; found: 392.

### Synthesis of 3-(dimethylamino)-2-phenylacrylaldehyde (3-I)

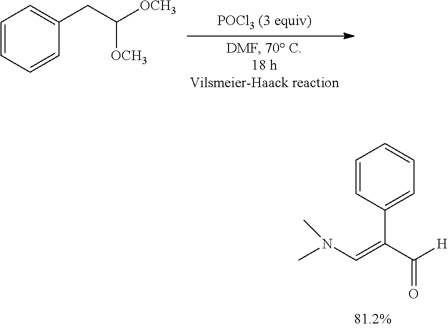

A 250 mL Schlenk flask was loaded with POCl₃ (41.39 g, 0.27 mol, 3 equiv) and a magnetic stir bar. Then, the flask was flushed with dry dinitrogen for 5 min and set in a room temperature water bath. With vigorous stirring, dimethylformamide (24 g, 0.33 mol, 3.67 equiv) was then added dropwise to POCl₃. The mixture was stirred under room temperature for 5 min. After 5 min, phenylacetaldehyde dimethyl acetal (15 g, 0.09 mol, 1 equiv) in 45 mL dimethylformamide was added dropwise for about 5 min. The resulting solution was stirred at 70° C. for 18 h before it was poured into 375 mL ice and neutralized by the addition of anhydrous potassium carbonate till pH is around 7. Then the solution was slowly added sodium hydroxide (50 g, 1.25 mol), and 50 mL of water and heated at 50° C. with stirring for 1 h. The mixture was cooled and extracted with DCM (50 mL×2) and washed with water thoroughly (50 mL×3). The excess solvent was removed in vacuo, resulting a red-brown oil. The crude product was used in the next step without further purification. Further purification can be achieved by cooling concentrated ether solution in a freezer (−30° C.) overnight to afford a red-brown oil (12.79 g, 0.073 mol, 81.2%). 1H NMR (CDCl₃, 500 MHz, 21° C.): δ 9.10 (s, 1H), 7.31-7.35 (m, 2H), 7.22-7.25 (m, 1H), 7.16-7.20 (m, 2H), 6.78 (s, 1H, br), 2.81 (s, 1H, br). ¹³C{1H} NMR (CDCl₃, 126 MHz, 21° C.): 6188.96, 158.60, 133.73, 130.81, 127.45, 126.36, 125.23, 114.86. LRMS (EI): calc'd: 175; found: 175.

### Synthesis of 2-hydroxy-5-phenylnicotinonitrile (3-II)

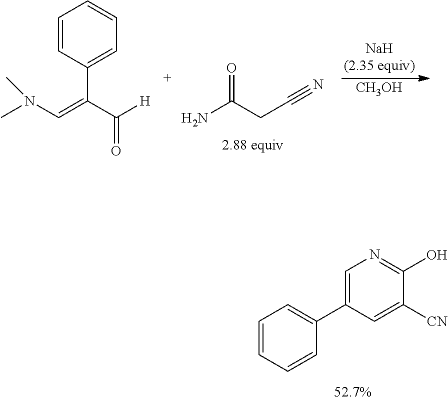

The procedure was adapted from a literature procedure. A 500 mL Schlenk flask was loaded with NaH (3.22 g, 0.134 mol, 2.35 equiv) and a magnetic stir bar before 130 mL of MeOH slowly was added. After 10 min of stirring at room temperature, cyanoacetamide (13.78 g, 0.164 mol, 2.88 equiv), and 3-(dimethylamino)-2-phenylprop-2-enal c-I (10 g, 0.0571 mol, 1.0 equiv) was added. The mixture was stirred at room temperature for 1.5 h and then refluxed overnight. After cooling to room temperature, 100 mL of water was added, and the mixture was acidified with 1 M HCl solution. While adding acid, a large amount of yellow solid precipitated out. The solid was filtered and washed with water (20 mL×3), methanol (5 mL×3), ether (5 mL×3) and hexane (5 mL×3) to afford a light-yellow product. (5.91 g, 0.03 mol, 52.7%). M.p.: 226-227° C. ¹H NMR (CDCl₃, 500 MHz, 21° C.): δ 13.61 (s, 1H, br), 8.23 (s, 1H), 7.99 (s, 1H), 7.45-7.51 (m, 2H), 7.38-7.45 (m, 3H). ¹³C{1H} NMR (CDCl₃, 126 MHz, 21° C.): 6162.11, 147.96, 137.47, 134.04, 129.55, 128.54, 125.88, 121.76, 115.21, 105.35. LRMS (EI): calc'd: 196; found: 196.

### Synthesis of 2-bromo-5-phenylnicotinonitrile (3-III)

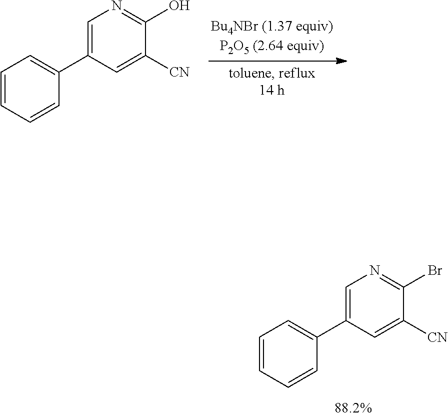 [SMILES: N#Cc1cc(-c2ccccc2)cnc1O ; N#Cc1cc(-c2ccccc2)cnc1Br]

A 250 mL Schlenk flask was charged with 2-hydroxy-5-phenylnicotinonitrile c-II (2.14 g, 10.9 mmol), Bu₄NBr (4.8 g, 14.9 mmol), P₂O₅ (4.1 g, 28.8 mmol), toluene (110 mL), and a magnetic stir bar. The mixture was heated for 14 h under reflux. Then, the toluene layer was decanted and washed with 30 mL of saturated NaHCO₃ solution and then 50 mL of water. 50 mL of water and powered NaHCO₃ was added till no gas was evolved. The mixture was extracted with 250 mL DCM, washed with 2×50 mL of brine, and washed with 30 mL of water. The organic layers were combined and dried with MgSO₄. Removal of the solvent in vacuo afforded a light-yellow power. Further purification can be achieved by chromatography (1:10 EtOAc:hexane). (2.48 g, 9.61 mmol, 88.2%). M.p.: 139-140° C. ¹H NMR (CDCl₃, 500 MHz, 21° C.): δ 8.76 (s, 1H), 8.10 (s, 1H), 7.48-7.57 (m, 5H). ¹³C{1H} NMR (CDCl₃, 126 MHz, 21° C.): 6151.33, 142.07, 140.50, 136.33, 134.41, 129.76, 129.73, 127.15, 115.90, 114.29, 24.99. LRMS (EI): calc'd: 258; found: 257.

### General Procedure B: Synthesis of 2-amino-5-phenylnicotinonitrile Derivatives via Buchwald-Hartwig Coupling

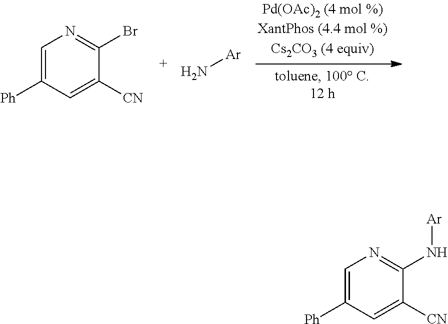

In a glove box, palladium(II) acetate (9.0 mg, 0.04 mmol, 4 mol %) and Xantphos (25.4 mg, 0.044 mmol, 4.4 mol %) were dissolved in 2 mL of dry toluene in a 35 mL pressure tube. The mixture was stirred for 2 min before 2-bromo-5-phenylnicotinonitrile (259 mg, 1.0 mmol, 1.0 equiv), aryl amine (1.2 mmol, 1.2 equiv), and cesium carbonate (1312 mg, 4.0 mmol, 4.0 equiv) were added to the pressure tube. Then, another 3 mL of dry toluene were added. The reaction was then removed from the glovebox and heated in an oil bath at 110° C. for 14 h. After the reaction was cooled to room temperature, the reaction mixture was filtered through Celite and rinsed with ethyl acetate. The filtrate was evaporated, and the crude product was purified by column chromatography on silica gel (hexane/ethyl acetate).

### 2-((4,6-dimethyl-1,3,5-triazin-2-yl)amino)-5-phenylnicotinonitrile (3a)

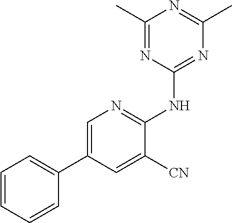 [SMILES: Cc1nc(C)nc(Nc2ncc(-c3ccccc3)cc2C#N)n1]

General procedure B was followed using 2-bromo-5-phenylnicotinonitrile (300 mg, 1.16 mmol, 1.0 equiv), 4,6-dimethyl-1,3,5-triazin-2-amine (172 mg, 1.2 equiv), palladium(II) acetate (10.4 mg, 4 mol %), xantphos (29.5 mg, 4.4 mol %), cesium carbonate (1520 mg, 4.0 equiv), and 5 mL of dry toluene. Removal of solvent afforded product as an off-white powder (42 mg, 12%). M.p.: 225-226° C. ¹H NMR (CDCl₃, 500 MHz, 21° C.): 9.36 (br s, 1H), 8.97 (d, J=2.3 Hz, 1H), 8.22 (d, J=2.3 Hz, 1H), 7.59 (d, J=7.4 Hz, 2H), 7.53 (t, J=7.5 Hz, 2H), 7.47 (t, J=7.3 Hz, 1H), 2.57 (s, 6H). ¹³C{1H} NMR (CDCl₃, 126 MHz, 21° C.): 176.94, 163.43, 150.75, 150.34, 140.75, 135.21, 133.29, 129.46, 128.93, 126.85, 115.77, 103.84, 25.50. LRMS (EI): calc'd: 302; found: 301.

### 2-((4,6-dimethylpyrimidin-2-yl)amino)-5-phenylnicotinonitrile (3b)

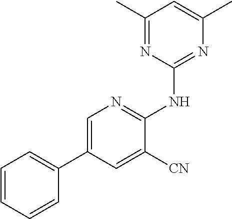 [SMILES: Cc1cc(C)nc(Nc2ncc(-c3ccccc3)cc2C#N)n1]

General procedure B was followed using 2-bromo-5-phenylnicotinonitrile (200 mg, 1.0 equiv), 4,6-dimethylpyrimidin-2-amine (114 mg, 1.2 equiv), palladium(II) acetate (7 mg, 4 mol %), xantphos (19.6 mg, 4.4 mol %), cesium carbonate (1013 mg, 4.0 equiv), and 4 mL of dry toluene. Removal of solvent afforded product as an off-white powder (207 mg, 69%). M.p.: 215-216° C. ¹H NMR (CDCl₃, 500 MHz, 21° C.): δ 8.82 (d, J=2.4 Hz, 1H), 8.25 (s, 1H), 8.13 (d, J=2.4 Hz, 1H), 7.56 (d, J=7.5 Hz, 2H), 7.50 (t, J=7.6 Hz, 2H), 7.43 (t, J=7.3 Hz, 1H), 6.69 (s, 1H), 2.44 (s, 6H). ¹³C{1H} NMR (CDCl₃, 126 MHz, 21° C.): δ 168.18, 157.97, 152.43, 150.58, 140.78, 135.86, 131.52, 129.47, 128.61, 126.84, 116.40, 114.81, 101.06, 23.99. LRMS (EI): calc'd: 301; found: 300.

### 2-((2,6-dimethylpyrimidin-4-yl)amino)-5-phenylnicotinonitrile (3c)

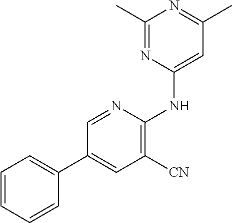 [SMILES: Cc1cc(Nc2ncc(-c3ccccc3)cc2C#N)nc(C)n1]

General procedure B was followed using 2-bromo-5-phenylnicotinonitrile (300 mg, 1.16 mmol, 1.0 equiv), 2,6-dimethylpyrimidin-4-amine (170 mg, 1.2 equiv), palladium(II) acetate (10.4 mg, 4 mol %), xantphos (29.5 mg, 4.4 mol %), cesium carbonate (1520 mg, 4.0 equiv), and 5 mL of dry toluene. Removal of solvent afforded product as an off-white powder (74 mg, 21%). M.p.: 154-155° C. ¹H NMR (CDCl₃, 500 MHz, 21° C.): 8.77 (d, J=2.3 Hz, 1H), 8.11 (s, 1H), 8.09 (d, J=2.5 Hz, 1H), 7.86 (s, 1H), 7.41-7.57 (m, 5H), 2.61 (s, 3H), 2.53 (s, 3H). ¹³C{1H} NMR (CDCl₃, 126 MHz, 21° C.): 151.33, 142.07, 140.50, 136.34, 134.42, 129.76, 129.73, 129.51, 128.70, 127.84, 127.15, 126.89, 119.25, 115.91, 114.30, 21.49. LRMS (EI): calc'd: 301; found: 300.

### 2-((4, 6-dimethylpyridin-2-yl)amino)-5-phenylnicotinonitrile (3d)

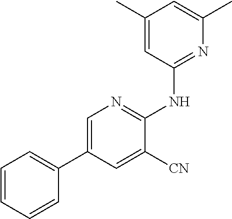 [SMILES: Cc1cc(C)nc(Nc2ncc(-c3ccccc3)cc2C#N)c1]

General procedure B was followed using 2-bromo-5-phenylnicotinonitrile (160 mg, 0.62 mmol, 1.0 equiv), 4,6-dimethylpyridin-2-amine (91 mg, 1.2 equiv), palladium(II) acetate (5.5 mg, 4 mol %), xantphos (15.7 mg, 4.4 mol %), cesium carbonate (810 mg, 4.0 equiv), and 4 mL of dry toluene. The compound was purified by column chromatography on silica gel using 40% EtOAc in hexanes. Removal of solvent afforded product as an off-white powder (93 mg, 50%). M.p.: 112-113° C. ¹H NMR (CDCl₃, 500 MHz, 21° C.): δ 8.70 (d, J=2.4 Hz, 1H), 8.03 (d, J=2.4 Hz, 1H), 8.02 (s, 1H), 7.76 (s, 1H), 7.56-7.46 (m, 4H), 7.41 (t, J=7.2 Hz, 1H), 6.72 (s, 1H), 2.45 (s, 3H), 2.37 (s, 3H). ¹³C{1H} NMR (CDCl₃, 126 MHz, 21° C.): δ 156.85, 153.80, 151.44, 150.41, 149.74, 140.08, 136.05, 129.44, 128.48, 128.28, 126.51, 119.63, 116.03, 110.74, 94.59, 24.06, 21.59. LRMS (EI): calc'd: 300; found: 299.

### 2-((6-methylpyrazin-2-yl)amino)-5-phenylnicotinonitrile (3e)

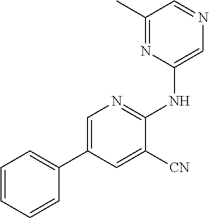 [SMILES: Cc1cncc(Nc2ncc(-c3ccccc3)cc2C#N)n1]

General procedure B was followed using 2-bromo-5-phenylnicotinonitrile (200 mg, 1.0 equiv), 6-methylpyrazin-2-amine (101 mg, 1.2 equiv), palladium(II) acetate (7 mg, 4 mol %), xantphos (19.6 mg, 4.4 mol %), cesium carbonate (1013 mg, 4.0 equiv), and 4 mL of dry toluene. Removal of solvent afforded product as a white powder (77 mg, 35%). M.p.: 160-161° C. 1 H NMR (CDCl₃, 500 MHz, 21° C.): δ 9.59 (s, 1H), 8.74 (d, J=2.5 Hz, 1H), 8.18 (s, 1H), 8.08 (d, J=2.5 Hz, 1H), 7.72 (s, 1H), 7.54 (d, J=7.0 Hz, 2H), 7.50 (t, J=7.6 Hz, 2H), 7.45-7.41 (m, 1H), 2.51 (s, 3H). ¹³C{1H} NMR (CDCl₃, 126 MHz, 21° C.): δ 152.87, 152.09, 150.51, 148.05, 140.08, 138.13, 135.66, 132.55, 129.74, 129.53, 128.60, 126.65, 115.69, 94.97, 21.32. LRMS (EI): calc'd: 287; found: 286.

### 2-((6-methylpyridin-2-yl)amino)-5-phenylnicotinonitrile (3f)

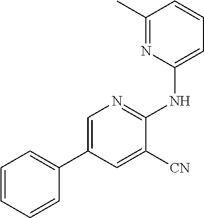 [SMILES: Cc1cccc(Nc2ncc(-c3ccccc3)cc2C#N)n1]

General procedure B was followed using 2-bromo-5-phenylnicotinonitrile (259 mg, 1.0 equiv), 6-methylpyridin-2-amine (130 mg, 1.2 equiv), palladium(II) acetate (9 mg, 4 mol %), xantphos (25.5 mg, 4.4 mol %), cesium carbonate (1312 mg, 4.0 equiv), and 4 mL of dry toluene. Removal of solvent afforded product as a yellow powder (180 mg, 63%). M.p.: 145-146° C. ¹H NMR (CDCl₃, 500 MHz, 21° C.): δ 8.68 (dd, J=2.3, 0.9 Hz, 1H), 8.20 (d, J=8.3 Hz, 1H), 8.04 (dd, J=2.4, 1.0 Hz, 1H), 7.81 (s, 1H), 7.61 (t, J=7.7 Hz, 1H), 7.56-7.45 (m, 4H), 7.41 (t, J=7.6 Hz, 1H), 6.87 (d, J=7.5 Hz, 1H), 2.49 (s, 3H). ¹³C{¹H} NMR (CDCl₃, 126 MHz, 21° C.): δ 157.32, 153.75, 151.36, 150.39, 140.05, 138.43, 135.99, 129.45, 128.63, 128.31, 126.52, 118.31, 116.00, 110.14, 94.62, 24.26. LRMS (EI): calc'd: 286; found: 285.

### 2-((2-methylpyridin-4-yl)amino)-5-phenylnicotinonitrile (3g)

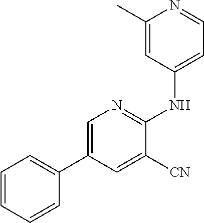 [SMILES: Cc1cc(Nc2ncc(-c3ccccc3)cc2C#N)ccn1]

General procedure B was followed using 2-bromo-5-phenylnicotinonitrile (147 mg, 1.0 equiv), 2-methylpyridin-4-amine (74 mg, 1.2 equiv), palladium(II) acetate (5 mg, 4 mol %), xantphos (15 mg, 4.4 mol %), cesium carbonate (744 mg, 4.0 equiv), and 3 mL of dry toluene. Removal of solvent afforded product as white crystals (110 mg, 68%). M.p.: 133-134° C. ¹H NMR (CDCl₃, 500 MHz, 21° C.): 8.74 (d, J=2.4 Hz, 1H), 8.42 (d, J=6.1 Hz, 1H), 8.06 (d, J=2.4 Hz, 1H), 7.55-7.46 (m, 6H), 7.43 (t, J=7.0 Hz, 1H), 7.16 (s, 1H), 2.58 (s, 3H). ¹³C{1H} NMR (CDCl₃, 126 MHz, 21° C.): δ 159.63, 154.02, 150.55, 150.10, 146.21, 139.89, 135.69, 129.54, 129.45, 128.59, 126.62, 115.93, 112.64, 110.93, 95.02, 24.83. LRMS (EI): calc'd: 286; found: 285.

### 2-((5-methylpyridin-3-yl)amino)-5-phenylnicotinonitrile (3h)

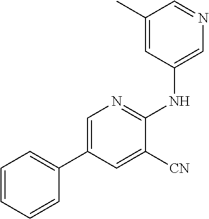 [SMILES: Cc1cncc(Nc2ncc(-c3ccccc3)cc2C#N)c1]

General procedure B was followed using 2-bromo-5-phenylnicotinonitrile (147 mg, 1.0 equiv), 5-methylpyridin-3-amine (74 mg, 1.2 equiv), palladium(II) acetate (5 mg, 4 mol %), xantphos (15 mg, 4.4 mol %), cesium carbonate (744 mg, 4.0 equiv), and 3 mL of dry toluene. Removal of solvent afforded product as white crystals (110 mg, 68%). M.p.: 180-181° C. ¹H NMR (CDCl₃, 500 MHz, 21° C.): 8.64 (d, J=2.4 Hz, 2H), 8.21 (s, 1H), 8.02 (d, J=2.5 Hz, 1H), 7.95 (s, 1H), 7.56-7.44 (m, 4H), 7.40 (t, J=7.7 Hz, 1H), 7.10 (s, 1H), 2.39 (s, 3H). ¹³C{1H} NMR (CDCl₃, 126 MHz, 21° C.): δ ¹³C NMR (126 MHz, CDCl₃) δ 154.76, 150.73, 145.65, 139.94, 139.80, 135.92, 135.11, 133.48, 129.44, 128.56, 128.38, 128.31, 126.46, 116.24, 93.84, 18.63. LRMS (EI): calc'd: 286; found: 285.

### 2-((4-methylpyridin-2-yl)amino)-5-phenylnicotinonitrile (3i)

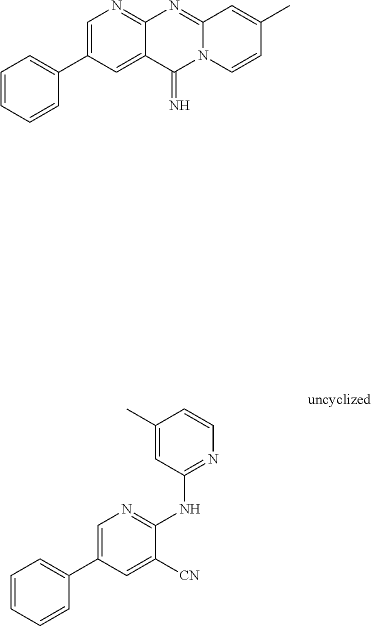 [SMILES: Cc1ccn2c(=N)c3cc(-c4ccccc4)cnc3nc2c1 ; Cc1ccnc(Nc2ncc(-c3ccccc3)cc2C#N)c1]

General procedure B was followed using 2-bromo-5-phenylnicotinonitrile (259 mg, 1.0 equiv), 4-methylpyridin-2-amine (9 mg, 1.2 equiv), palladium(II) acetate (5 mg, 4 mol %), xantphos (25.5 mg, 4.4 mol %), cesium carbonate (1312 mg, 4.0 equiv), and 5 mL of dry toluene. Removal of solvent afforded product as an orange powder (109 mg, 42%). M.p.: 149-150° C. ¹H NMR (CDCl₃, 500 MHz, 21° C.): δ 9.12 (s, 1H), 8.99 (d, J=7.0 Hz, 1H), 8.31 (s, 1H), 7.66 (d, J=7.8 Hz, 2H), 7.51 (t, J=7.6 Hz, 3H), 7.42 (t, J=7.1 Hz, 1H), 7.22 (s, 1H), 6.64 (d, J=7.5 Hz, 1H), 2.40 (s, 3H). ¹³C{1H} NMR (CDCl₃, 126 MHz, 21° C.): δ 154.66, 154.27, 150.45, 147.79, 137.17, 132.38, 131.13, 129.43, 129.37, 128.24, 127.31, 127.06, 126.50, 124.17, 115.35, 109.66, 21.54. ¹H NMR (500 MHz, DMSO-d₆, 21° C.) δ 10.03 (s, 1H), 9.12 (d, J=2.6 Hz, 1H), 9.07 (d, J=2.6 Hz, 1H), 8.97 (d, J=7.5 Hz, 1H), 7.90 (d, J=7.2 Hz, 2H), 7.54 (t, J=7.7 Hz, 2H), 7.43 (t, J=7.4 Hz, 1H), 7.14 (s, 1H), 6.82 (dd, J=7.5, 2.0 Hz, 1H), 2.37 (s, 3H). uncyclized form: ¹H NMR (CDCl₃, 500 MHz, 21° C.): δ 8.71 (d, J=2.4 Hz, 1H), 8.22 (s, 1H), 8.17 (d, J=5.1 Hz, 1H), 8.04 (d, J=2.6 Hz, 1H), 7.81 (s, 1H), 7.65 (s, 2H), 7.47 (d, J=7.9 Hz, 2H), 7.40 (d, J=7.6 Hz, 1H), 6.84 (d, J=4.2 Hz, 1H), 2.41 (s, 3H). LRMS (EI): calc'd: 286; found: 285.

### General Procedure C: Synthesis of 2-amino-5-phenylnicotinonitrile Derivatives via Buchwald-Hartwig Coupling

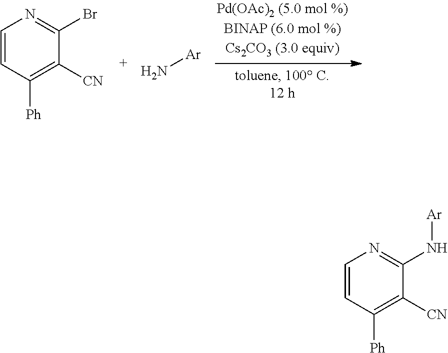

In a glove box, palladium(II) acetate (11.2 mg, 0.05 mmol, 5 mol %) and BINAP (37.7 mg, 0.06 mmol, 6.0 mol %) were dissolved in 2 mL of dry toluene in a 35 mL pressure tube. The mixture was stirred for 2 min before 2-chloro-4-phenylnicotinonitrile (214 mg, 1.0 mmol, 1.0 equiv), aryl amine (1.2 mmol, 1.2 equiv), and cesium carbonate (984 mg, 3.0 mmol, 3.0 equiv) were added to the pressure tube. Then, another 3 mL of dry toluene were added. The reaction was then removed from the glovebox and heated in an oil bath at 110° C. for 14 h. After the reaction was cooled to room temperature, the reaction mixture was filtered through Celite and rinsed with ethyl acetate. The filtrate was evaporated, and the crude product was purified by column chromatography on silica gel (hexane/ethyl acetate).

### 2-((3,5-dimethylphenyl)amino)-4-phenylnicotinonitrile (4a)

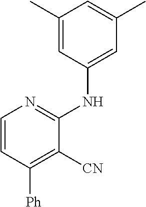 [SMILES: Cc1cc(C)cc(Nc2nccc(-c3ccccc3)c2C#N)c1]

General procedure C was followed using 2-chloro-4-phenylnicotinonitrile (100 mg, 1.0 equiv), dimethyl aniline (73 mL, 1.2 equiv), palladium(II) acetate (5.2 mg, 5 mol %), BINAP (17.6 mg, 6 mol %), cesium carbonate (460 mg, 3.0 equiv), and 3 mL of dry toluene. Removal of solvent afforded product as white crystals (133 mg, 68%). M.p.: 143-144° C. ¹H NMR (CDCl₃, 500 MHz, 21° C.): δ 8.37 (d, J=4.9 Hz, 1H), 7.61 (d, J=7.3 Hz, 2H), 7.56-7.48 (m, 3H), 7.24 (s, 2H), 7.12 (s, 1H), 6.83 (d, J=5.1 Hz, 1H), 6.80 (s, 1H), 2.35 (s, 6H). ¹³C{1H} NMR (CDCl₃, 126 MHz, 21° C.): δ 157.40, 154.90, 151.82, 138.89, 138.49, 136.69, 130.08, 129.11, 128.34, 126.16, 119.20, 116.83, 114.64, 91.73, 21.60. LRMS (EI): calc'd: 299; found: 298.

### 4-phenyl-2-(pyridin-2-ylamino)nicotinonitrile (4b)

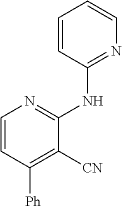 [SMILES: N#Cc1c(-c2ccccc2)ccnc1Nc1ccccn1]

General procedure C was followed using 2-chloro-4-phenylnicotinonitrile (100 mg, 1.0 equiv), 2-aminopyridine (53 mg, 1.2 equiv), palladium(II) acetate (5.2 mg, 5 mol %), BINAP (17.6 mg, 6 mol %), cesium carbonate (460 mg, 3.0 equiv), and 3 mL of dry toluene. The compound was purified by column chromatography on silica gel using pure hexanes. Removal of solvent afforded product as white crystals (120 mg, 94%). M.p.: 150-151° C. ¹H NMR (CDCl₃, 500 MHz, 21° C.): δ 8.50-8.38 (m, 2H), 8.32 (s, 1H), 8.02 (s, 1H), 7.73 (t, J=7.8 Hz, 1H), 7.65-7.57 (m, 2H), 7.52 (s, 3H), 7.05-6.99 (m, 1H), 6.95 (t, J=4.3 Hz, 1H). ¹³C{¹H} NMR (CDCl₃, 126 MHz, 21° C.): δ 155.86, 155.14, 152.30, 151.25, 148.24, 138.11, 136.41, 130.22, 129.16, 128.43, 118.77, 116.04, 115.93, 113.55, 93.24.

### 2-((4-methylpyridin-2-yl)amino)-4-phenylnicotinonitrile (4c)

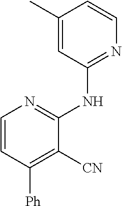 [SMILES: Cc1ccnc(Nc2nccc(-c3ccccc3)c2C#N)c1]

General procedure C was followed using 2-chloro-4-phenylnicotinonitrile (100 mg, 1.0 equiv), 4-methylpyridin-2-amine (61 mg, 1.2 equiv), palladium(II) acetate (5.2 mg, 5 mol %), BINAP (17.6 mg, 6 mol %), cesium carbonate (460 mg, 3.0 equiv), and 3 mL of dry toluene. The compound was purified by column chromatography on silica gel using 20% EtOAc in hexanes. Removal of solvent afforded product as white crystals (97 mg, 73%). M.p.: 123-124° C. ¹H NMR (CDCl₃, 500 MHz, 21° C.): δ 8.47 (d, J=5.2 Hz, 1H), 8.25 (s, 1H), 8.19 (d, J=5.0 Hz, 1H), 7.99 (s, 1H), 7.67-7.59 (m, 2H), 7.58-7.49 (m, 3H), 6.95 (d, J=5.2 Hz, 1H), 6.89-6.82 (m, 1H), 2.43 (s, 3H). ¹³C{¹H} NMR (CDCl₃, 126 MHz, 21° C.): δ. 13C NMR (126 MHz, cdcl3) δ 156.35, 155.92, 155.14, 152.34, 151.27, 149.54, 147.80, 136.44, 130.20, 129.15, 128.43, 120.08, 116.07, 115.80, 113.96, 21.73.

### 2-((4-(((tert-butyldimethylsilyl)oxy)methyl)pyridin-2-yl)amino)-4-phenylnicotinonitrile (4e-I)

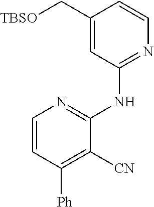 [SMILES: CC(C)(C)[Si](C)(C)OCc1ccnc(Nc2nccc(-c3ccccc3)c2C#N)c1]

General procedure C was followed using 2-chloro-4-phenylnicotinonitrile (200 mg, 1.0 equiv), 4-(((tert-butyldimethylsilyl)oxy)methyl)pyridin-2-amine (267 mg, 1.2 equiv), palladium(II) acetate (10.4 mg, 5 mol %), BINAP (35.2 mg, 6 mol %), cesium carbonate (920 mg, 3.0 equiv), and 6 mL of dry toluene. The compound was purified by column chromatography on silica gel using 20% EtOAc in hexanes. Removal of solvent afforded product as white crystals (286 mg, 74%). M.p.: 102-103° C. ¹H NMR (CDCl₃, 500 MHz, 21° C.): δ 8.49 (s, 1H), 8.42 (d, J=5.2 Hz, 1H), 8.24 (d, J=5.1 Hz, 1H), 8.05 (s, 1H), 7.65-7.58 (m, 2H), 7.57-7.49 (m, 3H), 6.96 (d, J=5.4 Hz, 1H), 6.94 (d, J=5.2 Hz, 1H), 4.81 (s, 2H), 1.00 (s, 9H), 0.15 (s, 6H). ¹³C{¹H} NMR (CDCl₃, 126 MHz, 21° C.): δ 155.88, 155.08, 153.16, 152.49, 151.22, 147.86, 136.47, 130.18, 129.14, 128.43, 116.08, 115.87, 115.81, 110.12, 93.18, 63.92, 26.02, 18.51, −5.19.

### 2-((4-(hydroxymethyl)pyridin-2-yl)amino)-4-phenylnicotinonitrile (4e)

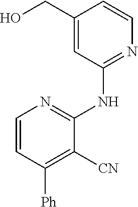 [SMILES: N#Cc1c(-c2ccccc2)ccnc1Nc1cc(CO)ccn1]

In a 20 mL glass vial, 2-((4-(((tert-butyldimethylsilyl)oxy)methyl)pyridin-2-yl)amino)-4-phenylnicotinonitrile 4d-I (85 mg, 1.0 equiv) was dissolved in 5 mL of THF with a stir bar. To this solution, 3 mL of 1 M TBAF solution in THF was added dropwise under room temperature. The colorless solution became bright orange. After 4 h of stirring, the solvent was removed, and the crude product was purified by column chromatography (silica gel, gradient hexanes:EtOAc 1:1 to pure EtOAc), which afforded the desired compound as colorless crystals (75%, 46 mg). M.p.: 151-152° C. ¹H NMR (CDCl₃, 500 MHz, 21° C.): δ 8.43 (d, J=5.2 Hz, 1H), 8.41 (s, 1H), 8.27 (d, J=5.1 Hz, 1H), 8.04 (s, 1H), 7.65-7.57 (m, 2H), 7.57-7.49 (m, 3H), 7.02 (d, J=5.7 Hz, 1H), 6.95 (d, J=5.2 Hz, 1H), 4.79 (s, 2H), 2.37 (s, 1H). ¹³C{¹H} NMR (CDCl₃, 126 MHz, 21° C.): δ 155.81, 155.16, 152.56, 152.37, 151.26, 148.25, 136.35, 130.25, 129.16, 128.42, 116.45, 115.98, 110.66, 93.33, 64.11.

### 2-((4-methoxypyridin-2-yl)amino)-4-phenylnicotinonitrile (4f)

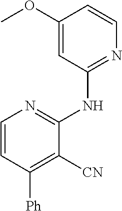 [SMILES: COc1ccnc(Nc2nccc(-c3ccccc3)c2C#N)c1]

General procedure C was followed using 2-chloro-4-phenylnicotinonitrile (100 mg, 1.0 equiv), 4-methoxypyridin-2-amine (70 mg, 1.2 equiv), palladium(II) acetate (5.2 mg, 5 mol %), BINAP (17.6 mg, 6 mol %), cesium carbonate (460 mg, 3.0 equiv), and 3 mL of dry toluene. The compound was purified by column chromatography on silica gel using pure hexanes. Removal of solvent afforded product as white crystals (122 mg, 87%). M.p.: 144-145° C. 1 H NMR (CDCl₃, 500 MHz, 21° C.): δ 8.45 (d, J=5.2 Hz, 1H), 8.14 (d, J=5.7 Hz, 1H), 8.11 (s, 1H), 8.02 (s, 1H), 7.64-7.58 (m, 2H), 7.55-7.49 (m, 3H), 6.95 (d, J=5.2 Hz, 1H), 6.57 (dd, J=5.7, 2.1 Hz, 1H), 3.91 (s, 3H). ¹³C{¹H} NMR (CDCl₃, 126 MHz, 21° C.): δ 167.45, 155.87, 155.14, 153.85, 151.20, 149.06, 136.43, 130.22, 129.16, 128.44, 115.99, 115.86, 105.72, 99.00, 55.49.

### 2-((4-bromopyridin-2-yl)amino)-4-phenylnicotinonitrile (4g)

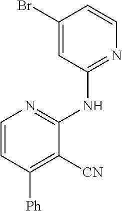 [SMILES: N#Cc1c(-c2ccccc2)ccnc1Nc1cc(Br)ccn1]

General procedure C was followed using 2-chloro-4-phenylnicotinonitrile (200 mg, 1.0 equiv), 4-bromopyridin-2-amine (267 mg, 1.2 equiv), tris(dibenzylideneacetone)dipalladium(0) (11 mg, 2.5 mol %), 1,1′-bis(bis(dimethylamino)phosphino)ferrocene (DMAPF, 12 mg, 6 mol %), sodium tert-butoxide (56 mg, 1.25 equiv), and 3 mL of dry toluene. The compound was purified by column chromatography on silica gel using 20% EtOAc in hexanes. Removal of solvent afforded product as white crystals (60 mg, 37%). M.p.: 166-167° C. ¹H NMR (CDCl₃, 500 MHz, 21° C.): δ 8.76 (s, 1H), 8.50 (d, J=5.2 Hz, 1H), 8.13 (d, J=5.3 Hz, 1H), 8.07 (s, 1H), 7.66-7.58 (m, 2H), 7.58-7.50 (m, 3H), 7.17 (dd, J=5.3, 1.6 Hz, 1H), 7.01 (d, J=5.2 Hz, 1H). ¹³C{¹H} NMR (CDCl₃, 126 MHz, 21° C.): δ 155.37, 155.25, 153.05, 151.25, 148.67, 136.24, 134.25, 130.35, 129.22, 128.44, 121.96, 116.48, 116.39, 115.83, 93.61.6

### Synthesis of 2-((3,5-dimethylphenyl)amino)nicotinonitrile Derivatives via Buchwald-Hartwig Coupling

### 2-((3,5-dimethylphenyl)amino)-6-methylnicotinonitrile (5a)

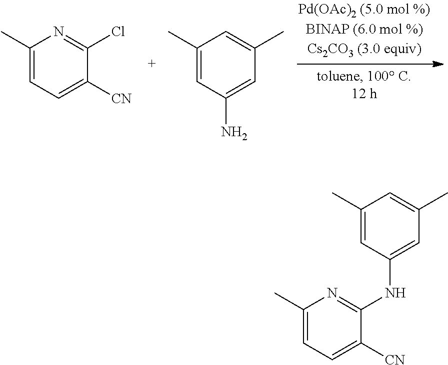

In a glove box, palladium(II) acetate (11.2 mg, 0.05 mmol, 5 mol %) and BINAP (37.7 mg, 0.06 mmol, 6.0 mol %) were dissolved in 2 mL of dry toluene in a 35 mL pressure tube. The mixture was stirred for 2 min before 2-chloro-6-methylnicotinonitrile (152 mg, 1.0 mmol, 1.0 equiv), dimethyl aniline (73 mL, 1.2 mmol, 1.2 equiv), and cesium carbonate (984 mg, 3.0 mmol, 3.0 equiv) were added to the pressure tube. Then, another 3 mL of dry toluene were added. The reaction was then removed from the glovebox and heated in an oil bath at 110° C. for 14 h. After the reaction was cooled to room temperature, the reaction mixture was filtered through Celite and rinsed with ethyl acetate. The filtrate was evaporated, and the crude product was purified by column chromatography on silica gel (hexane/ethyl acetate). Removal of solvent afforded product as white crystals (132 mg, 56%). ¹H NMR (CDCl₃, 500 MHz, 21° C.): δ 7.64 (d, J=7.8 Hz, 1H), 7.28 (s, 2H), 6.87 (s, 1H), 6.75 (s, 1H), 6.64 (d, J=7.8 Hz, 1H), 2.50 (s, 3H), 2.33 (s, 6H). ¹³C{1H} NMR (CDCl₃, 126 MHz, 21° C.): δ 162.91, 155.68, 141.59, 138.75, 138.70, 125.44, 118.20, 117.10, 113.72, 89.91, 25.29, 21.62. LRMS (EI): calc'd: 237; found: 236.

### 2-((3,5-dimethylphenyl)amino)-6-phenylnicotinonitrile (5b)

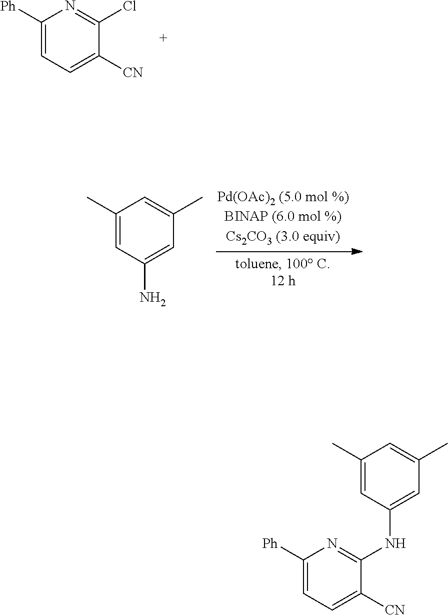

In a glove box, palladium(II) acetate (5.2 mg, 0.05 mmol, 5 mol %) and BINAP (17.6 mg, 6.0 mol %) were dissolved in 2 mL of dry toluene in a 35 mL pressure tube. The mixture was stirred for 2 min before 2-chloro-4-phenylnicotinonitrile (100 mg, 1.0 equiv), dimethylaniline (73 mL, 1.2 equiv), and cesium carbonate (460 mg, 3.0 equiv) were added to the pressure tube. Then, another 3 mL of dry toluene were added. The reaction was then removed from the glovebox and heated in an oil bath at 110° C. for 14 h. After the reaction was cooled to room temperature, the reaction mixture was filtered through Celite and rinsed with ethyl acetate. The filtrate was evaporated, and the crude product was purified by column chromatography on silica gel (hexane/ethyl acetate). Removal of solvent afforded product as white crystals (133 mg, 95%). M.p.: 135-136° C. ¹H NMR (CDCl₃, 500 MHz, 21° C.): δ 8.06 (dd, J=7.4, 2.0 Hz, 2H), 7.83 (d, J=8.1 Hz, 1H), 7.52-7.43 (m, 3H), 7.39 (s, 2H), 7.28 (d, J=8.1 Hz, 1H), 6.99 (s, 1H), 6.79 (s, 1H), 2.36 (s, 6H). ¹³C{1H} NMR (CDCl₃, 126 MHz, 21° C.): δ 159.49, 155.53, 142.20, 138.56, 137.84, 130.36, 128.82, 127.37, 125.33, 118.32, 116.91, 110.24, 91.05, 21.50. LRMS (EI): calc'd: 299; found: 298.

### Synthesis of 5-bromo-2-((3,5-dimethylphenyl)amino)nicotinonitrile (6-I)

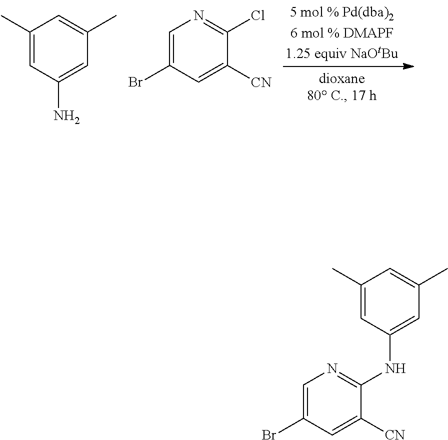

In a glove box, tris(dibenzylideneacetone)dipalladium(0) (23 mg, 0.05 mmol, 2.5 mol %) and DMAPF (25.3 mg, 0.06 mmol, 6 mol %) were dissolved in 2 mL of dry dioxane in a 35 mL pressure tube. The mixture was stirred for 2 min before 5-bromo-2-chloronicotinonitrile (217 mg, 1.0 mmol, 1.0 equiv), 3,5-dimethylaniline (125 mL, 1.0 mmol, 1.0 equiv), and sodium tert-butoxide (119 mg, 1.25 mmol, 1.25 equiv) were added to the pressure tube. Then, another 2 mL of dry dioxane was added. The reaction was then removed from the glovebox and heated in an oil bath at 70° C. for 15 h. After the reaction was cooled to room temperature, the reaction mixture was filtered through Celite and rinsed with ethyl acetate. The filtrate was evaporated, and the crude product was purified by column chromatography on silica gel (hexane/ethyl acetate) to afford pure product as an off-white solid (214 mg, 71%). M.p.: 145-147° C. ¹H NMR (CDCl₃, 500 MHz, 21° C.): δ 8.39 (s, 1H), 7.84 (s, 1H), 7.15 (s, 2H), 6.90 (s, 1H), 6.81 (s, 1H), 2.33 (s, 7H). ¹³C{1H} NMR (CDCl₃, 126 MHz, 21° C.): δ 154.89, 153.56, 143.17, 139.02, 137.80, 126.62, 119.20, 115.31, 107.29, 94.52, 21.57. LRMS (EI): calc'd: 301; found: 302.

### General Procedure D: Synthesis of 3-cyano-pyridine Derivatives via Suzuki Coupling

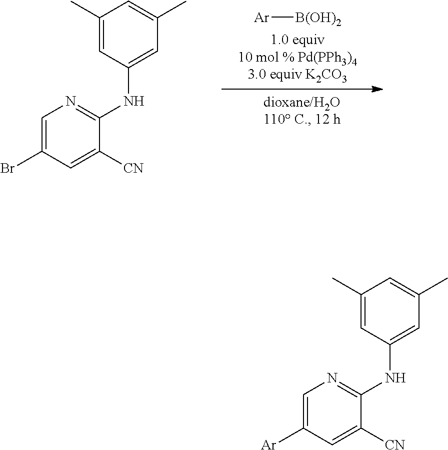

In a glove box, tetrakis(triphenylphosphine)palladium(0) (10 mol %), potassium carbonate (2.0 equiv), 5-bromo-2-((3,5-dimethylphenyl)amino)nicotinonitrile 6-I (1.0 equiv), aryl boronic acid (1.0 equiv), and 3 mL of dioxane were added in a 50 mL Schlenk tube. The reaction was then removed from the glovebox and charged with water under a constant flow of dry dinitrogen. Then, the Schlenk tube was sealed and heated in an oil bath at 70° C. for 15 h. After the reaction was cooled to room temperature, the reaction mixture was filtered through Celite and rinsed with ethyl acetate. The filtrate was evaporated, and the crude product was purified by column chromatography on silica gel (hexane/ethyl acetate) to afford pure product.

### 5-(3,5-dimethylphenyl)-2-((3,5-dimethylphenyl)amino)nicotinonitrile (6a)

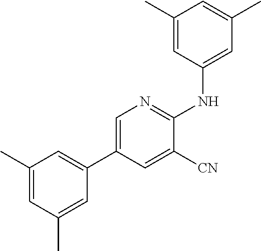

General procedure D was followed using 5-bromo-2-((3,5-dimethylphenyl)amino)nicotinonitrile 6-I (50 mg, 1.0 equiv), (3,5-dimethylphenyl)boronicacid (25 mg, 1.0 equiv), tetrakis(triphenylphosphine)palladium(0) (19 mg, 10 mol %), potassium carbonate (69 mg, 3.0 equiv), 2 mL of dioxane, and 0.5 mL of water. Removal of solvent afforded product as a white powder (40 mg, 74%). M.p.: 162-163° C. ¹H NMR (CDCl₃, 500 MHz, 21° C.): δ 8.61 (d, J=2.4 Hz, 1H), 7.96 (d, J=2.5 Hz, 1H), 7.23 (s, 2H), 7.11 (s, 2H), 7.03 (s, 1H), 6.94 (s, 1H), 6.79 (s, 1H), 2.38 (s, 6H), 2.34 (s, 6H). ¹³C{1H} NMR (CDCl₃, 126 MHz, 21° C.): δ 155.10, 150.61, 140.02, 139.03, 138.96, 138.30, 136.08, 129.75, 127.79, 126.23, 124.27, 119.01, 116.54, 93.27, 21.60, 21.53. LRMS (EI): calc'd: 327; found: 326.

### 5-(3,5-dimethoxyphenyl)-2-((3,5-dimethylphenyl)amino)nicotinonitrile (6b)

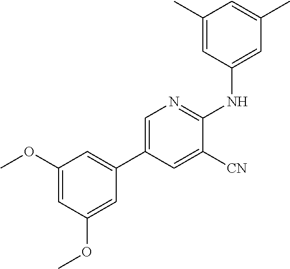

General procedure D was followed using 5-bromo-2-((3,5-dimethylphenyl)amino)nicotinonitrile 6-I (80 mg, 1.0 equiv), (3,5-dimethoxyphenyl)boronic acid (25 mg, 1.0 equiv), tetrakis(triphenylphosphine)palladium(0) (30 mg, 10 mol %), potassium carbonate (110 mg, 3.0 equiv), 2 mL of dioxane, and 0.5 mL of water. Removal of solvent afforded product as a white powder (80 mg, 84%). M.p.: 169-170° C. ¹H NMR (CDCl₃, 500 MHz, 21° C.): δ 8.61 (d, J=2.3 Hz, 1H), 7.95 (d, J=2.4 Hz, 1H), 7.23 (s, 2H), 6.96 (s, 1H), 6.80 (s, 1H), 6.61 (s, 2H), 6.48 (s, 1H), 3.85 (s, 6H), 2.35 (s, 6H). ¹³C{¹H} NMR (CDCl₃, 126 MHz, 21° C.): δ 161.57, 155.46, 151.01, 139.86, 138.96, 138.29, 127.46, 126.23, 118.98, 116.57, 104.67, 99.76, 93.11, 55.62, 21.61. LRMS (EI): calc'd: 359; found: 358.

### 5-(3,5-dichlorophenyl)-2-((3,5-dimethylphenyl)amino)nicotinonitrile (6c)

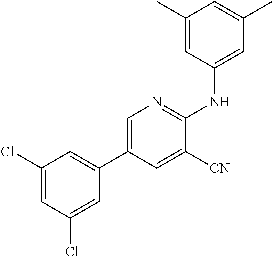 [SMILES: Cc1cc(C)cc(Nc2ncc(-c3cc(Cl)cc(Cl)c3)cc2C#N)c1]

General procedure D was followed using 5-bromo-2-((3,5-dimethylphenyl)amino)nicotinonitrile 6-I (80 mg, 1.0 equiv), (3,5-dichlorophenyl)boronic acid (51 mg, 1.0 equiv), tetrakis(triphenylphosphine)palladium(0) (30 mg, 10 mol %), potassium carbonate (110 mg, 3.0 equiv), 2 mL of dioxane, and 0.5 mL of water. Removal of solvent afforded product as a white powder (25 mg, 20%). M.p.: 191-192° C. ¹H NMR (CDCl₃, 500 MHz, 21° C.): δ 8.57 (d, J=2.4 Hz, 1H), 7.93 (d, J=2.5 Hz, 1H), 7.37 (s, 3H), 7.22 (s, 2H), 7.03 (s, 1H), 6.82 (s, 1H), 2.35 (s, 7H). ¹³C{¹H} NMR (CDCl₃, 126 MHz, 21° C.): δ 211.98, 155.85, 150.92, 139.73, 139.22, 139.04, 137.92, 136.02, 127.95, 126.60, 124.80, 119.21, 116.13, 93.40, 21.60. LRMS (EI): calc'd: 367; found: 366.

### 6-((3,5-dimethylphenyl)amino)-[3,4′-bipyridine]-5-carbonitrile (6d)

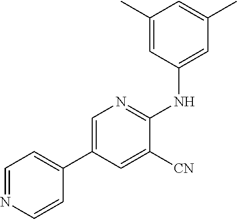 [SMILES: Cc1cc(C)cc(Nc2ncc(-c3ccncc3)cc2C#N)c1]

General procedure D was followed using 5-bromo-2-((3,5-dimethylphenyl)amino)nicotinonitrile 6-I (100 mg, 1.0 equiv), pyridin-4-ylboronic acid (41 mg, 1.0 equiv), tetrakis(triphenylphosphine)palladium(0) (38 mg, 10 mol %), potassium carbonate (137 mg, 3.0 equiv), 3 mL of dioxane, and 0.75 mL of water. Removal of solvent afforded product as a white powder (80 mg, 81%). M.p.: 185-186° C. ¹H NMR (CDCl₃, 500 MHz, 21° C.): δ 8.71-8.67 (m, 3H), 8.04 (d, J=2.5 Hz, 1H), 7.47-7.38 (m, 2H), 7.23 (s, 2H), 7.08 (s, 1H), 6.83 (s, 1H), 2.35 (s, 6H). ¹³C{1H} NMR (CDCl₃, 126 MHz, 21° C.): δ 156.27, 151.04, 150.84, 143.50, 139.74, 139.06, 137.81, 126.76, 124.19, 120.46, 119.39, 116.12, 93.52, 21.59. LRMS (EI): calc'd: 300; found: 299.

### 6-((3,5-dimethylphenyl)amino)-[3,3′-bipyridine]-5-carbonitrile (6e)

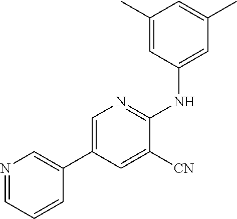 [SMILES: Cc1cc(C)cc(Nc2ncc(-c3cccnc3)cc2C#N)c1]

General procedure D was followed using 5-bromo-2-((3,5-dimethylphenyl)amino)nicotinonitrile 6-I (71 mg, 1.0 equiv), pyridin-3-ylboronic acid (29 mg, 1.0 equiv), tetrakis(triphenylphosphine)palladium(0) (27 mg, 10 mol %), potassium carbonate (97 mg, 3.0 equiv), 2 mL of dioxane, and 0.5 mL of water. Removal of solvent afforded product as a yellow powder (mg, 79%). M.p.: 93-95° C. ¹H NMR (CDCl₃, 500 MHz, 21° C.): δ 8.81 (s, 1H), 8.65 (d, J=4.6 Hz, 1H), 8.64 (s, 1H), 8.00 (s, 1H), 7.87 (d, J=7.9 Hz, 1H), 7.46 (t, J=6.7, 5.9 Hz, 1H), 7.24 (s, 2H), 7.05 (s, 1H), 6.84 (app t, 1H), 2.36 (s, 7H). ¹³C{¹H} NMR (CDCl₃, 126 MHz, 21° C.): δ 155.75, 150.83, 148.31, 146.64, 139.67, 138.92, 137.78, 134.18, 132.34, 126.51, 124.17, 123.62, 119.16, 116.04, 93.42, 21.46. LRMS (EI): calc'd: 300; found: 299.

### Replacements NH With O and CH₂ Linkers

### 2-(3,5-dimethylphenoxy)-5-phenylnicotinonitrile (7a)

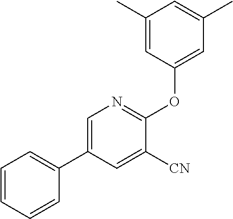 [SMILES: Cc1cc(C)cc(Oc2ncc(-c3ccccc3)cc2C#N)c1]

Under N₂, a 100 mL Schlenk flask was loaded with 2-bromo-5-phenylnicotinonitrile (500 mg, 1.94 mmol), 3,5-dimethylphenol (284 mg, 2.32 mmol, 1.2 equiv.), sodium hydride (93 mg, 3.88 mmol, 2.0 equiv.), 20 mL THF and a magnetic stir bar. The solution was heated to 60° C. and stirred for 3 hours. Then, the solvent was removed giving a light-yellow crude product. The crude product was purified by chromatography (EtOAc:hexane=1:10) and recrystallized from DCM/n-hexane. The product was formed as white crystals. (339 mg, 1.13 mmol, 58.2%). M.p.: 123-124° C. ¹H NMR (CDCl₃, 500 MHz, 21° C.): δ 8.53 (s, 1H), 8.17 (s, 1H), 7.46-7.52 (m, 4H), 7.41-7.46 (m, 1H), 6.93 (s, 1H), 6.84 (s, 2H), 2.36 (s, 6H). ¹³C{1H} NMR (CDCl₃, 126 MHz, 21° C.): 6163.44, 152.94, 150.21, 142.05, 140.01, 135.78, 132.05, 129.75, 128.95, 128.10, 127.13, 119.51, 115.29, 97.97, 21.74. LRMS (EI): calc'd: 300; found: 300.

### 2-(3,5-dimethylbenzyl)-5-phenylnicotinonitrile (7b)

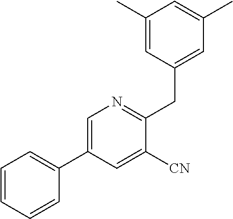 [SMILES: Cc1cc(C)cc(Cc2ncc(-c3ccccc3)cc2C#N)c1]

In a glovebox, a 20 ml scintillation vial was loaded with zinc dust (616 mg, 9.47 mmol, 5 equiv.), 3 mL of THF, and a micro magnetic stir bar. In another vial, 3,5-dimethylbenzyl bromide (490 mg, 2.46 mmol, 1.3 equiv.) was dissolved in 3 mL of THF. Then, 3,5-dimethylbenzyl bromide solution was added dropwise to the previous solution with a suspension of zinc dust. The mixture was stirred in room temperature for 10 minutes and filtered through celite, then added to a new vial loaded with 2-bromo-5-phenylnicotinonitrile (0.500 g, 1.94 mmol, 1 equiv.), Pd(PPh₃)₄ (17.6 mg, 0.08 equiv.), 5 mL of THF and a micro magnetic stir bar. The mixture was let stir overnight before filtered through a short alumina column. The product was purified by recrystallization with DCM/pentane. White needle-like crystals were formed. (510 mg, 1.71 mmol, 88.3%). M.p.: 127-128° C. ¹H NMR (CDCl₃, 500 MHz, 21° C.): δ 8.95 (s, 1H), 8.08 (s, 1H), 7.43-7.55 (m, 5H), 7.03 (s, 2H), 6.88 (s, 1H), 4.34 (s, 2H), 2.29 (s, 6H). ¹³C{1H} NMR (CDCl₃, 126 MHz, 21° C.): 6162.34, 151.30, 138.72, 138.45, 137.52, 135.63, 134.72, 129.55, 129.11, 128.82, 127.02, 117.24, 109.14, 42.75, 21.44. LRMS (EI): calc'd: 298; found: 298.

(Note: Zinc dust was activated by stirring in 1 mol/L HCl (aq.) for 10 minutes, then was washed with ether and dried in glovebox under vacuo.)

### Synthesis of 3-amino-6-phenylpyrazine-2-carbonitrile Derivatives via Chan-Lam Coupling

### 3-((3,5-dimethylphenyl)amino)-6-phenylpyrazine-2-carbonitrile (8a)

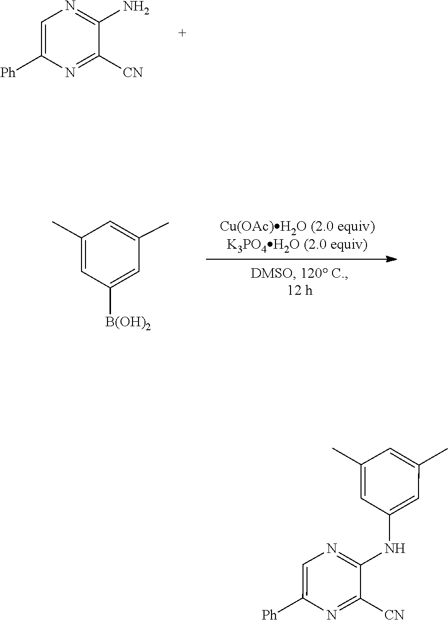

In a 50 mL round bottom flask, 3-amino-6-phenylpyrazine-2-carbonitrile (392 mg, 2.0 mmol, 1.0 equiv), (3,5-dimethylphenyl)boronic acid (300 mg, 2.0 mmol, 1.0 equiv), copper(II) acetate monohydrate (800 mg, 4.0 mmol, 2.0 equiv), potassium phosphate monohydrate (920 mg, 4.0 mmol, 2.0 equiv), and 10 mL of DMSO were loaded with a stir bar. The solution has a dark green color. The reaction was heated in an oil bath at 120° C. for 12 h. After the reaction was cooled to room temperature, the reaction mixture was diluted by adding 50 mL of EtOAc, then washed with brine. The organic layer was dried with sodium sulfate and evaporated. The crude product was purified by column chromatography on silica gel (hexane/ethyl acetate) to afford pure product as an off-white solid (53 mg, 9%). ¹H NMR (CDCl₃, 500 MHz, 21° C.): δ 8.78 (s, 1H), 7.92 (d, J=7.1 Hz, 2H), 7.49 (t, J=7.4 Hz, 2H), 7.44 (t, J=7.3 Hz, 1H), 7.21 (s, 2H), 7.03 (s, 1H), 6.84 (s, 1H), 2.36 (s, 6H). ¹³C{¹H} NMR (CDCl₃, 126 MHz, 21° C.): δ 152.40, 143.95, 143.03, 139.16, 137.42, 135.17, 129.48, 129.24, 126.80, 125.95, 119.06, 115.31, 114.41, 21.59. LRMS (EI): calc'd: 300; found: 299.

### 3-(bis(3,5-dimethylphenyl)amino)-6-phenylpyrazine-2-carbonitrile (8a-s)

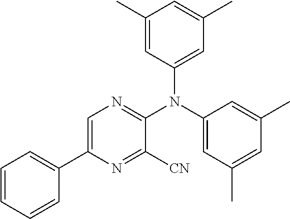 [SMILES: Cc1cc(C)cc(N(c2cc(C)cc(C)c2)c2ncc(-c3ccccc3)nc2C#N)c1]

The procedure to synthesize 3-((3,5-dimethylphenyl)amino)-6-phenylpyrazine-2-carbonitrile (f) also generated an N,N-diaryl byproduct, which was isolated as well. Column chromatography also afforded side product as a white solid (16 mg, 4%). ¹H NMR (CDCl₃, 500 MHz, 21° C.): δ 8.75 (s, 1H), 7.94 (d, J=7.0 Hz, 2H), 7.49 (t, J=7.2 Hz, 2H), 7.46-7.42 (m, 1H), 6.90 (s, 2H), 6.80 (s, 4H), 2.30 (s, 13H). ¹³C{1H} NMR (CDCl₃, 126 MHz, 21° C.): δ 152.39, 143.92, 143.01, 139.14, 137.43, 135.16, 129.47, 129.23, 126.78, 125.94, 119.04, 115.30, 114.40, 21.58. LRMS (EI): calc'd: 404; found: 404.

### 3-((3,5-dimethoxyphenyl)amino)-6-phenylpyrazine-2-carbonitrile (8b)

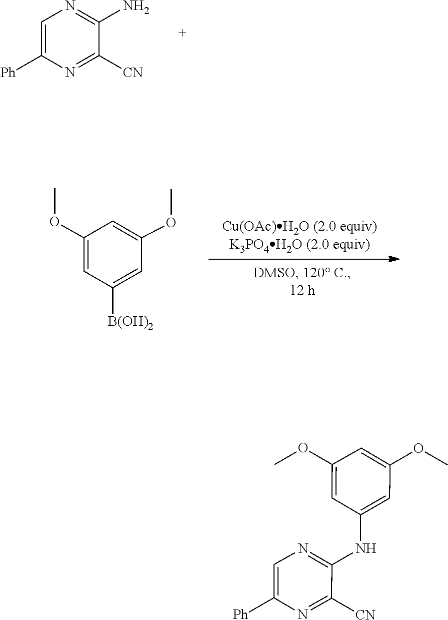 [SMILES: N#Cc1nc(-c2ccccc2)cnc1N ; COc1cc(OC)cc(B(O)O)c1 ; COc1cc(Nc2ncc(-c3ccccc3)nc2C#N)cc(OC)c1]

In a 50 mL round bottom flask, 3-amino-6-phenylpyrazine-2-carbonitrile (392 mg, 2.0 mmol, 1.0 equiv), (3,5-dimethoxyphenyl)boronic acid (364 mg, 2.0 mmol, 1.0 equiv), copper(II) acetate monohydrate (800 mg, 4.0 mmol, 2.0 equiv), potassium phosphate monohydrate (920 mg, 4.0 mmol, 2.0 equiv), and 10 mL of DMSO were added together with a stir bar. The reaction was heated in an oil bath at 120° C. for 12 h. After the reaction was cooled to room temperature, the reaction mixture was diluted by adding 50 mL of EtOAc, then washed with brine. The organic layer was dried with sodium sulfate and evaporated. The crude product was purified by column chromatography on silica gel (hexane/ethyl acetate) to afford pure product as an off-white solid (73 mg, 11%). ¹H NMR (CDCl₃, 500 MHz, 21° C.): δ 8.79 (s, 1H), 7.92 (d, J=7.5 Hz, 2H), 7.50 (t, J=7.5 Hz, 2H), 7.44 (t, J=7.2 Hz, 1H), 7.09 (s, 1H), 6.84 (d, J=2.1 Hz, 2H), 6.29 (s, 1H), 3.83 (s, 6H). ¹³C{1H} NMR (CDCl₃, 126 MHz, 21° C.): δ 161.36, 152.08, 144.24, 142.86, 139.40, 135.02, 129.61, 129.26, 126.01, 115.19, 114.69, 99.30, 96.61, 55.62. LRMS (EI): calc'd: 332; found: 332.

### Synthesis of Quinoline and 1,8-Naphthyridine Derivatives

### 2-((3,5-dimethylphenyl)amino)quinoline-3-carbonitrile (9a)

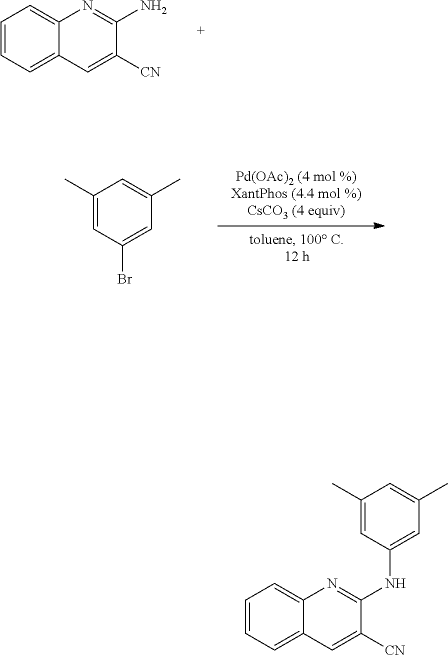

In a glove box, palladium(II) acetate (9.0 mg, 0.04 mmol, 4 mol %) and xantphos (25.4 mg, 0.044 mmol, 4.4 mol %) were dissolved in 2 mL of dry toluene in a 35 mL pressure tube. The mixture was stirred for 2 min before 2-aminoquinoline-3-carbonitrile (169 mg, 1.0 mmol, 1.0 equiv), 1-bromo-3,5-dimethylbenzene (185 mg, 1.0 mmol, 1.0 equiv), and cesium carbonate (1312 mg, 4.0 mmol, 4.0 equiv) were added to the pressure tube. Then, another 3 mL of dry toluene were added. The reaction was then removed from the glovebox and heated in an oil bath at 100° C. for 12 h. After the reaction was cooled to room temperature, the reaction mixture was filtered through Celite and rinsed with ethyl acetate. The filtrate was evaporated, and the crude product was purified by column chromatography on silica gel (hexane/ethyl acetate) to afford pure product as an off-white powder (253 mg, 93%). M.p.: 141-142° C. ¹H NMR (CDCl₃, 500 MHz, 21° C.): δ 8.34 (s, 1H), 7.82 (d, J=8.5 Hz, 1H), 7.70 (t, J=7.6 Hz, 1H), 7.66 (d, J=8.0 Hz, 1H), 7.47 (s, 2H), 7.36 (t, J=7.5 Hz, 1H), 7.05 (s, 1H), 6.79 (s, 1H), 2.37 (s, 7H). ¹³C{1H} NMR (CDCl₃, 126 MHz, 21° C.): δ 151.25, 144.31, 138.83, 133.26, 128.14, 127.67, 125.70, 124.59, 121.90, 118.18, 96.74, 21.69. LRMS (EI): calc'd: 273; found: 272.

### 2-((3-amino-5-methylphenyl)amino)quinoline-3-carbonitrile (9b)

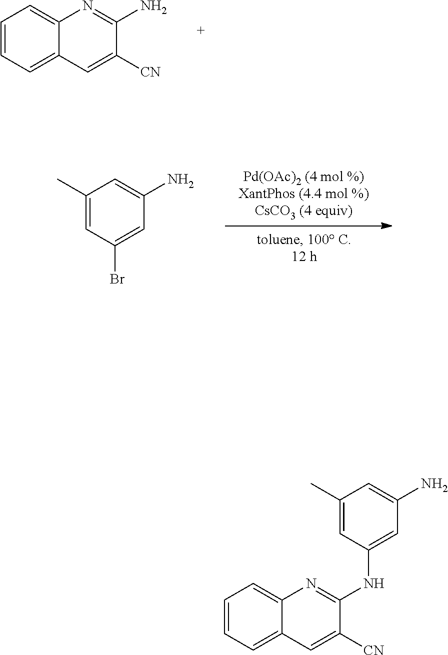

In a glove box, palladium(II) acetate (9.0 mg, 0.04 mmol, 4 mol %) and xantphos (25.4 mg, 0.044 mmol, 4.4 mol %) were dissolved in 2 mL of dry toluene in a 35 mL pressure tube. The mixture was stirred for 2 min before 2-aminoquinoline-3-carbonitrile (169 mg, 1.0 mmol, 1.0 equiv), 3-bromo-5-methylaniline (186 mg, 1.0 mmol, 1.0 equiv), and cesium carbonate (1312 mg, 4.0 mmol, 4.0 equiv) were added to the pressure tube. Then, another 3 mL of dry toluene were added. The reaction was then removed from the glovebox and heated in an oil bath at 100° C. for 12 h. After the reaction was cooled to room temperature, the reaction mixture was filtered through Celite and rinsed with ethyl acetate. The filtrate was evaporated, and the crude product was purified by column chromatography on silica gel (hexane/ethyl acetate) to afford pure product as an off-white powder (170 mg, 62%). M.p.: 159-160° C. ¹H NMR (CDCl₃, 500 MHz, 21° C.) δ 8.33 (s, 1H), 7.83 (d, J=8.6 Hz, 1H), 7.70 (t, J=7.8 Hz, 1H), 7.66 (d, J=7.8 Hz, 1H), 7.36 (t, J=7.5 Hz, 1H), 7.30 (s, 1H), 7.02 (s, 1H), 6.89 (s, 1H), 6.31 (s, 1H), 3.77 (s, 1H), 2.30 (s, 3H). ¹³C{1H} NMR (CDCl₃, 126 MHz, 21° C.): δ 147.19, 144.28, 139.99, 133.21, 128.15, 127.67, 124.59, 121.86, 116.40, 111.62, 111.35, 104.21, 21.76. LRMS (EI): calc'd: 274; found: 273.

### 2-((3,5-dimethylphenyl)amino)-1,8-naphthyridine-3-carbonitrile (9c)

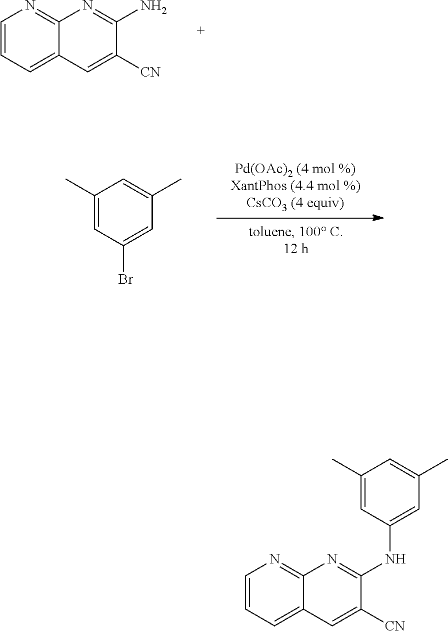

In a glove box, palladium(II) acetate (11.0 mg, 4 mol %) and xantphos (30 mg, 4.4 mol %) were dissolved in 2 mL of dry toluene in a 35 mL pressure tube. The mixture was stirred for 2 min before 2-amino-1,8-naphthyridine-3-carbonitrile (200 mg, 1.0 equiv), 1-bromo-3,5-dimethylbenzene (218 mg, 1.0 equiv), and cesium carbonate (1543 mg, 4.0 equiv) were added to the pressure tube. Then, another 3 mL of dry toluene were added. The reaction was then removed from the glovebox and heated in an oil bath at 100° C. for 12 h. After the reaction was cooled to room temperature, the reaction mixture was filtered through Celite and rinsed with ethyl acetate. The filtrate was evaporated, and the crude product was purified by column chromatography on silica gel (hexane/ethyl acetate) to afford pure product as an off-white powder (96 mg, 30%). M.p.: 182-183° C. ¹H NMR (CDCl₃, 500 MHz, 21° C.): δ 9.02 (dd, J=4.4, 2.0 Hz, 1H), 8.35 (s, 1H), 8.03 (d, J=9.9 Hz, 1H), 7.49 (s, 2H), 7.32 (dd, J=8.0, 4.4 Hz, 1H), 7.23 (s, 1H), 6.82 (s, 1H), 2.37 (s, 6H). δ ¹³C{¹H} NMR (CDCl₃, 126 MHz, 21° C.): δ 156.68, 156.35, 153.84, 145.01, 139.03, 137.94, 137.43, 126.63, 120.16, 118.84, 116.46, 115.63, 98.22, 21.69. LRMS (EI): calc'd: 274; found: 273.

### Synthesis of Other Pyridine Derivatives

### 6-chloro-[3,3′-bipyridine]-5-carbonitrile (10a)

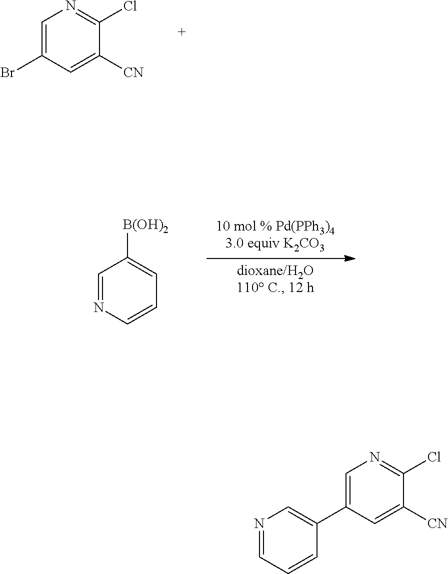 [SMILES: N#Cc1cc(Br)cnc1Cl ; OB(O)c1cccnc1 ; N#Cc1cc(-c2cccnc2)cnc1Cl]

In a glove box, tetrakis(triphenylphosphine)palladium(0) (1.6 g, 10 mol %), potassium carbonate (5.72 2.0 equiv), 5-bromo-2-((3,5-dimethylphenyl)amino)nicotinonitrile (1.0 equiv), aryl boronic acid (1.0 equiv), and 3 mL of dioxane were added in a 50 mL Schlenk tube. The reaction was then removed from the glovebox and heated in an oil bath at 11° C. for 12 h. After the reaction was cooled to room temperature, the reaction mixture was filtered through Celite and rinsed with ethyl acetate. The filtrate was evaporated, and the crude product was purified by column chromatography on silica gel (hexane/ethyl acetate) to afford pure product as a yellow solid (560 mg, 19%). ¹H NMR (CDCl₃, 500 MHz, 21° C.): δ 8.84 (d, J=2.2 Hz, 1H), 8.81 (d, J=2.5 Hz, 1H), 8.75 (dd, J=4.8, 1.5 Hz, 1H), 8.19 (d, J=2.5 Hz, 1H), 7.87 (dt, J=7.9, 2.0 Hz, 1H), 7.48 (dd, J=7.9, 4.8 Hz, 1H). ¹³C{1H} NMR (CDCl₃, 126 MHz, 21° C.): δ 152.34, 151.01, 150.87, 148.08, 140.73, 134.55, 133.05, 130.40, 124.27, 114.47, 111.35.

### 6-((3-bromo-5-methylphenyl)amino)-[3,3′-bipyridine]-5-carbonitrile (10b)

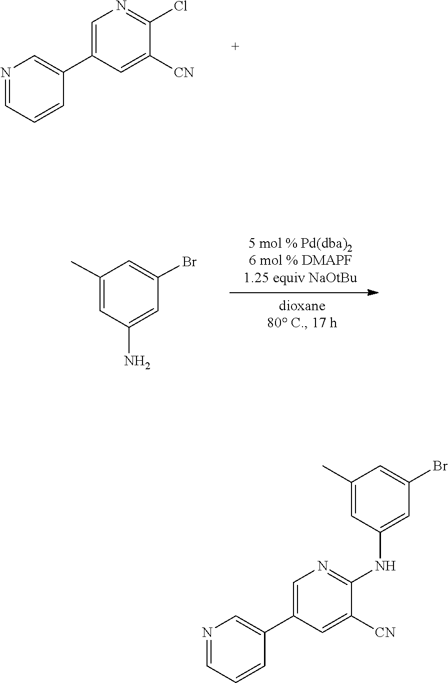

In a glove box, tris(dibenzylideneacetone)dipalladium(0) (6.4 mg, 2.5 mol %) and DMAPF (7 mg, 6 mol %) were dissolved in 2 mL of dry dioxane in a 15 mL pressure tube. The mixture was stirred for 2 min before 6-chloro-[3,3′-bipyridine]-5-carbonitrile (60 mg, 1.0 equiv), 3-bromo-5-methylaniline (52 mg, 1.0 equiv), and sodium tert-butoxide (33.5 mg, 1.25 equiv) were added to the pressure tube. Then, another 2 mL of dry dioxane was added. The reaction was then removed from the glovebox and heated in an oil bath at 70° C. for 15 h. After the reaction was cooled to room temperature, the reaction mixture was filtered through Celite and rinsed with ethyl acetate. The filtrate was evaporated, and the crude product was purified by column chromatography on silica gel (hexane/ethyl acetate) to afford pure product as an off-white powder (41 mg, 40%). M.p.: 168-169° C. ¹H NMR (CDCl₃, 500 MHz, 21° C.): δ 8.78 (d, J=2.2 Hz, 1H), 8.68-8.61 (m, 2H), 7.99 (s, 1H), 7.80 (d, J=10.5 Hz, 2H), 7.40 (dd, J=7.9, 4.9 Hz, 1H), 7.18 (s, 1H), 7.09 (s, 1H), 2.34 (s, 3H). ¹³C{¹H} NMR (CDCl₃, 126 MHz, 21° C.): δ 155.12, 150.68, 149.38, 147.51, 140.77, 139.86, 139.40, 133.67, 131.79, 127.89, 124.83, 124.01, 122.52, 120.88, 120.10, 115.93, 93.97, 21.40. LRMS (EI): calc'd: 364; found: 363.

### Synthesis of Cyclable 2-(pyridin-2-ylamino)nicotinonitrile Derivatives

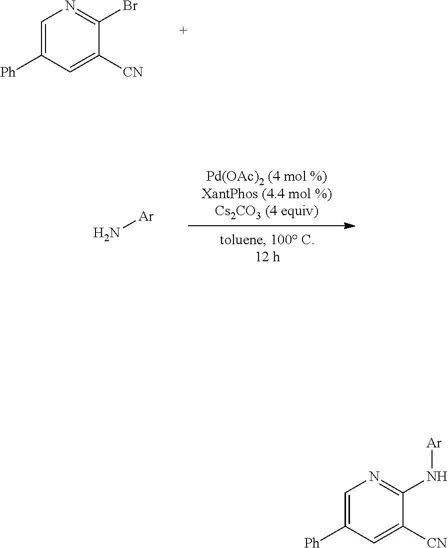

In a glove box, palladium(II) acetate (9.0 mg, 0.04 mmol, 4 mol %) and Xantphos (25.4 mg, 0.044 mmol, 4.4 mol %) were dissolved in 2 mL of dry toluene in a 35 mL pressure tube. The mixture was stirred for 2 min before 2-bromo-5-phenylnicotinonitrile (259 mg, 1.0 mmol, 1.0 equiv), aryl amine (1.2 mmol, 1.2 equiv), and cesium carbonate (1312 mg, 4.0 mmol, 4.0 equiv) were added to the pressure tube. Then, another 3 mL of dry toluene were added. The reaction was then removed from the glovebox and heated in an oil bath at 110° C. for 14 h. After the reaction was cooled to room temperature, the reaction mixture was filtered through Celite and rinsed with ethyl acetate. The filtrate was evaporated, and the crude product was purified by column chromatography on silica gel (hexane/ethyl acetate: 1:9).

### 2-((4-ethylpyridin-2-yl)amino)-5-phenylnicotinonitrile (11a)

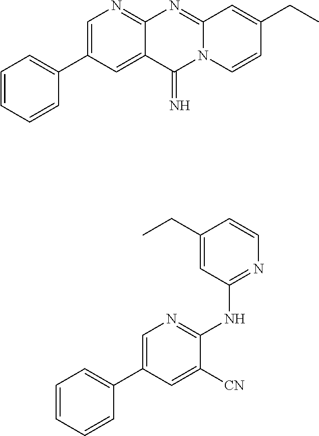 [SMILES: CCc1ccn2c(=N)c3cc(-c4ccccc4)cnc3nc2c1 ; CCc1ccnc(Nc2ncc(-c3ccccc3)cc2C#N)c1]

General procedure B was followed using 2-bromo-5-phenylnicotinonitrile (100 mg, 1.0 equiv), 4-ethylpyridin-2-amine (58 mg, 1.2 equiv), palladium(II) acetate (5 mg, 4 mol %), xantphos (10 mg, 4.4 mol %), cesium carbonate (500 mg, 4.0 equiv), and 3 mL of dry toluene. Removal of solvent afforded product as an orange powder (35 mg, 30%). M.p.: 122-123° C. ¹H NMR (CDCl₃, 500 MHz, 21° C.): δ 9.15 (s, 1H), 9.05 (d, J=7.2 Hz, 1H), 8.39 (s, 1H), 7.70 (s, 2H), 7.53 (s, 2H), 7.44 (s, 1H), 7.29 (s, 1H), 6.72 (d, J=8.3 Hz, 1H), 2.71 (q, J=7.5 Hz, 2H), 1.33 (t, J=7.5 Hz, 3H). ¹³C{1H} NMR (CDCl₃, 126 MHz, 21° C.): δ 165.00, 154.38, 153.40, 137.20, 131.16, 129.45, 129.41, 128.29, 127.48, 127.11, 126.53, 125.69, 122.70, 120.92, 114.49, 28.43, 13.17. LRMS (EI): calc'd: 300; found: 300.

### 2-((4-methoxypyridin-2-yl)amino)-5-phenylnicotinonitrile (11b)

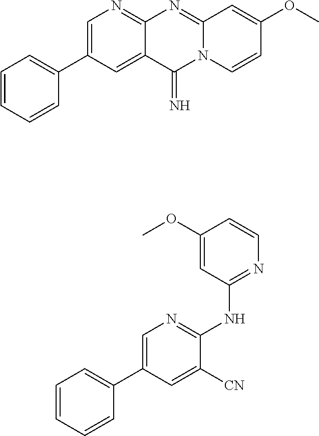 [SMILES: COc1ccn2c(=N)c3cc(-c4ccccc4)cnc3nc2c1 ; COc1ccnc(Nc2ncc(-c3ccccc3)cc2C#N)c1]

General procedure B was followed using 2-bromo-5-phenylnicotinonitrile (100 mg, 1.0 equiv), 4-methoxylpyridin-2-amine (58 mg, 1.2 equiv), palladium(II) acetate (5 mg, 4 mol %), xantphos (10 mg, 4.4 mol %), cesium carbonate (500 mg, 4.0 equiv), and 3 mL of dry toluene. Removal of solvent afforded product as an orange powder (72 mg, 62%). M.p.: 188-189° C. ¹H NMR (CDCl₃, 500 MHz, 21° C.): δ 9.08 (d, J=2.3 Hz, 1H), 9.01 (d, J=8.1 Hz, 1H), 8.28 (s, 1H), 7.66 (d, J=7.9 Hz, 2H), 7.58-7.47 (m, 2H), 7.42 (t, J=7.4 Hz, 1H), 6.68 (d, J=2.7 Hz, 1H), 6.52 (dd, J=8.1, 2.9 Hz, 1H), 3.94 (s, 3H). ¹³C{1H} NMR (CDCl₃, 126 MHz, 21° C.): δ 165.08, 156.46, 154.93, 154.29, 152.45, 137.31, 131.99, 131.18, 129.39, 128.19, 127.04, 126.56, 109.17, 108.48, 100.95. LRMS (EI): calc'd: 302; found: 302.

### 5-phenyl-2-(pyridin-2-ylamino)nicotinonitrile (11C)

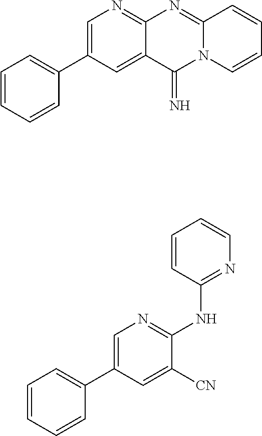 [SMILES: N=c1c2cc(-c3ccccc3)cnc2nc2ccccn12 ; N#Cc1cc(-c2ccccc2)cnc1Nc1ccccn1]

General procedure B was followed using 2-bromo-5-

phenylnicotinonitrile (200 mg, 1.0 equiv), pyridin-2-amine (87 mg, 1.2 equiv), palladium(II) acetate (7 mg, 4 mol %), xantphos (20 mg, 4.4 mol %), cesium carbonate (1013 mg, 4.0 equiv), and 5 mL of dry toluene. Removal of solvent afforded product as an orange powder (40 mg, 19%). ¹H NMR (CDCl₃, 500 MHz, 21° C.): δ 9.20-9.14 (m, 1H), 9.11 (d, J=7.1 Hz, 1H), 8.36 (s, 1H), 7.69 (d, J=7.5 Hz, 2H), 7.56-7.41 (m, 6H), 6.82 (t, J=7.3 Hz, 1H). ¹H NMR (DMSO-d₆, 500 MHz, 21° C.) δ 10.10 (s, 1H), 9.18-9.07 (m, 2H), 9.05 (d, J=7.0 Hz, 1H), 7.90 (d, J=7.1 Hz, 2H), 7.68 (ddd, J=9.0, 6.4, 1.6 Hz, 1H), 7.54 (t, J=7.8 Hz, 2H), 7.43 (t, J=7.4 Hz, 1H), 7.32 (d, J=8.6 Hz, 1H), 6.93 (ddd, J=7.8, 6.5, 1.5 Hz, 1H). ¹³C{1H} NMR (DMSO-d₆, 126 MHz, 21° C.): δ 154.21, 153.90, 152.72, 150.11, 136.81, 136.39, 132.33, 130.96, 129.14, 127.97, 127.94, 126.63, 125.49, 112.29, 110.46. LRMS (EI): calc'd: 272; found: 272.

### Ethyl 2-((3-cyano-5-phenylpyridin-2-yl)amino)isonicotinate (11d)

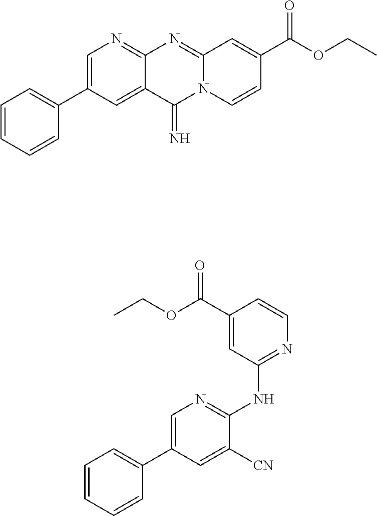 [SMILES: CCOC(=O)c1ccn2c(=N)c3cc(-c4ccccc4)cnc3nc2c1 ; CCOC(=O)c1ccnc(Nc2ncc(-c3ccccc3)cc2C#N)c1]

General procedure B was followed using 2-bromo-5-phenylnicotinonitrile (300 mg, 1.0 equiv), ethyl 2-aminoisonicotinate (231 mg, 1.2 equiv), palladium(II) acetate (11 mg, 4 mol %), xantphos (27 mg, 4.4 mol %), cesium carbonate (1.5 g, 4.0 equiv), and 5 mL of dry toluene. Removal of solvent afforded product as an orange powder (60 mg, 15%). ¹H NMR (CDCl₃, 500 MHz, 21° C.): δ 8.40 (d, J=5.2 Hz, 1H), 8.26-8.20 (m, 1H), 7.94 (ddd, J=8.0, 2.2, 1.0 Hz, 1H), 7.81 (dt, J=7.7, 1.3 Hz, 1H), 7.65-7.59 (m, 2H), 7.53 (dd, J=5.6, 2.1 Hz, 3H), 7.46 (t, J=7.9 Hz, 1H), 7.28 (s, 1H), 6.90 (d, J=5.2 Hz, 1H), 4.48-4.34 (m, 2H), 1.42 (t, J=7.1 Hz, 3H). ¹³C{1H} NMR (CDCl₃, 126 MHz, 21° C.): δ 166.41, 156.96, 155.06, 151.69, 138.99, 136.50, 131.53, 130.23, 129.21, 128.36, 125.32, 125.07, 121.99, 116.61, 115.40, 92.19, 61.30, 14.49. LRMS (EI): calc'd: 344; found: 344.

### 2-((4-(((tert-butyldimethylsilyl)oxy)methyl)pyridin-2-yl)amino)-5-phenylnicotinonitrile (11e)

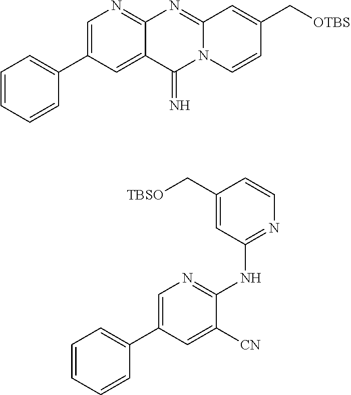 [SMILES: CC(C)(C)[Si](C)(C)OCc1ccn2c(=N)c3cc(-c4ccccc4)cnc3nc2c1 ; CC(C)(C)[Si](C)(C)OCc1ccnc(Nc2ncc(-c3ccccc3)cc2C#N)c1]

General procedure B was followed using 2-bromo-5-phenylnicotinonitrile (300 mg, 1.0 equiv), 4-(((tert-butyldimethylsilyl)oxy)methyl)pyridin-2-amine (331 mg, 1.2 equiv), palladium(II) acetate (11 mg, 4 mol %), xantphos (32 mg, 4.4 mol %), cesium carbonate (1520 mg, 4.0 equiv), and 5 mL of dry toluene. Removal of solvent afforded product as an yellow powder (398 mg, 82%). Column condition: 40% EtOAc in hexanes. ¹H NMR (CDCl₃, 500 MHz, 21° C.): δ 9.13 (s, 1H), 9.05 (d, J=7.9 Hz, 1H), 8.57 (s, 1H), 8.33 (s, 1H), 7.68 (d, J=7.8 Hz, 2H), 7.58-7.49 (m, 2H), 7.48-7.40 (m, 2H), 6.70 (d, J=7.6 Hz, 1H), 4.74 (s, 2H), 0.98 (s, 9H), 0.16 (s, 6H). ¹³C{1H} NMR (CDCl₃, 126 MHz, 21° C.): δ 154.60, 154.33, 150.78, 150.64, 137.17, 132.61, 131.13, 129.40, 128.30, 127.71, 127.11, 121.12, 110.95, 109.82, 63.43, 26.00, 18.49, −5.23.

### 5-phenyl-2-((4-(trifluoromethyl)pyridin-2-yl)amino)nicotinonitrile (11f)

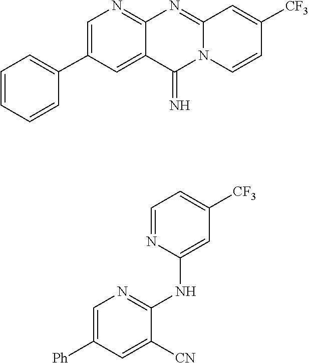 [SMILES: N=c1c2cc(-c3ccccc3)cnc2nc2cc(C(F)(F)F)ccn12 ; N#Cc1cc(-c2ccccc2)cnc1Nc1cc(C(F)(F)F)ccn1]

General procedure B was followed using 2-bromo-5-phenylnicotinonitrile (100 mg, 1.0 equiv), 4-(trifluoromethyl)pyridin-2-amine (75 mg, 1.2 equiv), palladium(II) acetate (4 mg, 4 mol %), xantphos (11 mg, 4.4 mol %), cesium carbonate (506 mg, 4.0 equiv), and 5 mL of dry toluene. Removal of solvent afforded product as an orange powder (101 mg, 77%). ¹H NMR (CDCl₃, 500 MHz, 21° C.): δ 8.81 (s, 1H), 8.79 (d, J=2.6 Hz, 1H), 8.50 (d, J=5.0 Hz, 1H), 8.12 (d, J=2.6 Hz, 1H), 8.07 (s, 1H), 7.57 (d, J=7.3 Hz, 2H), 7.53 (t, J=7.6 Hz, 2H), 7.49-7.43 (m, 1H), 7.24 (d, J=5.2 Hz, 1H). ¹³C{1H} NMR (CDCl₃, 126 MHz, 21° C.): δ 153.15, 152.98, 150.41, 149.30, 140.23, 140.08, 135.67, 129.69, 129.53, 128.61, 126.64, 115.60, 114.30, 114.27, 114.25, 114.22, 109.24, 109.21, 109.18, 109.15, 95.24. ¹⁹F NMR (471 MHz, CDCl3) δ −64.73.

### 5-phenyl-2-((4-((trimethylsilyl)ethynyl)pyridin-2-yl)amino)nicotinonitrile (11g)

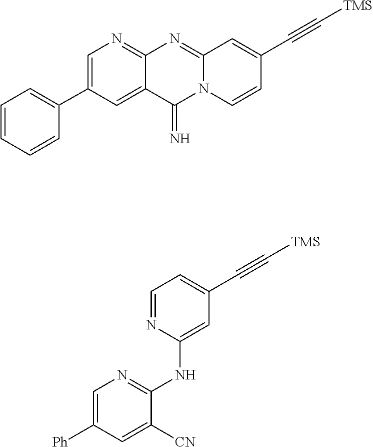 [SMILES: C[Si](C)(C)C#Cc1ccn2c(=N)c3cc(-c4ccccc4)cnc3nc2c1 ; C[Si](C)(C)C#Cc1ccnc(Nc2ncc(-c3ccccc3)cc2C#N)c1]

General procedure B was followed using 2-bromo-5-

phenylnicotinonitrile (327 mg, 1.0 equiv), 4-methoxylpyridin-2-amine (288 mg, 1.2 equiv), palladium(II) acetate (14 mg, 4 mol %), xantphos (42 mg, 4.4 mol %), cesium carbonate (1.9 g, 4.0 equiv), and 6 mL of dry toluene. Removal of solvent afforded product as an orange powder (90 mg, 16%). M.p.:116-120° C. ¹H NMR (CDCl₃, 500 MHz, 21° C.): δ 8.77 (d, J=2.4 Hz, 1H), 8.49 (s, 1H), 8.26 (d, J=5.1 Hz, 1H), 8.07 (d, J=2.6 Hz, 1H), 7.87 (s, 1H), 7.54 (d, J=7.0 Hz, 2H), 7.50 (t, J=7.7 Hz, 2H), 7.45-7.39 (m, 1H), 7.04 (dd, J=5.1, 1.4 Hz, 1H), 0.29 (s, 9H). ¹³C{1H} NMR (CDCl₃, 126 MHz, 21° C.): δ 153.41, 152.19, 150.46, 148.15, 140.01, 135.87, 133.20, 129.47, 129.08, 128.43, 127.17, 126.57, 121.23, 115.80, 115.33, 102.76, 99.47, 94.89, −0.09. LRMS (EI): calc'd: 368; found: 368.

### 2-((4-ethynylpyridin-2-yl)amino)-5-phenylnicotinonitrile (11h)

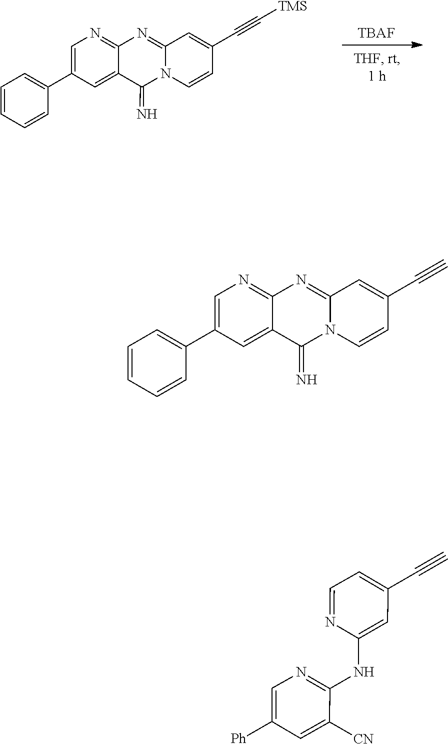 [SMILES: C[Si](C)(C)C#Cc1ccn2c(=N)c3cc(-c4ccccc4)cnc3nc2c1 ; "TBAF" ; C#Cc1ccn2c(=N)c3cc(-c4ccccc4)cnc3nc2c1 ; C#Cc1ccnc(Nc2ncc(-c3ccccc3)cc2C#N)c1]

In a 20 mL glass vial, 5-phenyl-2-((4-((trimethylsilyl)ethynyl)pyridin-2-yl)amino)nicotinonitrile (11g) (40 mg, 0.11 mmol) was dissolved in 5 mL of THF with a stir bar. To this solution, 1 mL of 1 M TBAF solution (1.0 mmol) in THF was added dropwise under room temperature. The colorless solution became bright orange. After 1 h of stirring, the solvent was removed and crude product was purified by column chromatography (silica hexanes:EtOAc 5:1), which afforded the desired compound as colorless crystals (20 mg, 63%). M.p.:104-108° C. ¹H NMR (CDCl₃, 500 MHz, 21° C.): δ 9.15 (d, J=2.4 Hz, 1H), 9.02 (d, J=7.6 Hz, 1H), 8.32 (d, J=2.4 Hz, 1H), 7.68 (d, J=7.1 Hz, 2H), 7.56 (d, J=1.8 Hz, 1H), 7.53 (t, J=7.6 Hz, 2H), 7.45 (t, J=7.4 Hz, 1H), 6.74 (dd, J=7.6, 1.8 Hz, 1H), 3.49 (s, 1H). ¹³C{1H} NMR (CDCl₃, 126 MHz, 21° C.): δ 154.45, 136.90, 133.45, 131.17, 130.26, 129.49, 128.55, 127.94, 127.18, 114.13, 84.97, 80.47.

### 5-phenyl-2-(pyrazin-2-ylamino)nicotinonitrile (11i)

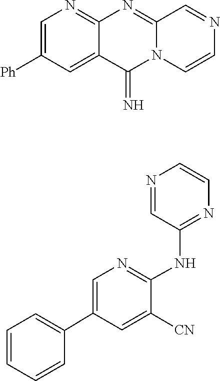 [SMILES: N=c1c2cc(-c3ccccc3)cnc2nc2cnccn12 ; N#Cc1cc(-c2ccccc2)cnc1Nc1cnccn1]

General procedure B was followed using 2-bromo-5-phenylnicotinonitrile (200 mg, 1.0 equiv), pyrazin-2-amine (88 mg, 1.2 equiv), palladium(II) acetate (7 mg, 4 mol %), xantphos (20 mg, 4.4 mol %), cesium carbonate (1013 mg, 4.0 equiv), and 4 mL of dry toluene. Removal of solvent afforded product as an orange powder (85 mg, 40%). M.p.: 166-167° C. ¹H NMR (CDCl₃, 500 MHz, 21° C.): δ 9.22 (s, 1H), 8.92 (s, 1H), 8.72 (s, 2H), 8.37 (s, 1H), 7.79 (d, J=5.0 Hz, 1H), 7.69 (d, J=7.6 Hz, 2H), 7.55 (t, J=7.7 Hz, 2H), 7.48 (t, J=7.7 Hz, 1H). ¹³C{1H} NMR (CDCl₃, 126 MHz, 21° C.): δ 154.67, 153.81, 148.93, 143.55, 141.91, 136.50, 134.92, 131.08, 129.58, 128.92, 128.45, 127.32, 126.66, 117.52. 1H NMR (500 MHz, dmso) δ 10.24 (s, 1H), 9.23 (s, 1H), 9.14 (d, J=2.6 Hz, 1H), 8.78 (s, 1H), 8.68 (d, J=5.1 Hz, 1H), 7.93 (d, J=7.6 Hz, 2H), 7.79 (d, J=5.0 Hz, 1H), 7.56 (t, J=7.7 Hz, 2H), 7.46 (t, J=7.3 Hz, 1H). LRMS (EI): calc'd: 273; found: 273.

### 5-(3-chlorophenyl)-2-((4-methylpyridin-2-yl)amino)nicotinonitrile (11j)

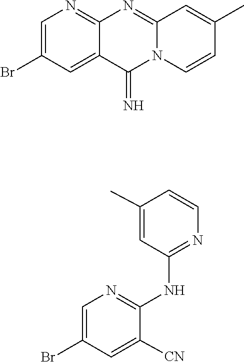 [SMILES: Cc1ccn2c(=N)c3cc(Br)cnc3nc2c1 ; Cc1ccnc(Nc2ncc(Br)cc2C#N)c1]

In a glove box, tris(dibenzylideneacetone)dipalladium(0) (23 mg, 2.5 mol %) and DMAPF (25 mg, 6 mol %) were dissolved in 2 mL of dry dioxane in a 35 mL pressure tube. The mixture was stirred for 2 min before 5-bromo-2-chloro-nicotinonitrile (217 mg, 1.0 equiv), 4-methylpyridin-2-amine (108 mg, 1.0 equiv), and sodium tert-butoxide (116 mg, 1.25 equiv) were added to the pressure tube. Then, another 3 mL of dry dioxane was added. The reaction was then removed from the glovebox and heated in an oil bath at 80° C. for 15 h. After the reaction was cooled to room temperature, the reaction mixture was filtered through Celite and rinsed with ethyl acetate. The filtrate was evaporated, and the crude product was purified by column chromatography on silica gel (85% EtOAc in hexanes) to afford pure product as a red solid (160 mg, 55%). ¹H NMR (CDCl₃, 500 MHz, 21° C.): δ 8.96 (d, J=6.4 Hz, 1H), 8.85 (s, 1H), 8.48 (d, J=13.7 Hz, 1H), 8.27 (s, 1H), 7.20 (s, 1H), 6.66 (d, J=7.6 Hz, 1H), 2.40 (s, 3H). LRMS (EI): calc'd:288; found: 289. The NMR shows a compound with modest purity, that was used without further purification in the next step.

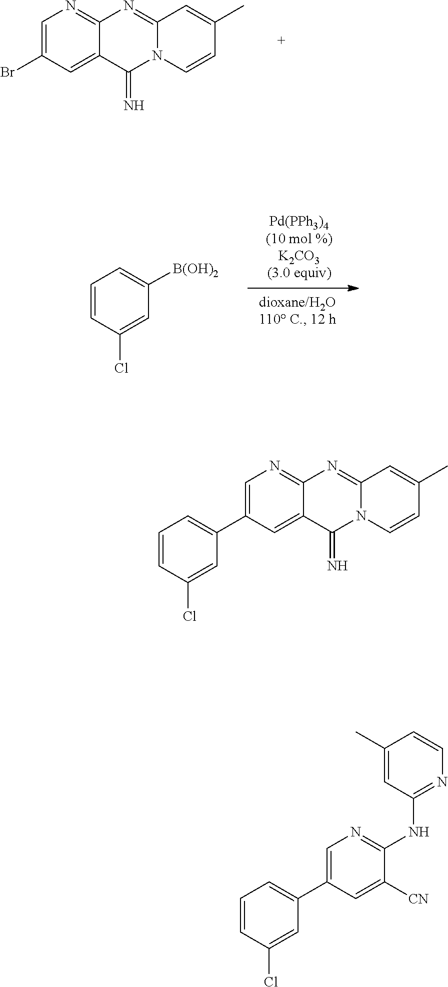 [SMILES: Cc1ccn2c(=N)c3cc(Br)cnc3nc2c1 ; OB(O)c1cccc(Cl)c1 ; Cc1ccn2c(=N)c3cc(-c4cccc(Cl)c4)cnc3nc2c1 ; Cc1ccnc(Nc2ncc(-c3cccc(Cl)c3)cc2C#N)c1]

In a glove box, tetrakis(triphenylphosphine)palladium(0) (19 mg, 10 mol %), potassium carbonate (46 mg, 2.0 equiv), 3-bromo-9-methyl-5H-dipyrido[1,2-a:2′,3′-d]pyrimidin-5-imine (50 mg, 1.0 equiv), 3-chlorophenyl boronic acid (33 mg, 1.2 equiv), and 3 mL of dioxane were added in a 50 mL Schlenk tube. The reaction was then removed from the glovebox and charged with water (0.5 mL) under a constant flow of dry dinitrogen. Then, the Schlenk tube was sealed and heated in an oil bath at 110° C. for 12 h. After the reaction was cooled to room temperature, the reaction mixture was filtered through Celite and rinsed with ethyl acetate. The filtrate was evaporated, and the crude product was purified by column chromatography on silica gel (hexane/ethyl acetate) to afford pure product. Removal of solvent afforded product as a white powder (21 mg, 38%). ¹H NMR (CDCl₃, 500 MHz, 21° C.): δ 9.08 (d, J=2.4 Hz, 1H), 9.02 (d, J=6.4 Hz, 1H), 8.57 (s, 1H), 8.28 (s, 1H), 7.65 (s, 1H), 7.55 (d, J=7.6 Hz, 1H), 7.45 (t, J=7.7 Hz, 1H), 7.40 (d, J=8.9 Hz, 1H), 7.24 (s, 1H), 6.67 (dd, J=7.5, 2.0 Hz, 1H), 2.41 (s, 3H). ¹³C{1H} NMR (CDCl₃, 126 MHz, 21° C.): δ 178.31, 153.94, 135.26, 133.29, 132.37, 130.53, 129.52, 128.16, 127.05, 125.10, 115.41, 85.24, 77.27, 77.02, 76.77, 21.45.

### 3-bromo-2,9-dimethyl-5H-dipyrido[1,2-a:2′,3′-d]pyrimidin-5-imine (11k)

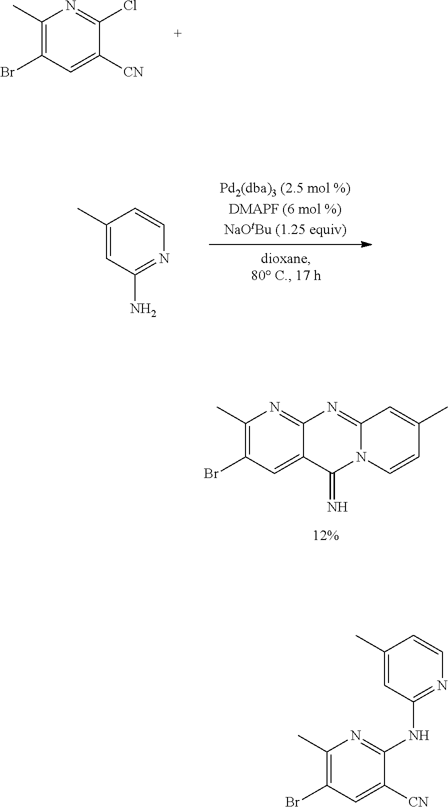

In a glove box, tris(dibenzylideneacetone)dipalladium(0) (38 mg, 2.5 mol %) and DMAPF (42 mg, 6 mol %) were dissolved in 2 mL of dry dioxane in a 35 mL pressure tube. The mixture was stirred for 2 min before 5-bromo-2-chloro-6-methylnicotinonitrile (386 mg, 1.0 equiv), 4-methylpyridin-2-amine (216 mg, 1.2 equiv), and sodium tert-butoxide (200 mg, 1.25 equiv) were added to the pressure tube. Then, another 4 mL of dry dioxane was added. The reaction was then removed from the glovebox and heated in an oil bath at 70° C. for 15 h. After the reaction was cooled to room temperature, the reaction mixture was filtered through Celite and rinsed with ethyl acetate. The filtrate was evaporated, and the crude product was purified by column chromatography on silica gel (85% EtOAc in hexanes) to afford pure product as a red solid (70 mg, 12%). ¹H NMR (CDCl₃, 500 MHz, 21° C.): δ 8.96 (d, J=7.7 Hz, 1H), 8.29 (s, 1H), 8.23 (s, 1H), 7.17 (s, 1H), 6.64 (dd, J=7.5, 2.0 Hz, 1H), 2.77 (s, 3H), 2.38 (s, 3H). The NMR shows a compound with modest purity, that was used without further purification in the next step.

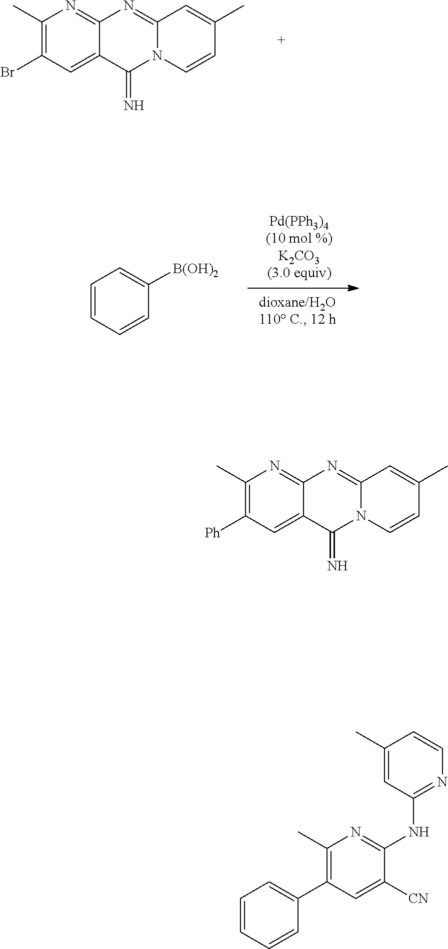

In a glove box, tetrakis(triphenylphosphine)palladium(0) (19 mg, 10 mol %), potassium carbonate (46 mg, 2.0 equiv), 3-bromo-2,9-dimethyl-5H-dipyrido[1,2-a:2′,3′-d]pyrimidin-5-imine (50 mg, 1.0 equiv), phenyl boronic acid (24 mg, 1.2 equiv), and 3 mL of dioxane were added in a 50 mL Schlenk tube. The reaction was then removed from the glovebox and charged with water (0.5 mL) under a constant flow of dry dinitrogen. Then, the Schlenk tube was sealed and heated in an oil bath at 110° C. for 12 h. After the reaction was cooled to room temperature, the reaction mixture was filtered through Celite and rinsed with ethyl acetate. The filtrate was evaporated, and the crude product was purified by column chromatography on silica gel (hexane/ethyl acetate) to afford pure product. Removal of solvent afforded product as a white powder (31 mg, 63%). M.p.: 136-137° C. ¹H NMR (CDCl₃, 500 MHz, 21° C.): δ 8.96 (d, J=7.5 Hz, 1H), 8.26 (s, 1H), 7.95 (s, 1H), 7.52 — 7.33 (m, 5H), 7.19 (s, 1H), 6.61 (d, J=5.5 Hz, 1H), 2.61 (s, 3H), 2.39 (s, 3H). ¹³C{1H} NMR (CDCl₃, 126 MHz, 21° C.): δ 162.92, 156.36, 153.90, 150.49, 147.36, 139.19, 133.89, 133.59, 129.24, 128.58, 127.76, 127.17, 124.02, 114.99, 107.72, 24.43, 21.41. LRMS (EI): calc'd: 300; found: 299.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

Those skilled in the art will recognize that numerous modifications can be made to the specific implementations described above. The implementations should not be limited to the particular limitations described. Other implementations may be possible.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or description that the steps are to be limited to a specific order, it is in no way intended that any particular order be inferred.

All publications and patents mentioned in the above specification are herein incorporated by reference in their entirety for all purposes. In the event of inconsistent usages between this document and those documents so incorporated by reference, the usage in the incorporated reference should be considered supplementary to that of this document; for irreconcilable inconsistencies, the usage in this document controls. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. A compound of the formula (I): 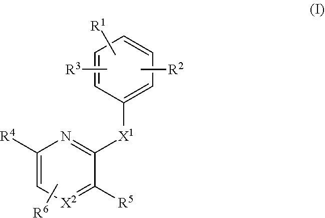
or a pharmaceutically acceptable salt, polymorph, prodrug, solvate or clathrate thereof, wherein:
R¹—R⁶ are each independently H, alkyl, aryl, arylalkyl, OR⁷, cyano, amino, amido or halo, wherein R⁷ is H, alkyl, arylalkyl or a protecting group or R⁴ and R⁶, together with the atoms to which they are attached, form an aryl or a heteroaryl group;
X¹ is X³, —O—X⁴—, or —N(R⁸)—X⁴—, wherein X³ is alkyl, X⁴ is a bond or alkyl, and R⁸ is H, alkyl, arylalkyl or acyl; and
X² is CR¹ or N;
wherein the compound is not:

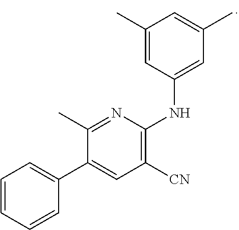 [SMILES: Cc1cc(C)cc(Nc2nc(C)c(-c3ccccc3)cc2C#N)c1]

2. The compound of claim 1, wherein R⁶ is alkyl or aryl.

3. (canceled)

4. (canceled)

5. (canceled)

6. The compound of claim 1, wherein R¹—R³ are each independently H, alkyl, OR⁷ or halo.

7. The compound of claim 1, wherein R⁵ is cyano or amido.

8. The compound of claim 1, wherein R⁴ is H or alkyl.

9. (canceled)

10. The compound of claim 1, wherein X¹ is —N(R⁸)—X⁴—.

11. (canceled)

12. (canceled)

13. The compound of claim 1, wherein the compound of the formula (I) is a compound of the formula (II): 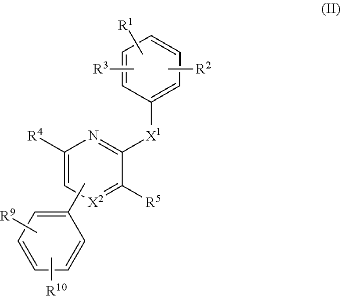
or a pharmaceutically acceptable salt, polymorph, prodrug, solvate or clathrate thereof, wherein:
R¹—R⁵, R⁹, and R¹⁰ are each independently H, alkyl, aryl, arylalkyl, OR⁷, cyano, amino, amido or halo, wherein R⁷ is H, alkyl, arylalkyl or a protecting group;
X¹ is X³, —O—X⁴—, or —N(R⁸)—X⁴—, wherein X³ is alkyl, X⁴ is a bond or alkyl, and R⁸ is H, alkyl, arylalkyl or acyl; and
X² is CR¹ or N.

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. The compound of claim 1, wherein the compound of the formula (I) is a compound of the formula (IIb) or (IIc):

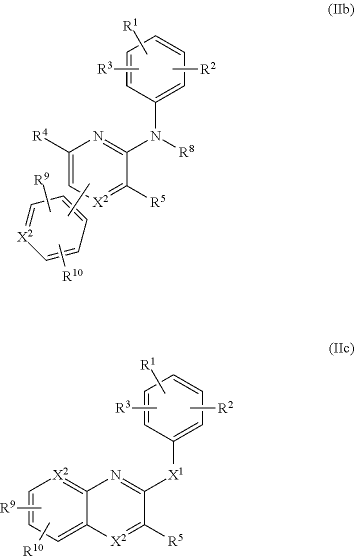

26. (canceled)

27. The compound claim 1, wherein the compound is selected from the group consisting of: 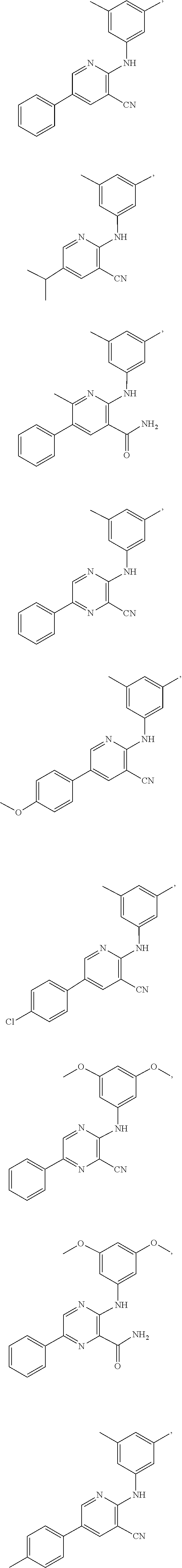 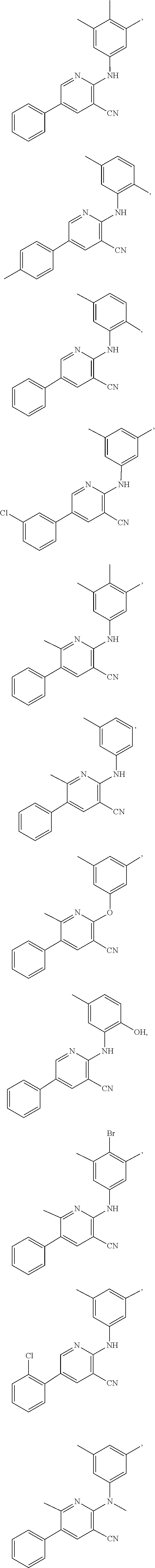 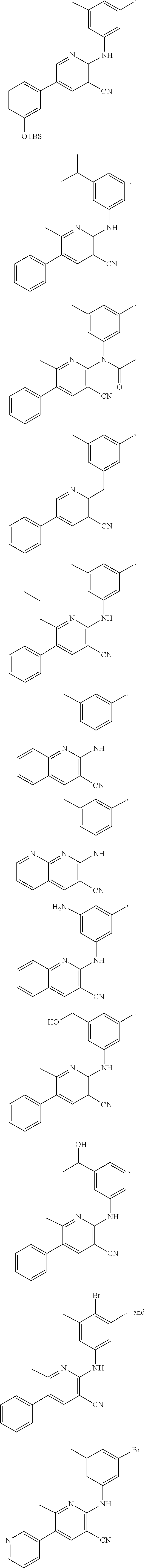
or a pharmaceutically acceptable salt, polymorph, prodrug, solvate or clathrate thereof.

28. A compound of the formula (III): 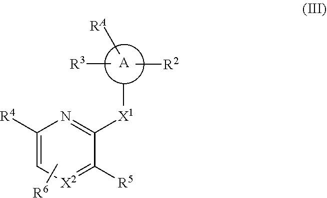
or a pharmaceutically acceptable salt, polymorph, prodrug, solvate or clathrate thereof, wherein:
A is aryl or heteroaryl;
R^{A} is H, alkyl, alkynyl, aryl, arylalkyl, OR⁷, C(O)OR⁷, cyano, amino, amido, or halo, wherein R⁷ is H, alkyl, arylalkyl or a protecting group;
R²—R⁶ are each independently H, alkyl, aryl, arylalkyl, OR⁷, cyano, amino, amido, or halo, wherein R⁷ is H, alkyl, arylalkyl or a protecting group;
X¹ is X³, —O—X⁴—, or —N(R⁸)—X⁴—, wherein X³ is alkyl (e.g., CH₂), X⁴ is a bond or alkyl, and R⁸ is H, alkyl, arylalkyl or acyl; and
X² is CR¹ or N.

29. (canceled)

30. (canceled)

31. (canceled)

32. (canceled)

33. The compound of claim 28, wherein A is 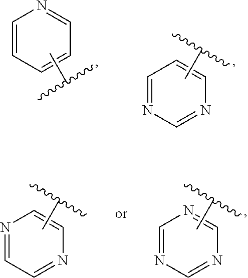
wherein each group can be substituted with R^{A}, R², and R³.

34. The compound of claim 28, wherein the compound is selected from the group consisting of: 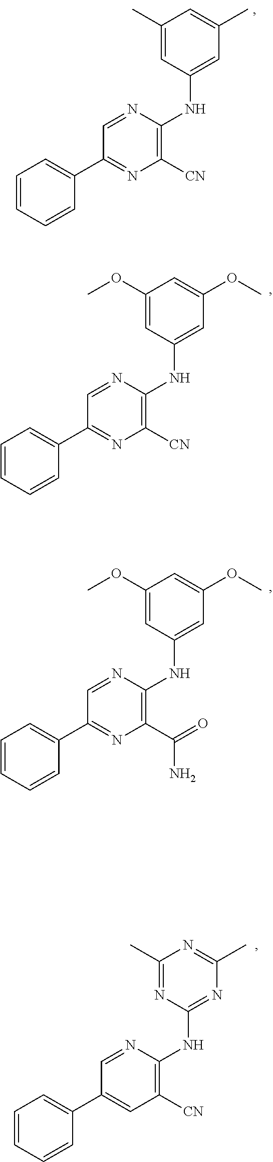 [SMILES: Cc1cc(C)cc(Nc2ncc(-c3ccccc3)nc2C#N)c1 ; COc1cc(Nc2ncc(-c3ccccc3)nc2C#N)cc(OC)c1 ; COc1cc(Nc2ncc(-c3ccccc3)nc2C(N)=O)cc(OC)c1 ; Cc1nc(C)nc(Nc2ncc(-c3ccccc3)cc2C#N)n1] 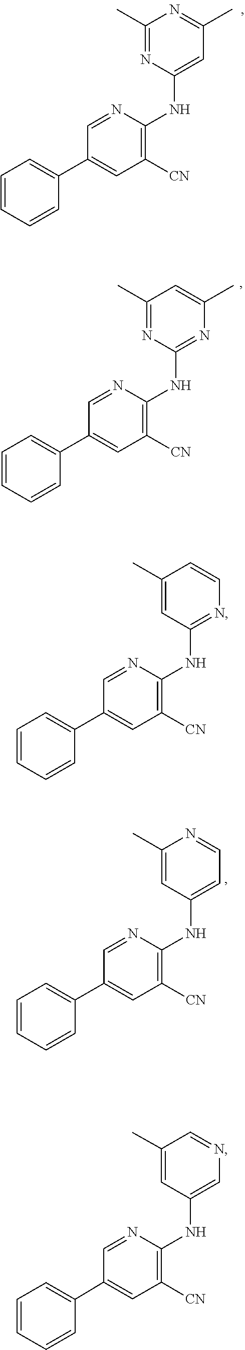 [SMILES: Cc1cc(Nc2ncc(-c3ccccc3)cc2C#N)nc(C)n1 ; Cc1cc(C)nc(Nc2ncc(-c3ccccc3)cc2C#N)n1 ; Cc1ccnc(Nc2ncc(-c3ccccc3)cc2C#N)c1 ; Cc1cc(Nc2ncc(-c3ccccc3)cc2C#N)ccn1 ; Cc1cncc(Nc2ncc(-c3ccccc3)cc2C#N)c1] 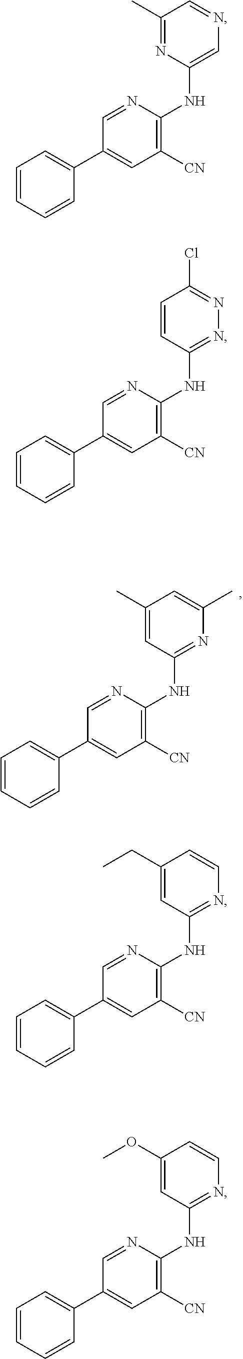 [SMILES: Cc1cncc(Nc2ncc(-c3ccccc3)cc2C#N)n1 ; N#Cc1cc(-c2ccccc2)cnc1Nc1ccc(Cl)nn1 ; Cc1cc(C)nc(Nc2ncc(-c3ccccc3)cc2C#N)c1 ; CCc1ccnc(Nc2ncc(-c3ccccc3)cc2C#N)c1 ; COc1ccnc(Nc2ncc(-c3ccccc3)cc2C#N)c1] 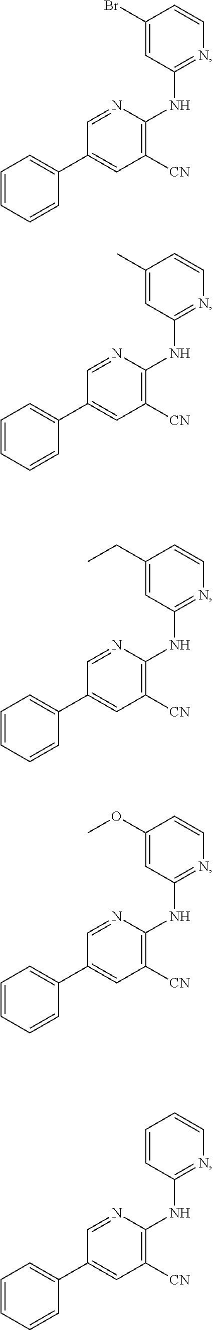 [SMILES: N#Cc1cc(-c2ccccc2)cnc1Nc1cc(Br)ccn1 ; Cc1ccnc(Nc2ncc(-c3ccccc3)cc2C#N)c1 ; CCc1ccnc(Nc2ncc(-c3ccccc3)cc2C#N)c1 ; COc1ccnc(Nc2ncc(-c3ccccc3)cc2C#N)c1 ; N#Cc1cc(-c2ccccc2)cnc1Nc1ccccn1] 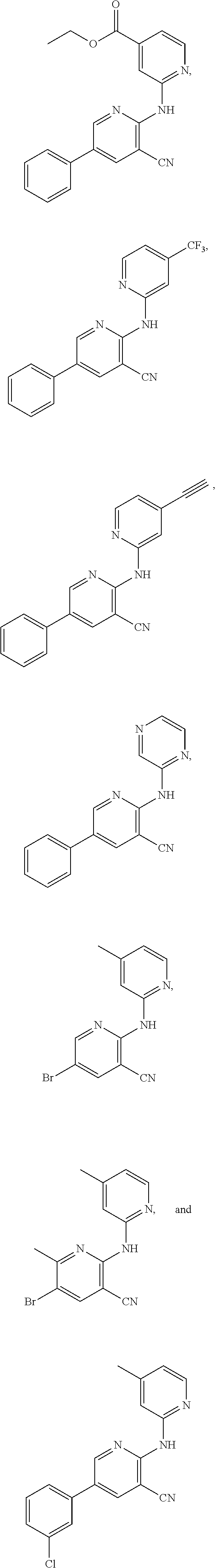
or a pharmaceutically acceptable salt, polymorph, prodrug, solvate or clathrate thereof

35. A pharmaceutical composition comprising a compound of claim 1 and one or more pharmaceutically acceptable carriers, diluents, excipients or combinations thereof.

36. A method for treating a tumor with constitutive activation of the Nrf2 pathway, the method comprising administering one or more compounds of claim 1.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. The method of claim 36, wherein the compound of the formula (I) is a compound of the formula (II): 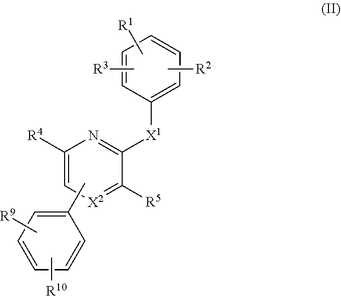
or a pharmaceutically acceptable salt, polymorph, prodrug, solvate or clathrate thereof, wherein:
R¹—R⁵, R⁹, and R¹⁰ are each independently H, alkyl, aryl, arylalkyl, OR⁷, cyano, amino, amido or halo, wherein R⁷ is H, alkyl, arylalkyl or a protecting group;
X¹ is X³, —O—X⁴—, or —N(R⁸)—X⁴—, wherein X³ is alkyl, X⁴ is a bond or alkyl, and R⁸ is H, alkyl, arylalkyl or acyl; and
X² is CR¹ or N.

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. The method of claim 36, wherein the compound is selected from the group consisting of: 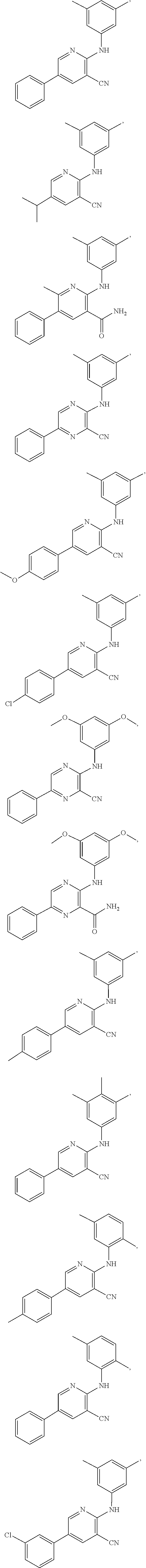 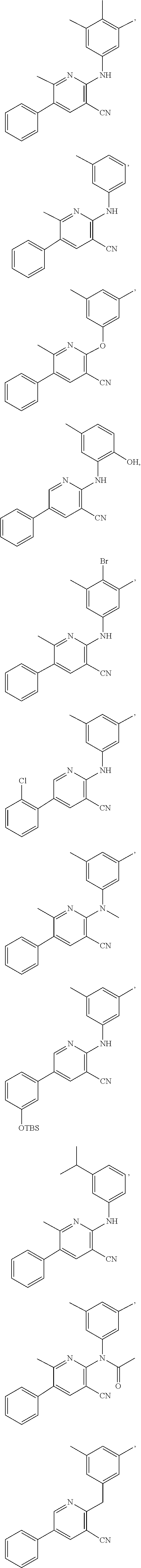 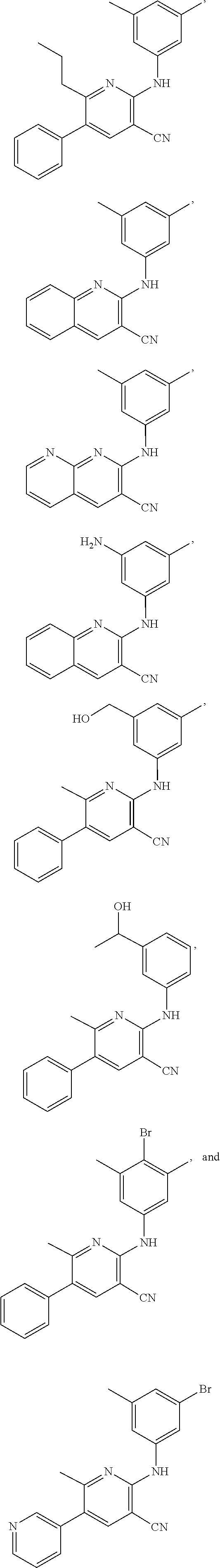
or a pharmaceutically acceptable salt, polymorph, prodrug, solvate or clathrate thereof.

61. The method of claim 36, wherein the tumor results from mutations in Keap1, NFE2L2 or epigenetic changes.

62. (canceled)

63. A method for treating non-small cell lung cancer (NSCLC) comprising administering one or more compounds of claim 1 or the pharmaceutical composition comprising said compound to a subject in need thereof.

64. A method for treating a tumor with constitutive activation of the Nrf2 pathway, the method comprising administering one or more compounds of the formula (III): 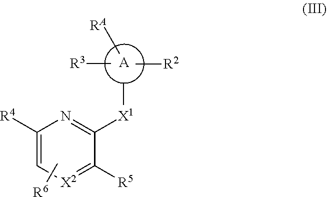
or a pharmaceutically acceptable salt, polymorph, prodrug, solvate or clathrate thereof, wherein:
A is heteroaryl;
R^{A} is H, alkyl, alkynyl, aryl, arylalkyl, OR⁷, C(O)OR⁷, cyano, amino, amido, or halo, wherein R⁷ is H, alkyl, arylalkyl or a protecting group;
R²—R⁶ are each independently H, alkyl, aryl, arylalkyl, OR⁷, cyano, amino, amido, or halo, wherein R⁷ is H, alkyl, arylalkyl or a protecting group;
X¹ is X³, —O—X⁴—, or —N(R⁸)—X⁴—, wherein X³ is alkyl (e.g., CH₂), X⁴ is a bond or alkyl, and R⁸ is H, alkyl, arylalkyl or acyl; and
X² is CR¹ or N;
or a pharmaceutically acceptable salt, polymorph, prodrug, solvate or clathrate thereof.

65. (canceled)

66. (canceled)

67. (canceled)

68. (canceled)

69. The method of claim 64, wherein A is 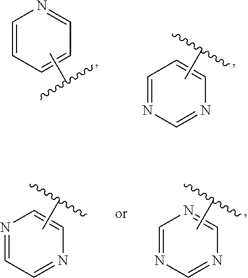
wherein each group can be substituted with R^{A}, R², and R³

70. The method of claim 64, wherein the compound is selected from the group consisting of: 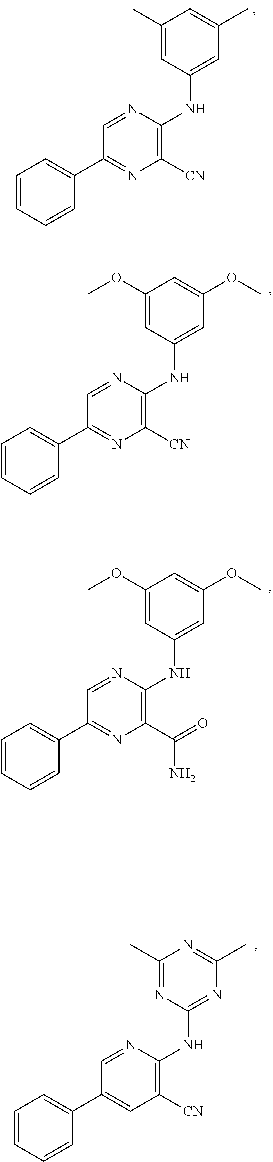 [SMILES: Cc1cc(C)cc(Nc2ncc(-c3ccccc3)nc2C#N)c1 ; COc1cc(Nc2ncc(-c3ccccc3)nc2C#N)cc(OC)c1 ; COc1cc(Nc2ncc(-c3ccccc3)nc2C(N)=O)cc(OC)c1 ; Cc1nc(C)nc(Nc2ncc(-c3ccccc3)cc2C#N)n1] 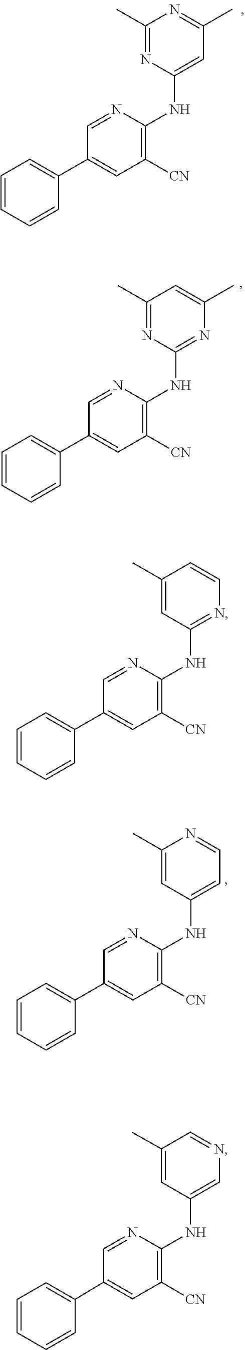 [SMILES: Cc1cc(Nc2ncc(-c3ccccc3)cc2C#N)nc(C)n1 ; Cc1cc(C)nc(Nc2ncc(-c3ccccc3)cc2C#N)n1 ; Cc1ccnc(Nc2ncc(-c3ccccc3)cc2C#N)c1 ; Cc1cc(Nc2ncc(-c3ccccc3)cc2C#N)ccn1 ; Cc1cncc(Nc2ncc(-c3ccccc3)cc2C#N)c1] 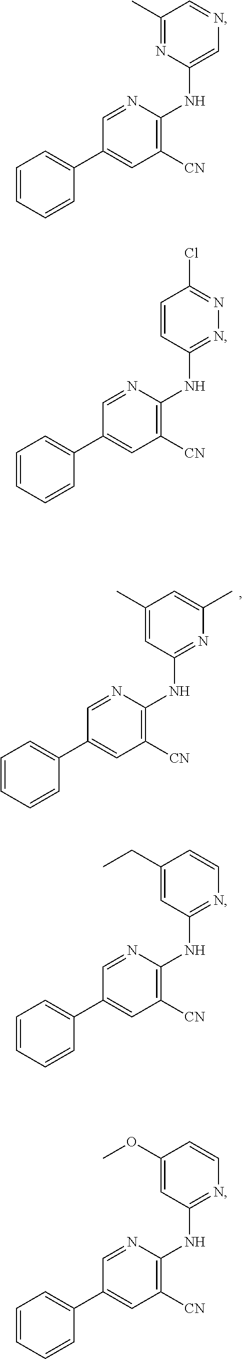 [SMILES: Cc1cncc(Nc2ncc(-c3ccccc3)cc2C#N)n1 ; N#Cc1cc(-c2ccccc2)cnc1Nc1ccc(Cl)nn1 ; Cc1cc(C)nc(Nc2ncc(-c3ccccc3)cc2C#N)c1 ; CCc1ccnc(Nc2ncc(-c3ccccc3)cc2C#N)c1 ; COc1ccnc(Nc2ncc(-c3ccccc3)cc2C#N)c1] 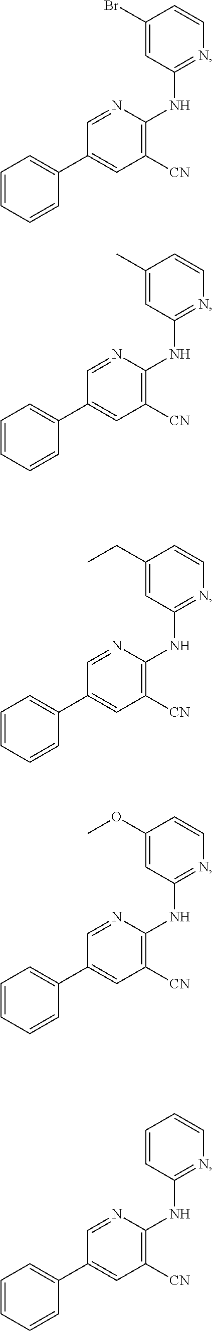 [SMILES: N#Cc1cc(-c2ccccc2)cnc1Nc1cc(Br)ccn1 ; Cc1ccnc(Nc2ncc(-c3ccccc3)cc2C#N)c1 ; CCc1ccnc(Nc2ncc(-c3ccccc3)cc2C#N)c1 ; COc1ccnc(Nc2ncc(-c3ccccc3)cc2C#N)c1 ; N#Cc1cc(-c2ccccc2)cnc1Nc1ccccn1] 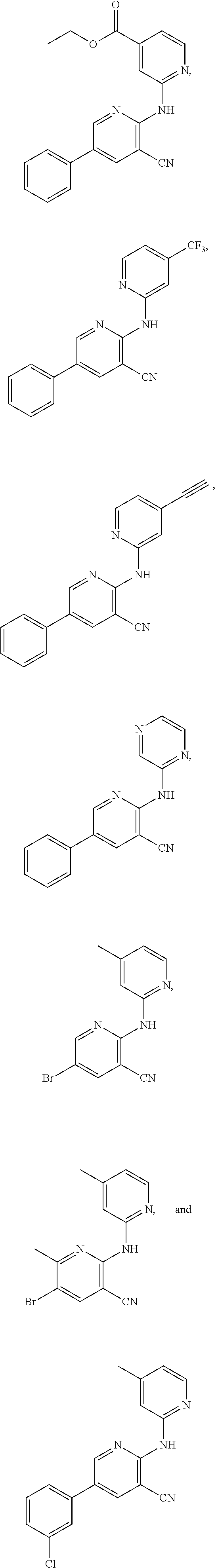
or a pharmaceutically acceptable salt, polymorph, prodrug, solvate or clathrate thereof.
